(19) United States
(12) Patent Application Publication
(10) Pub. No.: US 2020/0216432 A1
(43) Pub. Date: Jul. 9, 2020

(54) SULFONAMIDE DERIVATIVES AS STAT3 INHIBITORS FOR THE TREATMENT OF PROLIFERATIVE DISEASES

(71) Applicant: King's College London, London (GB)

(72) Inventors: David Edwin THURSTON, London (GB); Khondaker Mirazur RAHMAN, London (GB); Shirin JAMSHIDI, London (GB); Kazi Sharmin NAHAR, London (GB)

(73) Assignee: King's College London, London (GB)

(21) Appl. No.: 16/638,041

(22) PCT Filed: Aug. 10, 2018
(86) PCT No.: PCT/GB2018/052289

§ 371 Date: Feb. 10, 2020

(30) Foreign Application Priority Data
Aug. 11, 2017 (GB) 1712884.4

(51) Int. Cl. C07D 413/14; C07D 409/14; C07D 409/12; C07D 413/12; C07D 333/38; C07D 417/12; A61P 35/00

(52) U.S. Cl. CPC C07D 413/14; C07D 409/14; C07D 409/12; A61P 35/00; C07D 333/38; C07D 417/12; C07D 413/12

(57) ABSTRACT

The invention relates to novel derivatives of formula (I)

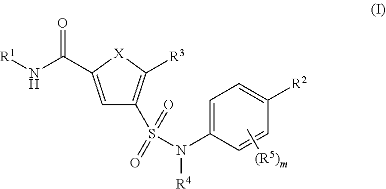

where R¹, R², R³, R⁴, R⁵, X and m are as defined in the specification. These compounds which have therapeutic activity, in particular, as STAT3 inhibitors and so are useful in the treatment of proliferative diseases or conditions such as cancer. Methods for producing these compounds, novel intermediates used in the methods, pharmaceutical compositions containing them and their use in therapy form further aspects of the invention.

### TECHNICAL FIELD

The present invention relates to novel compounds which have therapeutic activity, in particular, as STAT3 inhibitors for use in the treatment of proliferative diseases or conditions such as cancer, as well as to methods for producing these compounds, pharmaceutical compositions containing them and their use in therapy.

### BACKGROUND

Oncogenic transcription factors are an increasingly important target for anticancer therapies, as their inhibition could allow the “reprogramming” of tumour cells, leading to apoptosis or differentiation from the malignant phenotype.

STAT (signal transducer and activator of transcription) proteins—especially STAT3 and, to a large extent, also STATS—have emerged as promising molecular targets for the treatment of proliferative diseases and in particular cancer therapy. STAT3 induces the transcription of genes that control differentiation, inflammation, proliferation, and tumour cell invasion, and its over-expression has been implicated in many tumour types.

It is an attractive molecular target for novel cancer therapies, as a number of in vivo studies have shown that STAT3 is constitutively active in a variety of malignancies ranging from breast, prostate, and head and neck tumours to multiple myelomas and haematological cancer. Although cancer cells are often dependent upon activation of STAT3, non-cancerous cells are fairly tolerant of loss of its function, likely reflecting redundancies in normal signal transduction. Thus, STAT3 inhibitors have a high therapeutic potential.

Furthermore, resistance to targeted therapies often arises from activation of an alternative signalling pathway, many of which also converge on STATs. This suggests that inhibition of these proteins may forestall resistance. The STAT3 signalling pathway is stimulated by growth factors or cytokines which lead to receptor dimerization and activation. Phosphorylation of the tail of the receptor creates a docking site for the recruitment of un-phosphorylated STAT3 (uSTAT3) which becomes phosphorylated at the Tyr705 position (near the C-terminus) by JAK kinases. The phosphorylated STAT3 (pSTAT3) protein is then released, forming a homodimer through reciprocal binding of the SH2 domain of one monomer to the pTyr-containing PYLKTK sequence of another. This dimeric STAT3:STAT3 complex then translocates to the nucleus where it binds to its DNA consensus sequence, thus regulating transcription of numerous genes critical for the survival and proliferation of cancer cells.

There is a need for small molecule inhibitors of STAT3 and in particular STAT3:STAT:3 dimerization which have therapeutic activity. A range of pyrrolidine- or piperidine-sulphonyl-tolyl derivatives have been previously tested as IL-6 inhibitors (G. Zinzalla et al., Bioorg & Med Chem Lett., (2010), 20, 23, 7029-2031) and a compound of formula (A)

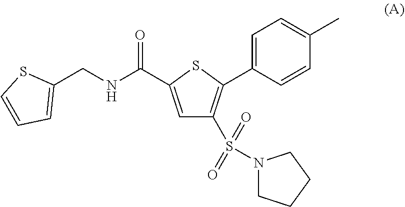 [SMILES: Cc1ccc(-c2sc(C(=O)NCc3cccs3)cc2S(=O)(=O)N2CCCC2)cc1]

herein designated RH-06, was identified as being a novel small molecule inhibitor. The authors reported that following IL-6 stimulation, the compound selectively inhibited phosphorylation of STAT3 and that it was selectively cytostatic in STAT3 dependent cells as compared to STAT3-null cells. The applicants have found that RH-06 in fact inhibits STAT3 dimerization (unpublished results).

The applicants have designed and produced of a novel series of potent STAT3 inhibitors.

### SUMMARY OF THE INVENTION

According to the present invention there is provided a compound of formula (I)

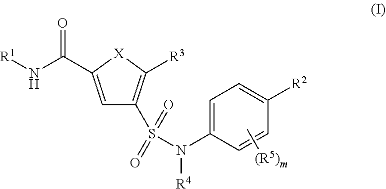

where X is oxygen, sulfur, NR¹¹ or CH₂, where R¹¹ is H or alkyl;

R¹ is an aryl, aralkyl group, heteroaryl group or heteroarylalkyl group; all of which are substituted by one or more groups selected from alkoxycarbonyl, aryl, aralkyl, arylalkoxy, heterocyclyl, heterocyloalkyl or heterocycloalkoxy group, any of which may be optionally substituted;

R² is a group of formula COR^{E} where R⁶ is hydrogen or a group OR⁷ where R⁷ is hydrogen, alkyl, alkenyl, alkynyl, cycloalkyl, aryl, heterocyclyl, amino, alkylamino or dialkylamino;

R³ is hydrogen, halo, nitro, cyano, carboxy, optionally substituted alkyl, optionally substituted alkenyl, optionally substituted alkynyl, optionally substituted aryl, optionally substituted cycloalkyl, or optionally substituted heterocyclic group;

R⁴ is hydrogen, C₁₋₄ alkyl or CF₃ group;

R⁵ is a substituent and each R⁵ is independently selected from hydroxy, C₁₋₄alkyl (such as methyl or ethyl), C₁₋₄alkoxy (such as methoxy), halo, amino, C₁₋₄alkylamino, C₁₋₄dialkylamino, nitro, cyano, thiol, trifluoromethyl

m is 0, 1, 2 or 3;

or a tautomer or a pharmaceutically acceptable salt thereof.

Compounds of formula (I) represent a novel series of small molecule inhibitors of STAT3:STAT3 dimerization which act as potent and selective agents capable of down regulating this signalling pathway. These compounds have therapeutic application in the treatment of conditions in which STAT3 is implicated, in particular in the treatment of proliferative diseases such as cancer.

Without being bound by theory, it is believed that compounds of formula (I) disrupt the

STAT3 dimerisation by interacting with the hexapeptide pocket of the STAT3 SH2 domain. This has been confirmed by in silico modelling studies.

As used herein, the expression “alkyl” refers to saturated chains of carbon atoms, which may be straight or branched which, unless otherwise stated, suitably contain from 1-10 carbon atoms, for instance from 1-6 carbon atoms and in particular from 1-4 carbon atoms. The expressions “alkenyl” and “alkynyl” refers to unsaturated chains of carbon atoms, which may be straight or branched which, unless otherwise stated, suitably contain from 2-10 carbon atoms, for instance from 2-6 carbon atoms and in particular from 2-4 carbon atoms. The expression ‘alkoxy’ refers to -0-alkyl groups, where alkyl is as defined above. The expression ‘cycloalkyl’ refers to cyclic alkyl groups, forming one or more ring structures.

The expression ‘heterocyclic group’ refers to a saturated or unsaturated ring structures containing from 3-20 atoms, at least one of which is a heteroatom selected from oxygen, sulfur or nitrogen. Rings may be aromatic in nature, or, in the case of fused rings, they may comprise both aromatic and non-aromatic rings. They may be monocyclic rings or they comprise fused bi- or tri-cyclic ring systems. Particular examples of heterocylic groups include mono-cyclic rings comprising from 4-7 ring atoms, in particular from 5-6 ring atoms. Suitably the heterocyclic rings comprise 1 or 2 heteroatoms, which may be selected in particular from nitrogen or oxygen. Examples of such groups which are saturated include pyrrolidine, tetrahydrofuran, imidazolidinyl, pyrazolidinyl, oxazolidinyl, isoxazolidinyl, thiazolidinyl, isothiazolidinyl, piperazinyl, tetrahydropyranyl, morpholinyl, thiomorpholinyl or dioxanyl groups. Unsaturated and in particular aromatic heterocyclic groups are exemplified below in the definition of ‘heteroaryl’.

The expression ‘heteroaryl’ refers specifically the aromatic heterocyclic groups of from 5 to 20 atoms. Particular examples are 5 or 6-membered aromatic rings containing at least one heteroatom as described above, such as pyrrolyl, furyl, thiophenyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyridyl, pyranyl, thiopyanyl, diazinyl, oxazinyl, thiazinyl, dioxinyl, triazinyl or tetrazinyl.

The expression ‘aryl’ group refers to aromatic carbocyclic groups. They may comprise one or more fused rings, such as phenyl or naphthyl.

The expression ‘aralkyl’ refers to alkyl groups which carry an aryl substituent such as benzyl.

The term ‘halo’ refers to fluoro, chloro, bromo or iodo groups.

As used herein the expression ‘pharmaceutically acceptable salt’ refers to non-toxic, physiologically acceptable salts. Particular examples include alkali metal salts such as sodium, lithium or potassium salts; alkaline earth metal salts such as aluminum, calcium or magnesium salts; or ammonium salts.

In a particular embodiment, R¹ is an aryl, aralkyl group, heteroaryl group or heteroarylalkyl group, which is substituted by an aryl, aralkyl, arylalkoxy, heterocyclyl, heterocyloalkyl or heterocycloalkoxy group, any of which may be optionally substituted. Suitable optional substituents for the aryl, aralkyl, arylalkoxy, heterocyclyl, heterocyloalkyl or heterocycloalkoxy groups are one or more alkyl groups, such as C₁₋₄ alkyl groups including methyl.

In a particular embodiment, the group X is S or O, and in particular is S.

When X is a group NR¹¹, R¹¹ is suitably hydrogen or C₁₋₄ alkyl such as methyl or ethyl.

In a particular embodiment R¹ is a group of sub-formula (i)

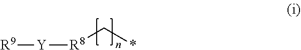

where * is the point of attachment,

n is 0 or an integer of from 1 to 6,

R⁸ is an aryl or heteroaryl group,

Y is a bond, a carbonyl group or an alkylene spacer group of from 1 to 6 atoms, optionally interposed with a heteroatom such as oxygen, nitrogen or sulfur or a carbonyl group; and

R⁹ is an aryl or heterocyclic group, either of which may be optionally substituted by an alkyl group.

In a particular embodiment, n is 0, 1 or 2, such as 0 or 1.

In a particular embodiment, R⁸ is an aryl group such as a phenyl group.

Suitably Y is a bond or a C₁₋₄alkylene group, such as a methylene or ethylene group, or an alkylenoxy group such as ethylenoxy.

In a particular embodiment, R⁹ is a non-aromatic heterocyclic group, in particular, a morpholinyl, piperidyl, piperazinyl or N-methyl piperazinyl group.

In a particular embodiment, the group of sub-formula (i) is a group of sub-formula (ia)

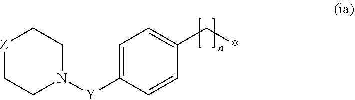

where n and Y are as defined above, and Z is a CH₂, O or NR¹⁰ group where R¹⁹ is hydrogen or methyl.

A particular example of a group of sub-formula (ia) is 2-morpholinoethoxyphenyl. Another particular example is piperidin-1-ylmethyl-benzyl.

In a particular embodiment R² is a carboxylic ester group. In particular, R² is a group COOR⁷ where R⁷ is a C₁₋₃ alkyl group, in particular methyl.

In a particular embodiment m is 0 or 1, and in particular is 0.

Suitable optional substituents for optionally substituted alkyl, optionally substituted alkenyl, optionally substituted alkynyl, optionally substituted aryl, optionally substituted cycloalkyl, or optionally substituted heterocyclic groups, R³, include nitrile or heterocyclic groups which are optionally substituted by one or more alkyl groups.

In a particular embodiment R³ is a group of sub-formula (ii)

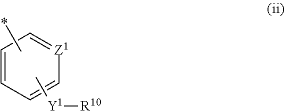

where * is the point of attachment, Z¹ is —CH═ or —N═, Y¹ is a bond, a carbonyl group or an alkylene chain of from 1 to 4 carbon atoms, optionally interposed with a heteroatom such as oxygen, nitrogen or sulfur or a carbonyl group, and R¹⁰ is an optionally substituted heterocyclic group, in particular a non-aromatic heterocyclic group such as morpholinyl, piperidinyl, piperazinyl or N-alkylpiperazinyl, such as n-methylpiperazinyl.

In a particular embodiment, the substituent Y¹-R¹⁰ is located at the para-position on the aryl or heteroaryl ring.

In another embodiment, the substituent Y¹-R¹⁰ is located at the meta-position on the aryl or heteroaryl ring.

In a particular embodiment Z¹ is —CH═.

In another particular embodiment Z¹ is —N═.

Particular examples of groups of sub-formula (ii) are compounds of sub-formula (iia)

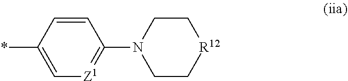

where Z¹ is as defined above and in particular is —CH═ or N, and R¹² is —CH₂, NH or N-methyl, and * is the point of attachment.

Particular examples of groups of sub-formula (ii) or (iia) include 4-(piperazin-1-yl)pyridine-3-yl or 4-(4-methyl piperazin-1-yl)pyridine-3-yl.

Another particular example is (piperidin-1-yl)phenyl.In another embodiment, R³ is a halo group such as bromo.

In a particular embodiment, R⁴ is hydrogen or methyl, and in particular is methyl.

Examples of compounds of formula (I) are shown in the following Table 1

TABLE 1

| Compound | |
|---|---|
| No | Compound structure |
| 1 | 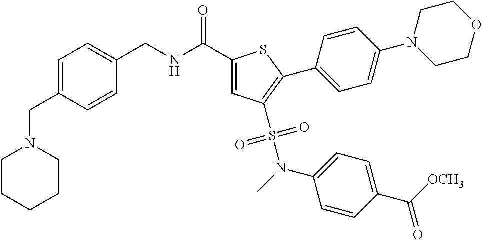 [SMILES: COC(=O)c1ccc(N(C)S(=O)(=O)c2cc(C(=O)NCc3ccc(CN4CCCCC4)cc3)sc2-c2ccc(N3CCOCC3)cc2)cc1] |
| 2 | 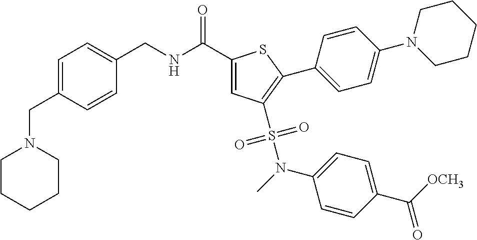 [SMILES: COC(=O)c1ccc(N(C)S(=O)(=O)c2cc(C(=O)NCc3ccc(CN4CCCCC4)cc3)sc2-c2ccc(N3CCCCC3)cc2)cc1] |
| 3 | 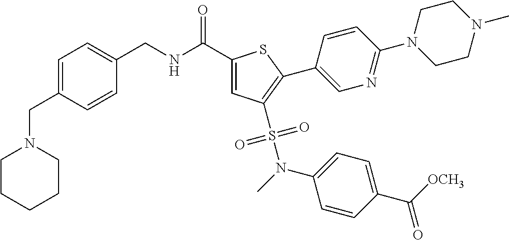 [SMILES: COC(=O)c1ccc(N(C)S(=O)(=O)c2cc(C(=O)NCc3ccc(CN4CCCCC4)cc3)sc2-c2ccc(N3CCN(C)CC3)nc2)cc1] |
| 4 | 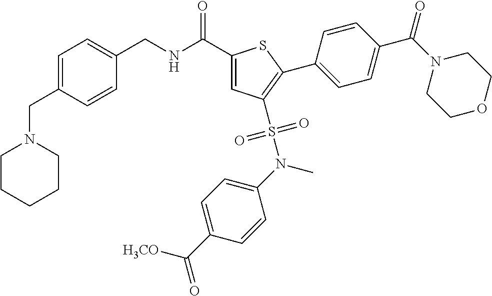 [SMILES: COC(=O)c1ccc(N(C)S(=O)(=O)c2cc(C(=O)NCc3ccc(CN4CCCCC4)cc3)sc2-c2ccc(C(=O)N3CCOCC3)cc2)cc1] |
| 5 | 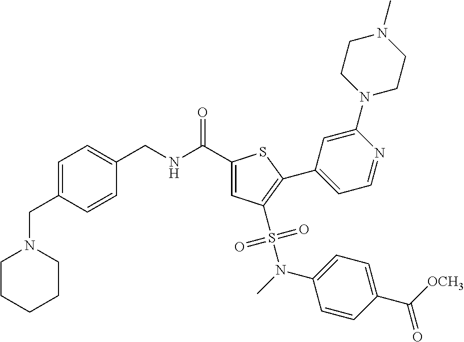 [SMILES: COC(=O)c1ccc(N(C)S(=O)(=O)c2cc(C(=O)NCc3ccc(CN4CCCCC4)cc3)sc2-c2ccnc(N3CCN(C)CC3)c2)cc1] |
| 6 | 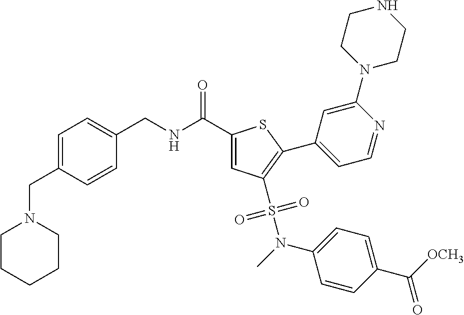 [SMILES: COC(=O)c1ccc(N(C)S(=O)(=O)c2cc(C(=O)NCc3ccc(CN4CCCCC4)cc3)sc2-c2ccnc(N3CCNCC3)c2)cc1] |
| 7 | 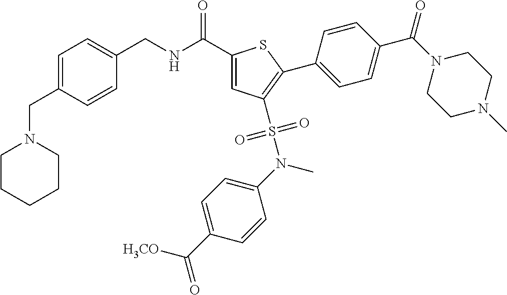 [SMILES: COC(=O)c1ccc(N(C)S(=O)(=O)c2cc(C(=O)NCc3ccc(CN4CCCCC4)cc3)sc2-c2ccc(C(=O)N3CCN(C)CC3)cc2)cc1] |
| 8 | 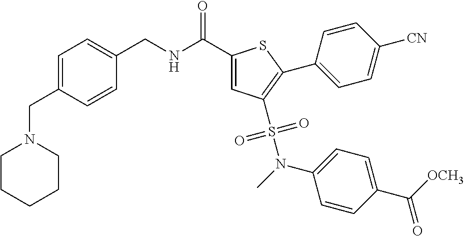 [SMILES: COC(=O)c1ccc(N(C)S(=O)(=O)c2cc(C(=O)NCc3ccc(CN4CCCCC4)cc3)sc2-c2ccc(C#N)cc2)cc1] |
| 9 | 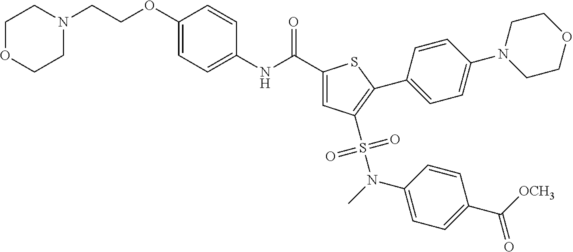 [SMILES: COC(=O)c1ccc(N(C)S(=O)(=O)c2cc(C(=O)Nc3ccc(OCCN4CCOCC4)cc3)sc2-c2ccc(N3CCOCC3)cc2)cc1] |
| 10 | 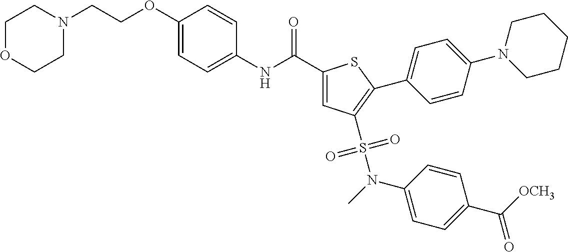 [SMILES: COC(=O)c1ccc(N(C)S(=O)(=O)c2cc(C(=O)Nc3ccc(OCCN4CCOCC4)cc3)sc2-c2ccc(N3CCCCC3)cc2)cc1] |
| 11 | 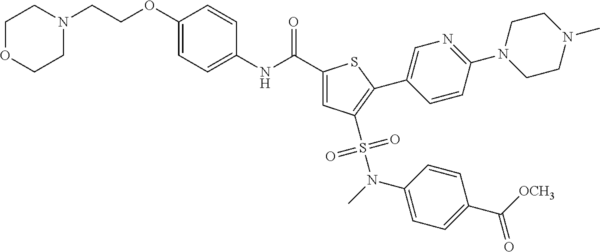 [SMILES: COC(=O)c1ccc(N(C)S(=O)(=O)c2cc(C(=O)Nc3ccc(OCCN4CCOCC4)cc3)sc2-c2ccc(N3CCN(C)CC3)nc2)cc1] |
| 12 | 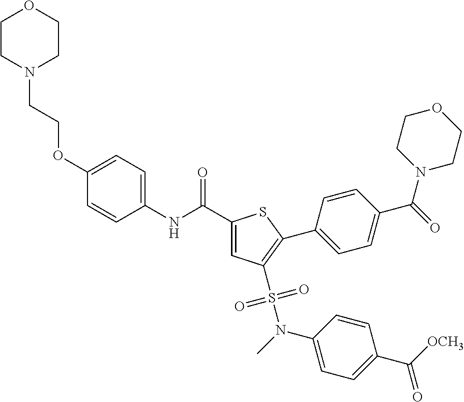 [SMILES: COC(=O)c1ccc(N(C)S(=O)(=O)c2cc(C(=O)Nc3ccc(OCCN4CCOCC4)cc3)sc2-c2ccc(C(=O)N3CCOCC3)cc2)cc1] |
| 13 | 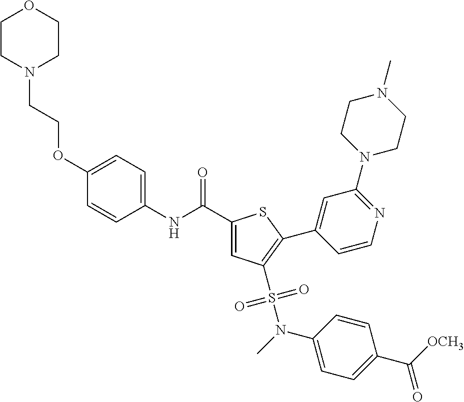 [SMILES: COC(=O)c1ccc(N(C)S(=O)(=O)c2cc(C(=O)Nc3ccc(OCCN4CCOCC4)cc3)sc2-c2ccnc(N3CCN(C)CC3)c2)cc1] |
| 14 | 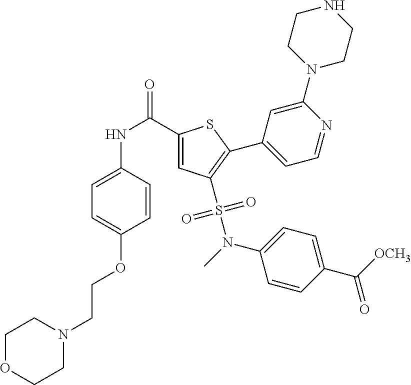 [SMILES: COC(=O)c1ccc(N(C)S(=O)(=O)c2cc(C(=O)Nc3ccc(OCCN4CCOCC4)cc3)sc2-c2ccnc(N3CCNCC3)c2)cc1] |
| 15 | 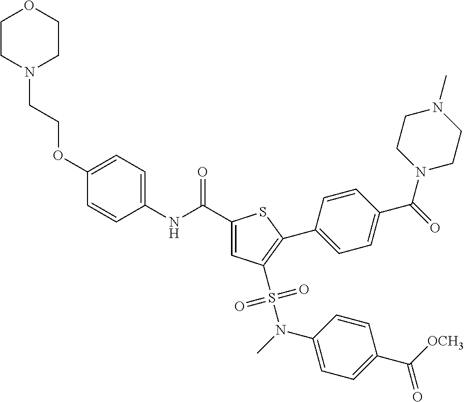 [SMILES: COC(=O)c1ccc(N(C)S(=O)(=O)c2cc(C(=O)Nc3ccc(OCCN4CCOCC4)cc3)sc2-c2ccc(C(=O)N3CCN(C)CC3)cc2)cc1] |
| 16 | 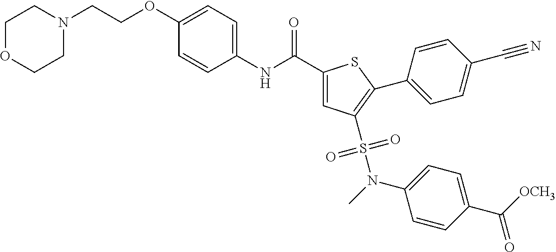 [SMILES: COC(=O)c1ccc(N(C)S(=O)(=O)c2cc(C(=O)Nc3ccc(OCCN4CCOCC4)cc3)sc2-c2ccc(C#N)cc2)cc1] |
| 17 | 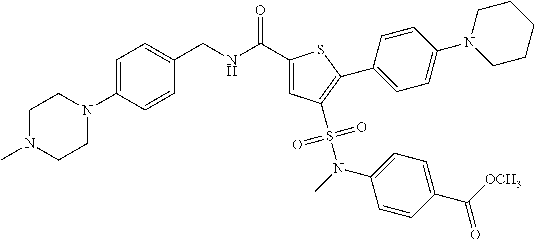 [SMILES: COC(=O)c1ccc(N(C)S(=O)(=O)c2cc(C(=O)NCc3ccc(N4CCN(C)CC4)cc3)sc2-c2ccc(N3CCCCC3)cc2)cc1] |
| 18 | 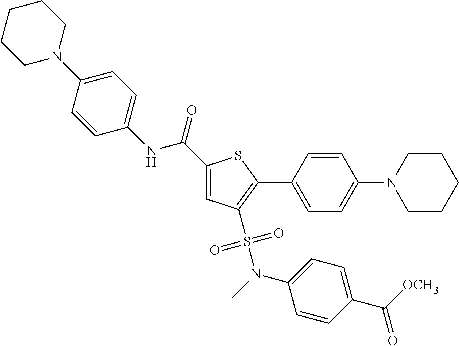 [SMILES: COC(=O)c1ccc(N(C)S(=O)(=O)c2cc(C(=O)Nc3ccc(N4CCCCC4)cc3)sc2-c2ccc(N3CCCCC3)cc2)cc1] |
| 19 | 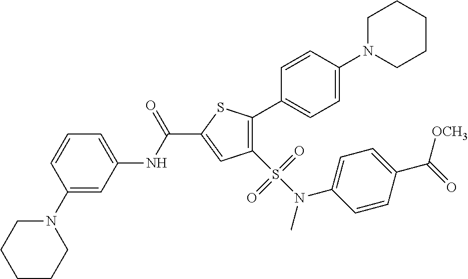 [SMILES: COC(=O)c1ccc(N(C)S(=O)(=O)c2cc(C(=O)Nc3cccc(N4CCCCC4)c3)sc2-c2ccc(N3CCCCC3)cc2)cc1] |
| 20 | 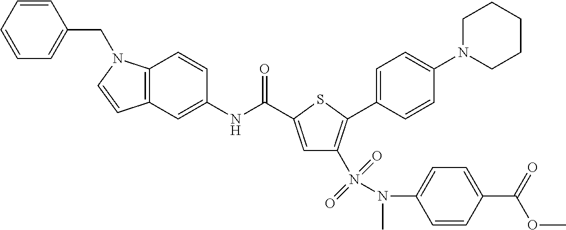 |
| 21 | 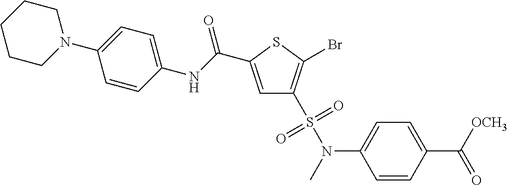 [SMILES: COC(=O)c1ccc(N(C)S(=O)(=O)c2cc(C(=O)Nc3ccc(N4CCCCC4)cc3)sc2Br)cc1] |
| 22 | 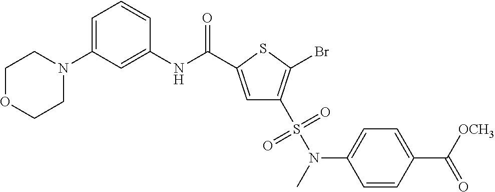 [SMILES: COC(=O)c1ccc(N(C)S(=O)(=O)c2cc(C(=O)Nc3cccc(N4CCOCC4)c3)sc2Br)cc1] |
| 23 | 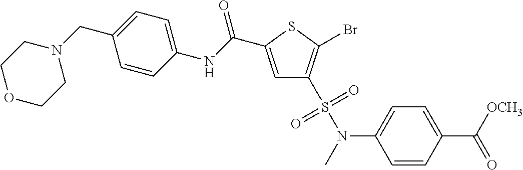 [SMILES: COC(=O)c1ccc(N(C)S(=O)(=O)c2cc(C(=O)Nc3ccc(CN4CCOCC4)cc3)sc2Br)cc1] |
| 24 | 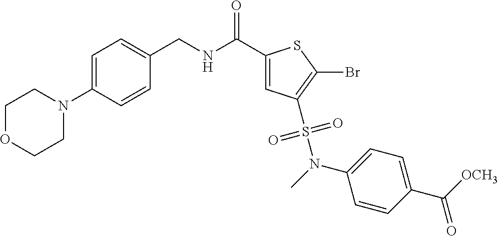 [SMILES: COC(=O)c1ccc(N(C)S(=O)(=O)c2cc(C(=O)NCc3ccc(N4CCOCC4)cc3)sc2Br)cc1] |
| 25 | 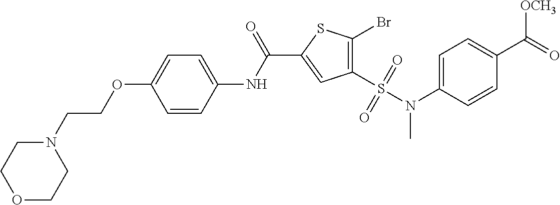 [SMILES: COC(=O)c1ccc(N(C)S(=O)(=O)c2cc(C(=O)Nc3ccc(OCCN4CCOCC4)cc3)sc2Br)cc1] |
| 26 | 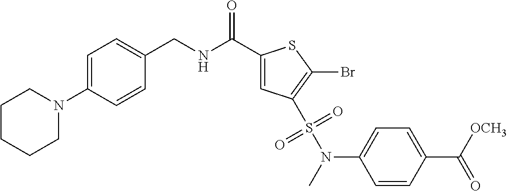 [SMILES: COC(=O)c1ccc(N(C)S(=O)(=O)c2cc(C(=O)NCc3ccc(N4CCCCC4)cc3)sc2Br)cc1] |
| 27 | 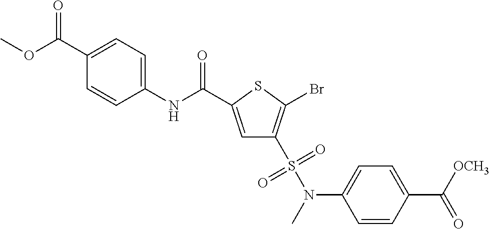 [SMILES: COC(=O)c1ccc(NC(=O)c2cc(S(=O)(=O)N(C)c3ccc(C(=O)OC)cc3)c(Br)s2)cc1] |
| 28 | 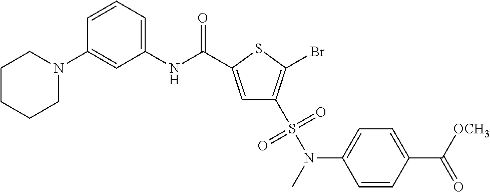 [SMILES: COC(=O)c1ccc(N(C)S(=O)(=O)c2cc(C(=O)Nc3cccc(N4CCCCC4)c3)sc2Br)cc1] |
| 29 | 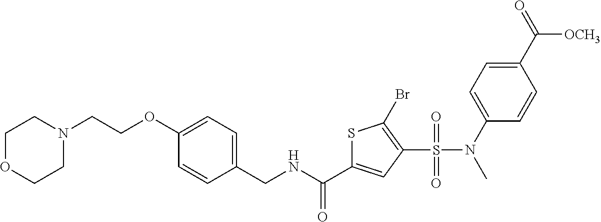 [SMILES: COC(=O)c1ccc(N(C)S(=O)(=O)c2cc(C(=O)NCc3ccc(OCCN4CCOCC4)cc3)sc2Br)cc1] |
| 30 | 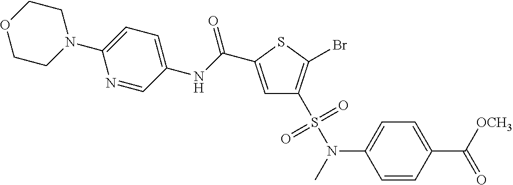 [SMILES: COC(=O)c1ccc(N(C)S(=O)(=O)c2cc(C(=O)Nc3ccc(N4CCOCC4)nc3)sc2Br)cc1] |
| 31 | 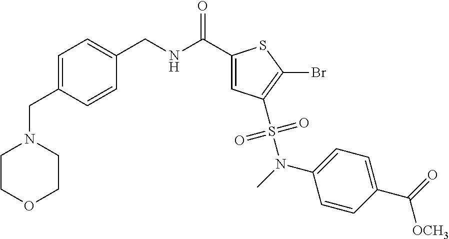 [SMILES: COC(=O)c1ccc(N(C)S(=O)(=O)c2cc(C(=O)NCc3ccc(CN4CCOCC4)cc3)sc2Br)cc1] |
| 32 | 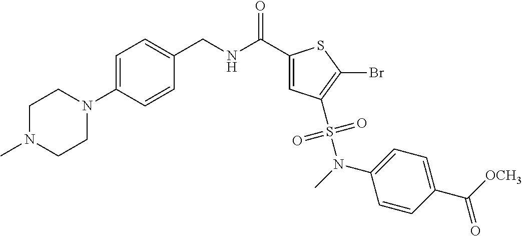 [SMILES: COC(=O)c1ccc(N(C)S(=O)(=O)c2cc(C(=O)NCc3ccc(N4CCN(C)CC4)cc3)sc2Br)cc1] |
| 33 | 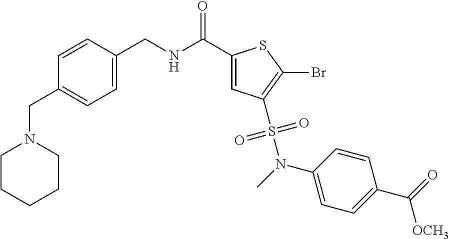 [SMILES: COC(=O)c1ccc(N(C)S(=O)(=O)c2cc(C(=O)NCc3ccc(CN4CCCCC4)cc3)sc2Br)cc1] |
| 34 | 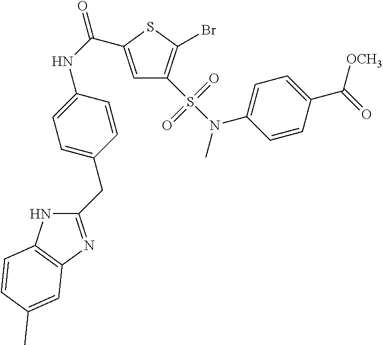 [SMILES: COC(=O)c1ccc(N(C)S(=O)(=O)c2cc(C(=O)Nc3ccc(Cc4nc5cc(C)ccc5[nH]4)cc3)sc2Br)cc1] |
| 35 | 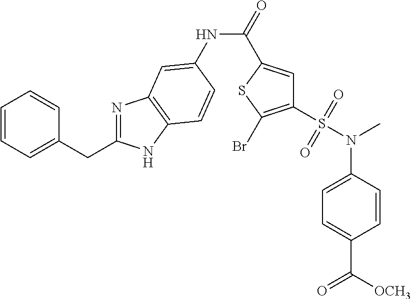 [SMILES: COC(=O)c1ccc(N(C)S(=O)(=O)c2cc(C(=O)Nc3ccc4[nH]c(Cc5ccccc5)nc4c3)sc2Br)cc1] |
| 36 | 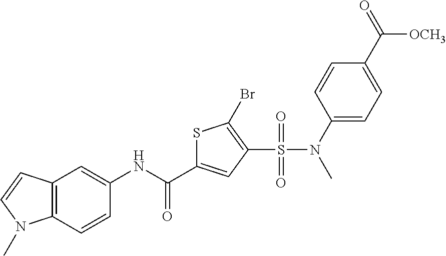 [SMILES: COC(=O)c1ccc(N(C)S(=O)(=O)c2cc(C(=O)Nc3ccc4c(ccn4C)c3)sc2Br)cc1] |
| 37 | 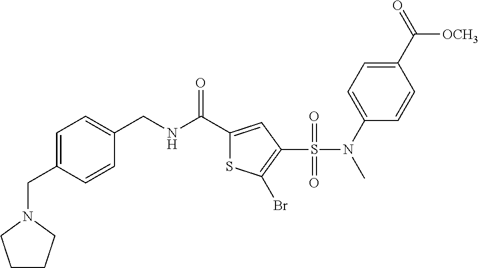 [SMILES: COC(=O)c1ccc(N(C)S(=O)(=O)c2cc(C(=O)NCc3ccc(CN4CCCC4)cc3)sc2Br)cc1] |
| 38 | 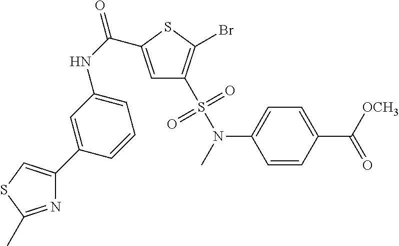 [SMILES: COC(=O)c1ccc(N(C)S(=O)(=O)c2cc(C(=O)Nc3cccc(-c4csc(C)n4)c3)sc2Br)cc1] |
| 39 | 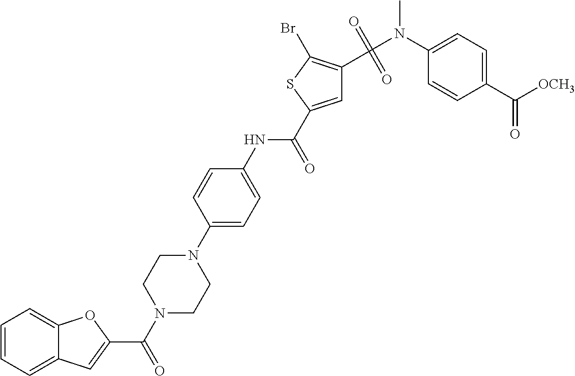 |
| 40 | 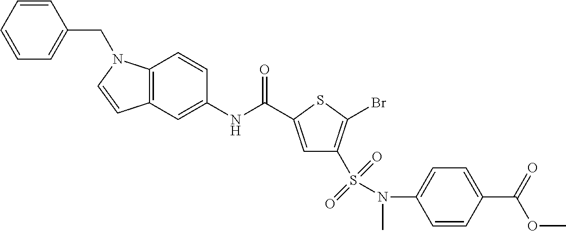 [SMILES: COC(=O)c1ccc(N(C)S(=O)(=O)c2cc(C(=O)Nc3ccc4c(ccn4Cc4ccccc4)c3)sc2Br)cc1] |

Compounds of formula (I) may be prepared using conventional methods as would be understood in the art. In a particular embodiment of the invention, compounds of formula (I) are suitably prepared by reacting a compound of formula (II)

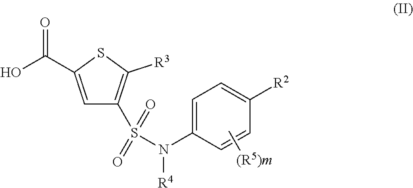

where X, R², R³, R⁴, R⁵ and m are as defined above, with a compound of formula (III)

where R¹ is as defined above. The reaction is suitably carried out in an organic solvent such as dimethylformamide (DMF) at temperatures in the range of from 0 to 50° C., and conveniently at ambient temperature. The reaction may be carried out in the presence of a coupling agent such as hydroxybenzotriazole (HOBt) and diisopropylcarbodiimide (DIC) or dicyclohexylcarbodiimide (DCC). The reaction may be allowed to proceed for a prolonged period of for example from 12-24 hours, to achieve a reasonable yield of the compound of formula (I) or a salt thereof. If required, a group R³ may be changed for a different such group, for example as illustrated below.

Compounds of formula (II) where R³ is other than hydrogen, halo or nitro may be prepared using a C—C bond forming reaction as would be understood in the art. In a particular embodiment, the compound of formula (II) is formed by reacting a compound of formula (IV)

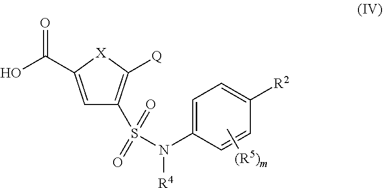

where R², R⁴, R⁵ and m are as defined above, and Q is a leaving group, in particular a Suzuki leaving group such as halo (in particular bromo) or triflate; with a boronic acid of formula (V)

R³—B(OH)₂ (V)

where R³ is as defined above. The conditions under which the reaction is carried out are suitably those conventionally used in a Suzuki coupling reaction. For instance, the reaction is carried out in an organic solvent such as toluene or benzene, in the presence of an excess of a carbonate such as potassium carbonate and a palladium catalyst such as Pd(PPh₃). The reaction may be carried out at temperatures in the range of from 20-100° C. and conveniently under microwave conditions.

Compounds of formula (IV) are suitably prepared by reacting a compound of formula (VI)

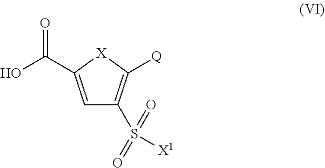

where X and Q are as defined above, and X¹ is a leaving group, such as a halo group, such as chloro, fluoro or mesylate or tosylate, with a compound of formula (VII)

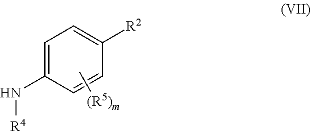

where R², R⁴, R⁵ and m are as defined above. The reaction is suitably effected in an organic solvent such as an alkyl alcohol, for instance methanol, in the presence of a nucleophilic catalyst such as 4-dimethylaminopyridine (DMAP). Temperatures of from 0 to 50° C. and conveniently ambient temperature are employed.

Compounds of formula (VI) may be prepared by reacting a compound of formula (VIII)

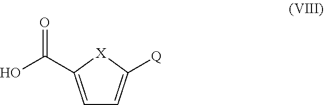

where X and Q are as defined above, with a compound of formula (IX)

X¹SO₃H (IX)

where X¹ is as defined above. The reaction is carried out in an organic solvent such as an alkyl alcohol, for example methanol, at elevated temperatures, and conveniently at the reflux temperature of the solvent.

In an alternative method for preparing compounds of formula (I), a compound of formula (XI)

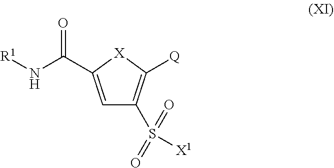

where R¹, R³, X and X¹ are as defined above, is reacted with a compound of formula (VII) as defined above. Suitable reaction conditions will be similar to those used to produce compounds of formula (IV) from the compounds of formula (VI) and (VII). Again, a group R³ may, if required, be changed to a different such group.

Alternatively, to produce compounds of formula (I) where R³ is other than hydrogen, halo or nitro, compounds of formula (X)

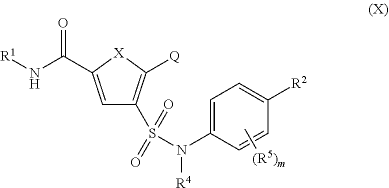

where X, Q, R¹, R², R⁴, R⁵ and m are as defined above, are reacted with a compound of formula (V) as defined above. Suitable reaction conditions will be similar to those used to produce compounds of formula (II) from the compounds of formula (IV) and (V).

Compounds of formula (X) where Q is halo, such as bromo, are compounds of formula (I), and so may not require modification. However, in a particular embodiment, compounds of formula (I) where R³ is halo is converted to a compound of formula (I) where R³ is a different such group, such as a group of sub-formula (ii) above. In particular, conversion of compounds of formula (I) where R³ is halo is converted to a compound of formula (I) where R³ is a different such group may be achieved for example by Suzuki coupling reaction with a boronic acid of formula (V), as discussed above. Compounds of formula (X) are suitably prepared by reacting compounds of formula (IV) as defined above with a compound of formula (III) as defined above. Suitable reaction conditions will be similar to those described above for the reaction of the compound of formula (II) with the compound of formula (III). Alternatively, they may be prepared by reacting a compound of formula (XII)

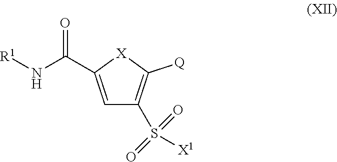

where R¹, Q, X and X¹ are as defined above, are reacted with a compound of formula (VII) as defined above.

Compounds of formula (II), (IV) and (X) are novel compounds and therefore form a further aspect of the invention. They may have STAT3 inhibitory activity in their own right, in particular, the compounds of formula (IV) and (X) and so pharmaceutical compositions containing them and their use in therapy, including methods of treating proliferative disease or condition in humans or animals using said compounds form a further aspect of the invention.

Compounds of formula (III), (V), (VI), (VII), (VIII), (XI) or (XII) are either known compounds or they can be prepared from known compounds by conventional methods.

Compounds of the invention may be used to inhibit STAT3 and thus are useful in therapy, for example in the treatment of proliferative diseases or condition such as cancer.

For use in therapy, the compounds are suitably in the form of a pharmaceutical composition.

Thus in a further aspect, there is provided a pharmaceutical composition comprising a compound of formula (I) in combination with a pharmaceutically acceptable carrier.

As used herein, the expression ‘pharmaceutical composition’ or ‘pharmaceutically acceptable carrier’ encompasses veterinary compositions and veterinarily acceptable carriers respectively, when the compositions are used to treat non-human animals as explained further below.

Suitable pharmaceutical compositions will be in either solid or liquid form. They may be adapted for administration by any convenient route, such as parenteral, oral or topical administration or for administration by inhalation or insufflation. The pharmaceutical acceptable carrier may include diluents or excipients which are physiologically tolerable and compatible with the active ingredient.

Parenteral compositions are prepared for injection, for example either subcutaneously or intravenously. They may be liquid solutions or suspensions, or they may be in the form of a solid that is suitable for solution in, or suspension in, liquid prior to injection. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH-buffering agents, and the like.

Oral formulations will be in the form of solids or liquids, and may be solutions, syrups, suspensions, tablets, pills, capsules, sustained-release formulations, or powders. Oral formulations include such normally employed excipients as, for example, pharmaceutical grades of mannitol, lactose, starch, magnesium stearate, sodium saccharin, cellulose, magnesium carbonate, and the like.

Topical formulations will generally take the form of suppositories or intranasal aerosols. For suppositories, traditional binders and excipients may include, for example, polyalkylene glycols or triglycerides; such suppositories may be formed from mixtures containing the compound of formula (I).

In yet a further aspect, the invention provides a method of treating a disease or condition by inhibiting SAT3, said method comprising administering to a patient in need thereof, an effective amount of a compound of formula (I), or a pharmaceutical composition comprising a compound of formula (I).

The amount of compound of formula (I) administered will vary in accordance with normal clinical practice and will depending upon factors such as the nature of the reagent being used, the size and health of the patient, the nature of the condition being treated etc. in accordance with normal clinical practice. Typically, a dosage in the range of from 1 μg-50 mg/Kg for instance from 2-20 mg/Kg, such as from 5-15 mg/Kg of the compound of formula (I) would be expected to produce a suitable effect.

Patients may be any animal, including human and non-human animals including for example, mice, rats, rabbits, dogs, cats, pigs, horses, camels, sheep, goats, cattle and non-human primates, including, but not limited to, monkeys and chimpanzees. In a particular embodiment, the patients treated are humans.

In particular, the disease in which inhibition of SAT3 may be beneficial and which is therefore susceptible to treatment using the method of the invention is proliferative disease.

Examples of proliferative conditions include, but are not limited to, benign, pre-malignant, and malignant cellular proliferation, including but not limited to, neoplasms and tumours (e.g. histocytoma, glioma, astrocyoma, osteoma), cancers (e.g. breast cancer, pancreatic cancer, prostate cancer, head and neck tumours, cervical cancer, colon cancer, lung cancer, stomach cancer, kidney cancer, bladder cancer, bowel cancer, small cell lung cancer, gastrointestinal cancer, ovarian carcinoma, testicular cancer, liver cancer, brain cancer, sarcoma, osteosarcoma, Kaposi's sarcoma, melanoma, multiple myelomas and haematological cancers such as leukemias), psoriasis, bone diseases, fibroproliferative disorders (e.g. of connective tissues), and atherosclerosis.

In a particular embodiment, the proliferative disease is cancer. Examples of such cancers may include breast cancer, pancreatic cancer, prostate cancer, head and neck tumours, cervical cancer, colon cancer, lung cancer, stomach cancer, kidney cancer, bladder cancer, bowel cancer, multiple myelomas and haematological cancers such as leukemia.

In yet a further aspect, the invention provides a compound of formula (I) for use in the treatment of proliferative disease.

In a particular embodiment, the proliferative disease is breast cancer, such as triple negative cancer.

In another embodiment, the proliferative disease is leukemia.

The applicants have developed a range of compounds, based upon the central hypothesis that a selective and more potent STAT3 SH2 domain targeting inhibitor would be achieved by combining multiple small-pharmacophores that had been previously shown to interact with the SH2 domain. A range of compounds belonging to different structural scaffolds and designed using in silico methods, were successfully synthesised, characterised using spectroscopic techniques and subsequently evaluated in both cell-free and cellular assays. Compounds of the invention showed significantly high STAT3-dimerisation inhibition and significant cytotoxicity in the MDA MB 231 STAT3-dependent cell line.

Certain compounds of the invention were evaluated by RT-PCR to understand the effect of STAT3-dimerisation inhibition on the down-regulation of STAT3-dependent genes. These compounds produced notable down-regulation of STAT3 itself and also the STAT3-dependent genes Bcl-2, cyclin D1 and fascin, while not down-regulating STAT1. These results suggest a potential relationship between the decreased viability of STAT3-dependent MDA-MB-231 cells in the presence of the agents, and the down-regulation of STAT3-dependent genes. No down-regulation of the housekeeping gene GAPDH was observed.

A compound of the invention was also evaluated in a preliminary in vivo efficacy assay in immune-compromised mice bearing MDA MB 231 tumours, and was found to have significant tumour growth inhibition properties.

Throughout the description and claims of this specification, the words “comprise” and “contain” and variations of the words, for example “comprising” and “comprises”, mean “including but not limited to”, and do not exclude other components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 shows a series of RT-PCR gels showing the effect of compounds of the invention on the expression of STAT3 and STAT3-dependent genes in a STAT3-dependent MDA-MB-231 cell line (lane 1, untreated cells; lane 2,500 μg/ml LPS for 8 h; lane 3, 500 μg/ml LPS for 8 h, 25 μM 1 for 8 h);

FIG. 2 shows the effect of a compound of the invention on the weight of a mouse as a marker of a general toxicity; and

FIG. 3 shows the effect of treatment of a tumour bearing mouse with ligand 6.6 compared to untreated control mice. Dosing was on days 1, 3 and 5 as indicated by the dashed vertical lines.

### DETAILED DESCRIPTION

### Example 1

### Synthesis of Compound 20 in Table 1

Compound 20 was prepared using the following reaction scheme:

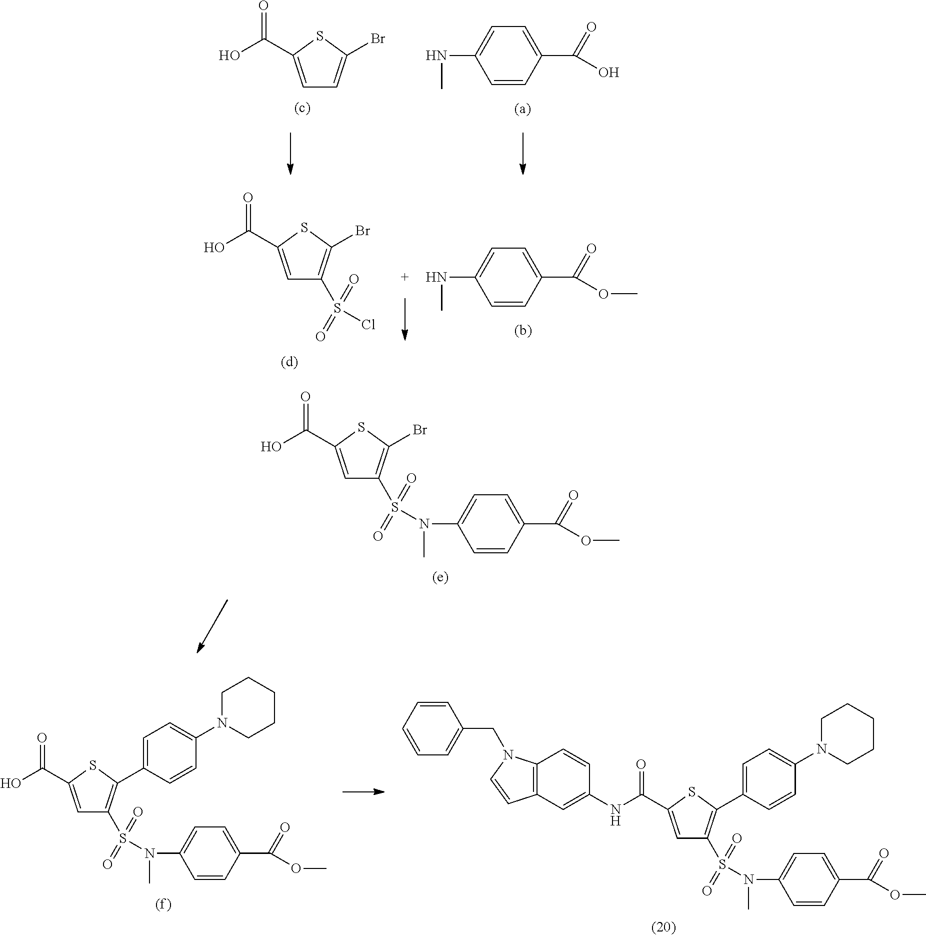

Step 1—Synthesis of (b) (Methyl 4-(methylamino)benzoate

Thionyl chloride (0.72 mL, 9.92 mmol, 1.50 eq.) was added to a stirred solution of 4-methyl amino benzoic acid (a) (1.0 g, 6.62 mmol, 1 eq.) in anhydrous methanol (0.136 mmol) at 0° C. and under N₂. After 5 minutes in an ice bath, the reaction mixture was refluxed for 3 hours. The solution was quenched with NaHCO₃at 0° C. and extracted with ethyl acetate (10 mL per 1 mmol of acid). The organic layer was washed with brine, dried with anhydrous Na2SO4 and the product, (b) was purified by column chromatography (eluent DCM) as a white solid with 85% yield.

FT-IR (Neat): v (cm⁻¹)=3389, 2946, 2359, 1684, 1596, 1436, 1273, 1169, 833, 770; ¹H-NMR (400 MHz, CDCl₃): δ ppm 7.85-7.91 (m, 2H), 6.52-6.59 (m, 2H), 4.19 (br. s, 1H), 3.86 (s, 3H), 2.89 (s, 3H); ¹³C-NMR (100 MHz, CDCl₃): δ 167.67, 153.18, 131.80, 131.80, 118.56, 111.39, 111.39, 51.79, 30.45; HRMS-ESI (m/z): calcd. for C₉H₁₁NO₂=165.0790, found=165.0715.

Step 2—Synthesis of (d) (5-Bromo-4-(chlorosulfonyl)thiophene-2-carboxylic acid)

Chlorosulfonic acid (3.86 mL, 57.96 mmol, 12 eq.) was added dropwise to a vigorously stirred solution of 5-methyl-2-thiophine carboxylic acid (c) (1 g, 4.83 mmol, 1 eq.) at −5° C. and under N₂. The solution was stirred for 40 hours at room temperature and quenched by pouring 250 g of ice into it very slowly. The product, (d), was precipitated out, collected by filtration and dried overnight over CaCl₂ as a white solid with 85% yield.

FT-IR (Neat): v (cm⁻¹)=3091, 2361, 1686, 1522, 1373, 1253, 1177, 1013, 874, 750; ¹H-NMR (400 MHz, CDCl₃): δ ppm 9.02 (s, 1H, COOH), 8.22 (s, 1H, H³); ¹³C-NMR (100 MHz, CDCl₃): δ 160.5 (COOH), 142.5, 134.0, 133.6, 127.7; HRMS-ESI (m/z): calcd. for C₅H₂BrClO₄S₂=303.8266, found=305.8256.

Step 3—Synthesis of (e) (5-bromo-4-(N-(4-(methoxycarbonyl)phenyl)-N-methylsulfamoyl)thiophene-2-carboxylic acid)

DMAP (99.96 mg, 1818.19 μmol, 0.5 eq.) was added to a stirred solution of (b) (500 mg, 1.64 mmol, 1 eq.) at room temperature in MeOH and under N₂. After 30 minutes, (d) (810.93 mg, 4.91 mmol, 3 eq.) was added. The solution was left stirred for 5 hrs at room temperature. Saturated brine was added to quench the reaction and the mixture was extracted with DCM (×3). The organic phase was dried by anhydrous MgSO₄ and the product (e) was isolated as a white solid by column chromatography with DCM as an eluent. (Yield 80%) The structure of the product was elucidated by NMR and mass spectroscopy. The presence of a single regioisomer was confirmed by NMR including HMBC and NOESY.

FT-IR (Neat): v (cm⁻¹)=1705, 1604, 1408, 1277, 1175, 1059, 879, 772, 700; ¹H-NMR (400 MHz, Methanol-d): δ ppm 7.96-8.00 (m, 2H), 7.46 (s, 1H), 7.36-7.40 (m, 2H), 3.89 (s, 3H), 3.36 (s, 3H); ¹³C-NMR (100 MHz, CDCl₃): δ 164.63, 159.75, 156.79, 140.83, 139.48, 134.76, 131.74, 131.56, 127.57, 121.89, 117.23, 115.48, 56.72, 27.26; HRMS-ESI (m/z): calcd. for C₁₄H₁₂BrNO₆S₂=432.9289, found=432.9381.

Step 4—Synthesis of (f) (4-(N-(4-(methoxycarbonyl)phenyl)-N-methylsulfamoyl)-5-(4-(piperidin-1-yl)phenyl)thiophene-2-carboxylic acid)

Compound (e) (50 mg, 115.13 μmol, 1 eq.) was taken in a microwave vial along with a solvent mixture (3 mL) of ethanol, toluene and water (9:3:1). 1-[4-(4,4,5,5-Tetramethyl-1,3,2-dioxaborolan-2-yl)phenyl]piperidine (39.68 mg, 138.16 μmol, 1.2 eq.) and K₂CO₃ (47.74 mg, 345.40 μmol, 3 eq.) were added to the solution. Tetrakis (triphenylphosphine) palladium (13.30 mg, 11.51 μmol, 0.1 eq.) was then added under N₂ and incubated in microwave irradiation at 100° C. for 20 minutes. The solution was quenched with water. The pH of the water layer was 11, 0.025 N HCl was added to the solution to make the pH 2/3; then the water layer was extracted with DCM (×3). The combined organic layer was dried with anhydrous MgSO₄ and the product, (f) was purified by column chromatography (Et2O with 1% methanol) as a yellow solid with 60% yield.

FT-IR (Neat): v (cm⁻¹)=2935, 2360, 1714, 1603, 1439, 1279, 1114, 885, 773, 698; ¹H-NMR (400 MHz, METHANOL-d): δ ppm 7.86 (s, 1H), 7.77 (d, J=8.8 Hz, 2H), 7.23 (d, J=8.8 Hz, 2H), 7.07 (d, J=8.8 Hz, 2H), 6.76 (d, J=8.8 hz, 2H), 3.88 (s, 3H), 3.19-3.24 (m, 4H), 3.05 (s, 3H), 1.64-1.72 (m, 6H); ¹³C-NMR (100 MHz, METHANOL-d): δ 163.89, 162.23, 150.66, 143.52, 142.11, 135.50, 134.19, 131.23, 130.67, 128.59, 128.34, 126.00, 119.84, 119.01, 113.60 (2C), 110.96, 110.61, 61.10 (2C), 52.94, 28.99, 25.81, 25.08, 22.41; HRMS-ESI (m/z): calcd. for C₂₅H₂₆N₂O₆S₂=514.1232, found=514.1296.

Step 5—Synthesis of compound 20 in Table 1 (methyl 4-((5-((1-benzyl-1H-indol-5-yl)carbamoyl)-N-methyl-2-(4-(piperidin-1-yl)phenyl)thiophene)-3-sulfonamido)benzoate)

Compound (f) (20 mg, 38.86 μmol, 1 eq.) was taken in a flask along with DMF as a solvent (approx. 1.5 mL per 1 mmol). 1-Benzyl-1H-indol-5-ylamine (10.37 mg, 46.64 μmol, 1.2 eq.), EDC hydrochloride (14.90mg, 77.73 μmol, 2 eq.), and DMAP (11.87 mg, 97.16 μmol, 2.5 eq.) were added respectively at room temperature and stirred under N₂. After 1.5 hours, the reaction was finished. The solution was quenched with water and then extracted with ethyl acetate. The organic layer was dried with anhydrous MgSO₄ and the product, (20) was purified by column chromatography (eluent hexane: ethyl acetate) as a yellow solid with 65% yield.

FT-IR (Neat): v (cm−1)=2356, 1738, 1540, 1361, 1217, 796, 628; ¹H NMR (400 MHz, METHANOL-d): δ ppm 8.23 (s, 1H), 7.93 (s, 1H), 7.82-7.87(m, 2H), 7.37 (s, 1H), 7.37 (s, 2H), 7.32-7.35 (m, 2H), 7.30 (d, J=7.07 Hz, 3H), 7.20 (d, J=7.07 Hz, 2H), 7.16 (d, J=8.84 Hz, 2H), 6.82 (d, J=8.84 Hz, 2H), 6.57 (d, J=3.03 Hz, 1H), 5.54 (s, 2H), 3.94 (s, 3H), 3.55 (q, J=7.07 Hz, 4H), 3.16 (s, 3H), 1.67-1.81 (m, 6H); ¹³C NMR (100 MHz, CDCl₃): δ 167.97, 156.94, 147.41, 147.02, 144.67, 139.71, 139.46, 137.87, 136.19, 132.10, 131.63, 130.37 (2C), 129.70 (2C), 128.82, 128.27, 125.34, 124.21, 123.17, 122.35, 120.57, 120.49, 119.65, 118.42, 114.41 (2C), 112.59 (2C), 107.85, 106.43, 104.12, 67.87, 61.57, 56.59, 55.78, 37.15, 25.57, 23.26, 20.02; HRMS-ESI (m/z): calcd. for C₄₀H₃₈N₄O₅S₂=718.2284, found=718.2367.

### Example 2

Synthesis of Compounds 21 to 40 in Table 1

Compounds 21 to 40 were prepared using the following reaction scheme.

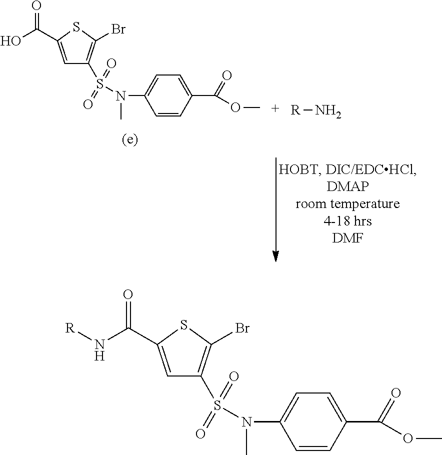

A large-scale synthesis of the key intermediate (e) was carried out using the procedure described in Example 1. A total of 6.5 g (e) was synthesized, and it was then coupled to the 19 amine fragments selected by an in silico study.

### Example 2(a)

### Synthesis of Compound 21 (methyl 4-((2-bromo-N-methyl-5-((4-(piperidin-1-yl)phenyl)carbamoyl)thiophene)-3-sulfonamido)benzoate)

Compound (e) (184.79 mg, 425.51 μmol, 1.5 eq.) was taken in a flask along with DMF as a solvent (approx. 1.5 mL per 1 mmol). 4-(Piperidin-1-yl) aniline (50 mg, 283.67 μmol, 1 eq.), EDCI (108.76 mg, 567.35, 2 eq.), and DMAP (86.64 mg, 709.19 μmol, 2.5 eq.) were added respectively at room temperature and stirred under N₂. After 1.5 hours, the reaction was finished. The reaction mixture was passed through a SCX-2 cartridge (5.0 gm) and the cartridge was washed with DCM (3×) and DMF (3×) twice and finally with MeOH (2×). The product compound 21 was released from the cartridge using 5.0 ml 2 M NH₃ in MeOH and concentrated in vacuo to obtain a brown solid (yield 40%).

FT-IR (Neat): v (cm⁻¹)=3368, 2918, 2849, 1709, 1644, 1602, 1536, 1431, 1419, 1344, 1325, 1278, 1128; ¹H-NMR (400 MHz, CDCl₃): δ ppm 8.00-8.03 (m, 2H), 7.42 (d, J=8.84 Hz, 3H), 7.31 (d, J=8.59 Hz, 2H), 6.90-6.93 (m, 2H), 3.92 (s, 3H), 3.39 (s, 3H), 3.13-3.17 (m, 4H), 1.68-1.75 (m, 6H); ¹³C-NMR (100 MHz, CDCl₃): δ 166.13, 163.16, 145.79, 144.67, 142.86, 138.70, 133.68, 130.59, 129.18, 126.08, 122.03, 121.64 (2C), 121.24, 116.78, 115.62, 115.20, 114.73, 52.31 (2C), 50.69, 30.92, 25.74, 24.22 (2C); HRMS-ESI (m/z): calcd. for C₂₅H₂₆BrN₃O₅S₂=591.0497, found=591.0570.

### Example 2(b)

### Synthesis of Compound 22 (methyl 4-((2-bromo-N-methyl-5-((3-morpholinophenyl)carbamoyl)thiophene)-3-sulfonamido)benzoate)

Compound (e) (146.20 mg, 336.64 μmol, 1.5 eq.) was taken in a flask along with DMF as a solvent (approx. 1.5 mL per 1 mmol). 3-Morpholinoaniline (40 mg, 224.43 μmol, 1 eq.), HOBT (60.65 mg, 448.86 μmol, 2 eq.), and DIC (60.82 μL, 392.75 μmol, 1.75 eq.) were added respectively at room temperature and stirred under N₂. After 18 hours, the reaction was finished. The solution was quenched with water and then extracted with ethyl acetate. The organic layer was combined and dried with MgSO₄ and the product, 22, was purified by column chromatography (eluent Hexane: Ether 1:1) as a white solid with 83% yield.

FT-IR (Neat): v (cm⁻¹)=3337, 2968, 2872, 1723, 1658, 1606, 1548, 1497, 1430, 1361, 1272, 1249, 1167, 1146, 1129; ¹H-NMR (400 MHz, CDCl₃): δ ppm 8.41 (s, 1H), 7.99-8.02 (m, 2H), 7.66 (s, 1H), 7.39-7.41 (m, 1H), 7.30-7.33 (m, 2H), 7.24 (t, J=8.18 Hz, 1H), 7.00 (dd, J=7.93, 1.38 Hz, 1H), 6.71 (dd, J=8.18, 2.14 Hz, 1H), 3.91 (s, 3H), 3.86 (d, J =4.78 Hz, 4H), 3.38 (s, 3H), 3.16-3.20 (m, 4H); ¹³C-NMR (100 MHz, CDCl₃): δ 171.19, 160.57, 155.42, 144.63, 143.63, 138.13, 136.56, 134.05, 130.76, 130.56 (2C), 129.10, 126.01, 123.64, 114.29 (2C), 112.17, 109.54, 66.81, 66.63, 52.32, 49.04, 42.26, 23.43; HRMS-ESI (m/z): calcd. for C₂₄H₂₄BrN₃O₆S₂=593.0290, found=593.0361.

### Example 2c

### Synthesis of Compound 23 (methyl 4-((2-bromo-N-methyl-5-((4-(morpholinomethyl)phenyl)carbamoyl)thiophene)-3-sulfonamido)benzoate)

Compound (e) (169.41 mg, 390.10 μmol, 1.5 eq.) was taken in a flask along with DMF as a solvent (approx. 1.5 mL per 1 mmol). 4-(Morpholinomethyl)aniline (50 mg, 260.07 μmol, 1 eq.), HOBT (70.28 mg, 520.14 μmol, 2 eq.), and DIC (71.26 μL, 455.12 μmol, 1.75 eq.) were added respectively at room temperature and stirred under N₂. After 2 hours, the reaction was finished. The reaction mixture was passed through a SCX-2 cartridge (sorbent mass should be 10 times of the product mass) (5.0 gm) and the cartridge was washed with DCM (3×) and

DMF (3×) twice and finally MeOH (2×). The product 23, along with a small amount of impurities, was released from the cartridge using 5.0 ml 2M NH₃ in MeOH and concentrated in vacuo. To remove the impurities, the solid was purified through column chromatography using silica gel and DCM; Ethyl Acetate 4:1 as an eluent. The product 23 was obtained as a yellow solid with 86% yield.

FT-IR (Neat): v (cm⁻¹)=3610, 3365, 2889, 1716, 1634, 1600, 1546, 1410, 1337, 1321, 1277, 1230, 1184, 1146; ¹H-NMR (400 MHz, CDCl₃): δ ppm 7.98-8.02 (m, 2H), 7.49 -7.56 (m, 3H), 7.27-7.35 (m, 4H), 3.91 (s, 3H), 3.67-3.73 (m, 4H), 3.48 (s, 2H), 3.39 (s, 3H), 2.41-2.46 (m, 4H); ¹³C-NMR (100 MHz, CDCl₃): δ 166.11, 157.70, 144.59, 141.09, 136.62, 135.88, 134.89, 131.18, 130.60, 129.89, 129.23, 127.84, 126.08, 122.51, 120.46, 120.30, 111.57, 111.02, 67.00 (2C), 62.83, 53.58 (2C), 52.34, 30.92; HRMS-ESI (m/z): calcd. for C₂₅H₂₆BrN₃O₆S₂=607.0446, found=607.0520.

### Example 2d

### Synthesis of Compound 24 (methyl 4-((2-bromo-N-methyl-5-((4-morpholinobenzyl)carbamoyl)thiophene)-3-sulfonamido)benzoate)

Compound (e) (169.41 mg, 390.10 μmol, 1.5 eq.) was taken in a flask along with DMF as a solvent (approx. 1.5 mL per 1 mmol). (4-Morpholinophenyl)methanamine (50 mg, 260.07 μmol, 1 eq.), HOBT (70.28 mg, 520.14 μmol, 2 eq.), and DIC (70.47 μL, 455.12 μmol, 1.75 eq.) were added respectively at room temperature and stirred under N₂. After 18 hours, the reaction was finished. The solution was quenched with water and then extracted with ethyl acetate. The organic layer was combined and dried with anhydrous MgSO₄ and the product, 24 was purified by column chromatography (eluent Hexane: Ether 1:1) as a white solid with 98% yield.

FT-IR (Neat): v (cm⁻¹)=3378, 2919,1546, 1375, 1276, 1253, 1192, 1168, 1125, 715, 702, 635, 606, 571 ;¹H-NMR (400 MHz, CDCl₃): δ ppm 7.98-8.01 (m, 2H), 7.28-7.31 (m, 3H), 7.22 (d, J=8.81 Hz, 2H), 6.89 (d, J=8.81 Hz, 2H), 6.27 (t, J=5.54 Hz, 1H), 4.47 (d, J=5.54 Hz, 2H), 3.92 (s, 3H), 3.85-3.89 (m, 4H), 3.36 (s, 3H), 3.14-3.18 (m, 4H); ¹³C-NMR (100 MHz, CDCl₃): δ 165.55, 158.15, 149.80, 144.69, 140.34, 137.85, 130.57, 129.26, 128.78, 127.75, 126.48, 126.04, 122.81, 120.50, 118.46, 115.86, 113.18, 112.60, 66.82 (2C), 53.58 (2C), 49.19, 44.78, 29.50; HRMS-ESI (m/z): calcd. for C₂₅H₂₆BrN₃O₆S₂=607.0552, found=607.0518.

### Example 2e

### Synthesis of Compound 29 (methyl 4-((2-bromo-N-methyl-5-((4-(2-morpholinoethoxy)phenyl)carbamoyl)thiophene)-3-sulfonamido)benzoate)

Compound (e) (117.22 mg, 269.93 μmol, 1.5 eq.) was taken in a flask along with DMF as a solvent (approx. 1.5 mL per 1 mmol). 4-(2-Morpholinoethoxy)aniline (40 mg, 179.95 μmol, 1 eq.), EDCI.HCl (68.99 mg, 359.90 μmol, 2 eq.), and DMAP (54.96 mg, 449.87 μmol, 2.5 eq.) were added respectively at room temperature and stirred under N₂. After 18 hours, the reaction was quenched with water and then extracted with ethyl acetate. The organic layer was combined and dried with anhydrous MgSO₄. The product 29 was purified by column chromatography using silica gel and ethyl acetate and methanol (up to 3%) as an eluent. The product 29 was obtained as a white solid with 55% yield.

FT-IR (Neat): v (cm⁻¹)=3337, 2968, 1695, 1652, 1612, 1515, 1457, 1436, 1412, 1376, 1325, 1228, 1168; ¹H-NMR (400 MHz, CDCl₃): δ ppm 7.98-8.03 (m, 2H), 7.79 (s, 1H), 7.43-7.50 (m, 3H), 7.28-7.34 (m, 2H), 6.91 (d, J=9.09 Hz, 2H), 4.09-4.15 (m, 2H), 3.92 (s, 3H), 3.72-3.77 (m, 4H), 3.39 (s, 3H), 2.81 (t, J=5.68 Hz, 2H), 2.55-2.62 (m, 4H); ¹³C-NMR (100 MHz, CDCl₃): δ 166.13, 160.73, 153.99, 139.37, 138.70, 136.69, 135.07, 130.60, 129.97, 129.20, 126.97, 126.08, 122.30 (2C), 115.19 (2C), 111.45, 110.22, 66.90 (2C), 66.12, 57.63, 54.10 (2C), 52.34, 38.44; HRMS-ESI (m/z): calcd. for C₂₆H₂₈BrN₃O₇S₂637.0552, found=637.0623.

### Example 2f

### Synthesis of Compound 26 in-(methyl 4-((2-bromo-N-methyl-5-((4-(piperidin-1-yl)benzyl)carbamoyl)thiophene)-3-sulfonamido)benzoate)

Compound (e) (102.70 mg, 236.49 μmol, 1.5 eq.) was taken in a flask along with DMF as a solvent (approx. 1.5 mL per 1 mmol).(4-(piperidin-1-yl)phenyl)methanamine (30 mg, 157.66 μmol, 1 eq.), HOBT (42.61 mg, 315.32 μmol, 2 eq.), and DIC (42.72 μL, 275.90 μmol, 1.75 eq.) were added respectively at room temperature and stirred under N₂. After 18 hours, the reaction was finished. The reaction mixture was passed through a SCX-2 cartridge (5.0 gm) and the cartridge was washed with DCM (3×) and DMF (3×) twice and finally MeOH (2×). The product 26, along with small amount of impurities, was released from the cartridge using 5.0 ml 2M NH₃ in MeOH and concentrated in vacuo. To remove the impurities, the solid was purified through column chromatography using silica gel and ether as an eluent. The product 26 was obtained as a white solid with 95% yield.

FT-IR (Neat): v (cm⁻¹)=3374, 2946, 2919, 1694, 1650, 1602, 1546, 1513, 1457, 1444, 1387, 1371, 1323, 1283, 1235, 1191; ¹H-NMR (400 MHz, CDCl₃): δ ppm 8.00 (d, J=8.8 hz, 2H), 7.28-7.32 (m, 3H), 7.19 (d, J=8.8 hz, 2H), 6.91 (d, J=8.6 hz, 2H), 6.16 (t, J=5.16 Hz, OH), 4.46 (d, J=5.54 Hz, 2H), 3.35 (s, 3H), 3.92 (s, 3H), 3.14-3.19 (m, 4H), 1.65-1.75 (m, 4H), 1.59-1.62 (m, 2H); ¹³C-NMR (100 MHz, CDCl₃): δ 166.49, 159.52, 152.26, 144.99, 140.75, 136.77, 130.86, 129.45, 129.36, 127.75, 127.24, 126.33, 122.03 (2C), 116.77 (2C), 113.81, 112.57, 52.62 (2C), 50.69, 44.18, 31.04, 26.00, 24.56, 23.80; HRMS-ESI (m/z): calcd. for C₂₆H₂₈BrN₃O₅S₂=605.0654, found=605.0727.

### Example 2g

### Synthesis of Compound 27 1 (methyl 4-(5-bromo-4-(N-(4-(methoxycarbonyl)phenyl)-N-methylsulfamoyl)thiophene-2-carboxamido)benzoate)

Compound (e) (550 mg, 1.27 mmol, 3 eq.) was taken in a flask along with DMF as a solvent (approx. 1.5 mL per 1 mmol). Methyl 4-aminobenzoate (70.57 mg, 422.15 μmol, 1 eq.), HOBT (114.09 mg, 844.31 μmol, 2 eq.), and DIC (114.40 μL, 738.77 μmol, 1.75 eq.) were added respectively at room temperature and stirred under N₂. After 8 days, the reaction was finished. The reaction mixture was quenched with water and then extracted with ethyl acetate.

The organic layer was combined and dried with anhydrous MgSO₄ and concentrated in vacuo. The product, 27 was purified by column chromatography (eluent hexane:ether 1:1) as a brown solid with 70% yield.

FT-IR (Neat): v (cm⁻¹)=3363, 2946, 1716, 1671, 1600, 1532, 1510, 1449, 1415, 1404, 1343, 1319, 1248, 1153; ¹H-NMR (400 MHz, CDCl₃): δ ppm 10.89 (s, 1H), 8.01-8.04 (m, 2H), 7.84 (d, J=8.81 Hz, 1H), 7.71 (s, 1H), 7.47 (s, 1H), 7.32-7.35(m, 2H), 7.28 (d, J=2.01 Hz, 1H), 7.12 (dd, J=8.69, 2.14 Hz, 1H), 3.96 (s, 3H), 3.93 (s, 3H), 3.41 (s, 3H); ¹³C-NMR (100 MHz, CDCl₃): δ 172.47, 172.04, 162.66, 144.98, 143.31, 139.36, 137.69, 135.52, 131.73, 130.01, 128.46, 128.26, 127.84, 126.16, 124.94, 119.94, 119.09, 115.23, 113.58, 55.27, 52.56, 33.09; HRMS-ESI (m/z): calcd. for C₂₂Hl₉BrN₂O₇S₂=565.9817, found=565.0281.

### Example 2 h

### Synthesis of Compound 28 (methyl 4-((2-bromo-N-methyl-5-((3-(piperidin-1-yl)phenyl)carbamoyl)thiophene)-3-sulfonamido)benzoate)

Compound (e) (184.79mg, 425.51 μmol, 1.5 eq.) was taken in a flask along with DMF as a solvent (approx. 1.5 mL per 1 mmol). 3-(piperidin-1-yl)aniline (50 mg, 283.67 μmol, 1 eq.),

HOBT (76.66 mg, 567.35 μmol, 2 eq.), and DIC (76.87 μL, 496.43 μmol, 1.75 eq.) were added respectively at room temperature and stirred under N₂. After 18 hours, the reaction was finished. The reaction mixture was quenched with water and then extracted with ethyl acetate. The organic layer was combined and dried with anhydrous MgSO₄ and was concentrated in vacuo. To remove the impurities, the crude solid was purified through column chromatography using silica gel and hexane:ether 8:2 as an eluent. The product 28 was obtained as a brown solid with 95% yield.

FT-IR (Neat): v (cm⁻¹)=2937, 2849, 1715, 1669, 1605, 1543, 1496, 1404, 1363, 1352, 1325, 1310, 1275, 1253, 1208, 1177, 1112; ¹H-NMR (400 MHz, CDCl₃): δ ppm 8.40 (s, OH), 7.83-7.90 (m, 2H), 7.55 (s, 1H), 7.23 (s, 1H), 7.13-7.20 (m, 2H), 7.04-7.13 (m, 1H), 6.87-6.93 (m, 1H), 6.63 (dd, J=8.31, 2.01Hz, 1H), 3.81 (s, 3H), 3.23 (s, 3H), 3.02-3.09 (m, 4H), 1.53-1.62 (m, 4H), 1.43-1.51 (m, 2H); ¹³C-NMR (100 MHz, CDCl₃): δ 166.43, 158.22, 152.98, 144.79, 141.85, 138.06, 136.44, 130.80 (2C), 129.66, 129.28, 126.25, 122.67, 113.37 (2C), 111.24 (2C), 108.58, 52.61 (2C), 50.50, 38.57, 25.93 (2C), 24.50; HRMS-ESI (m/z): calcd. for C₂₅H₂₆BrN₃O₅S₂=591.0497, found=591.0574.

### Example 2i

### Synthesis of Compound 25—(methyl 4-((2-bromo-N-methyl-5-((4-(2-morpholinoethoxy)benzyl)carbamoyl)thiophene)-3-sulfonamido)benzoate)

Compound (e) (137.83 mg, 317.38 μmol, 1.5 eq.) was taken in a flask along with DMF as a solvent (approx. 1.5 mL per 1 mmol). (4-(2-morpholinoethoxy)phenyl)-methanamine (50 mg, 211.59 μmol, 1 eq.), HOBT (57.18 mg, 423.17 μmol, 2 eq.), and DIC (57.34 μL, 370.28 μmol, 1.75 eq.) were added respectively at room temperature and stirred under N₂. After 18 hours, the reaction was completed and the reaction mixture was quenched with water and then extracted with ethyl acetate. The organic layer was combined and dried with anhydrous MgSO₄ and concentrated in vacuo, The crude solid was purified through column chromatography using silica gel and DCM:ethyl acetate 8:2 as an eluent. The product 25 was obtained as a brown solid with 97% yield.

FT-IR (Neat): v (cm⁻¹)=3285, 2949, 1722, 1647, 1601, 1578, 1553, 1514, 1470, 1437, 1367, 1306, 1275, 1253, 1147, 1111; ¹H-NMR (400 MHz, CDCl₃): δ ppm 7.94 (d, J=8.56 Hz, 2H), 7.39 (s, 1H), 7.23 (d, J=8.56 Hz, 2H), 7.20 (d, J=8.81 Hz, 2H), 6.90 (br. s., 1H), 6.83 (d, J=8.56 Hz, 2H), 4.45 (d, J=5.54 Hz, 2H), 4.07 (t, J=5.67Hz, 2H), 3.89 (s, 3 H), 3.67-3.72 (m, 4H), 3.30 (s, 3H), 2.77 (t, J=5.79 Hz, 2H), 2.52-2.57 (m, 4H); ¹³C-NMR (100 MHz, CDCl₃): δ 166.17, 159.52, 158.34, 144.63, 140.68, 136.27, 130.52, 129.69, 129.42, 129.00, 127.67, 125.99, 121.86, 121.76, 114.84 (2C), 113.8 (2C), 66.89 (2C), 65.77, 57.60, 54.06 (2C), 52.37, 43.55, 38.31; HRMS-ESI (m/z): calcd. for C₂₇H₃₀BrN₃O₇S₂=651.0707, found=651.0782.

### Example 2j

### Synthesis of Compound 30 (methyl 4-((2-bromo-N-methyl-5-((6-morpholinopyridin-3-yl)carbamoyl)thiophene)-3-sulfonamido)benzoate

Compound (e) (145.39 mg, 334.79 μmol, 1.5 eq.) was taken in a flask along with DMF as a solvent (approx. 1.5 mL per 1 mmol). 6-Morpholinopyridin-3-amine (40 mg, 223.19 μmol, 1 eq.), HOBT (60.32 mg, 446.38 μmol, 2 eq.), and DIC (60.48 μL, 390.58 μmol, 1.75 eq.) were added respectively at room temperature and stirred under N₂. After 18 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and hexane:ether 8:2 as eluent. The product, 30, was obtained as a yellow solid with 95% yield.

FT-IR (Neat): v (cm⁻¹)=3337, 2968, 1720, 1655, 1612, 1566, 1360, 1324, 1277, 1244, 1168, 1129, 1117; ¹H-NMR (400 MHz, CDCl₃): δ ppm 8.29 (br. s., 1H), 7.99-8.03 (m, 2H), 7.70-7.73 (m, 1H), 7.35-7.47 (m, 2H), 7.31-7.33 (m, 1H), 7.29-7.31 (m, 1H), 6.66 (d, J=9.32 Hz, 1H), 3.92 (s, 3H), 3.82-3.85 (m, 4H), 3.47-3.52 (m, 4H), 3.38 (s, 3H); ¹³C-NMR (100 MHz, CDCl₃): δ 164.08, 159.47, 155.03, 150.12, 144.54, 142.45, 137.49, 135.06, 130.58, 128.16, 127.44, 125.88, 122.76, 121.92, 114.36, 112.61, 104.05, 66.67 (2C), 52.37, 42.41 (2C), 30.51; HRMS-ESI (m/z): calcd. for C₂₃H₂₃BrN₄O₆S₂=594.0242, found=594.0322.

### Example 2k

### Synthesis of Compound 31 (methyl 4-((2-bromo-N-methyl-5-((4-(morpholinomethyl)benzyl)carbamoyl)thiophene)-3-sulfonamido)benzoate)

Compound (e) (157.89 mg, 363.58 μmol, 1.5 eq.) was taken in a flask along with DMF as a solvent (approx. 1.5 mL per 1 mmol). (4-(Morpholinomethyl)phenyl)methanamine (50 mg, 242.38 μmol, 1 eq.), HOBT (65.50 mg, 484.77 μmol, 2 eq.), and DIC (66.41 μL, 424.17 μmol, 1.75 eq.) were added respectively at room temperature and stirred under N₂. After 18 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and eluent 100% ethyl acetate. The product, 31, was obtained as a white solid with 72% yield.

FT-IR (Neat): v (cm⁻¹)=3297, 2183, 1718, 1657, 1555, 1436, 1409, 1361, 1347, 1276, 1184, 1146, 1102; ¹H-NMR (400 MHz, CDCl₃): δ ppm 7.98-8.02 (m, 2H), 7.31-7.34 (m, 3H), 7.29-7.31 (m, 1H), 7.27-7.29 (m, 1H), 7.25 (s, 1H), 6.35 (t, J=5.54 Hz, 1H), 4.54 (d, J=5.54 Hz, 2H), 3.92 (s, 3H), 3.68-3.72 (m, 4H), 3.49 (s, 2H), 3.36 (s, 3H), 2.40-2.47 (m, 4H); ¹³C-NMR (100 MHz, CDCl₃): δ 166.12, 159.45, 144.57, 140.42, 137.54, 136.32, 136.12, 130.50 (2C), 129.62 (2C), 129.03, 127.98 (2C), 127.57, 126.00 (2C), 121.91, 66.89 (2C), 62.96, 53.51 (2C), 52.33, 43.83, 38.32; HRMS-ESI (m/z): calcd. for C₂₆H₂₈BrN₃O₆S₂=621.0603, found=621.0676.

### Example 21

### Synthesis of Compound 32 (methyl 4-((2-bromo-N-methyl-5-((4-(4-methylpiperazin-1-yl)benzyl)carbamoyl)thiophene)-3-sulfonamido)benzoate)

Compound (e) (222.11 mg, 511.45 μmol, 1.5 eq.) was taken in a flask along with DMF as a solvent (approx. 1.5 mL per 1 mmol). (4-((4-methylpiperazin-1-yl)methyl)phenyl)methanamine (70 mg, 340.97 μmol, 1 eq.), HOBT (92.14 mg, 681.93 μmol, 2 eq.), and DIC (93.43 μL, 596.69 μmol, 1.75 eq.) were added respectively at room temperature and under N₂. After 18 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and eluent ethyl acetate and methanol (up to 2%). The product, 32, was obtained as a brown solid with 51% yield.

FT-IR (Neat): v (cm⁻¹)=2941, 2797, 1720, 1634, 1548, 1514, 1451, 1435, 1290, 1275, 1174, 1142, 1112; ¹H-NMR (400 MHz, CDCl₃): δ ppm 7.98 (d, J=9.1 Hz, 2H), 7.33 (s, 1H), 7.25-7.30 (m, 2H), 7.21 (d, J=8.6 Hz, 2H), 6.89 (d, J=8.6 Hz, 2H), 6.40 (t, J=4.78 Hz, NH), 4.46 (d, J=5.54 Hz, 2H), 3.91 (s, 3H), 3.33 (s, 3H), 3.18-3.24 (m, 4H), 2.55-2.62 (m, 4H), 2.36 (s, 3H); ¹³C-NMR (100 MHz, CDCl₃): δ 166.20, 159.32, 151.02, 144.67, 140.53, 136.40, 130.56, 129.23, 129.05, 127.84, 127.52, 127.33, 126.03 (2C), 116.11 (2C), 115.83 (2C), 55.01 (2C), 52.35 (2C), 48.87, 46.12, 43.76, 30.36; HRMS-ESI (m/z): calcd. for C₂₆H₂₉BrN₄O₅S₂=621.0763, found=621.0828.

### Example 2m

### Synthesis of Compound 33 (methyl 4-((2-bromo-N-methyl-5-((4-(piperidin-1-ylmethyl)benzyl)carbamoyl)thiophene)-3-sulfonamido)benzoate)

Compound (e) (478.24 mg, 1.10 mmol, 1.5 eq.) was taken in a flask along with DMF as a solvent (approx. 1.5 mL per 1 mmol). (4-(piperidin-1-ylmethyl)phenyl)methanamine (150 mg, 734.15 μmol, 1 eq.), HOBT (198.41 mg, 1.47 mmol, 2 eq.), and DIC (201.17 μL, 1.28 mmol, 1.75 eq.) were added respectively at room temperature and under N₂. After 18 hours, the reaction was finished. The solution was quenched with water and then extracted with ethyl acetate. The organic layer was combined and dried with anhydrous MgSO₄ and concentrated in vacuo. The crude mixture was passed through a SCX-2 cartridge (5.0 gm) and the cartridge was washed with DCM (3×) and DMF (3×) twice and finally MeOH (2×). The pure product 33 was released from the cartridge using 5.0 ml 2M NH₃ in MeOH and concentrated in vacuo as a brown solid with 90% yield.

FT-IR (Neat): v (cm⁻¹)=2937, 2797, 1717, 1606, 1546, 1509, 1435, 1407, 1363, 1322, 1281, 1185, 1112; ¹H-NMR (400 MHz, CDCl₃): δ ppm 7.96-8.01 (m, 2H), 7.54 (s, 1H), 7.49 (t, J=5.79 Hz, 1H), 7.24-7.32 (m, 6H), 4.53 (d, J=5.79 Hz, 2H), 3.93 (s, 3H), 3.46 (s, 2H), 3.33 (s, 3H), 2.38 (br. s., 4H), 1.54-1.62 (m, 4H), 1.42-1.48 (m, 2H); ¹³C-NMR (100 MHz, CDCl₃): δ 166.15, 159.56, 144.59, 140.56, 137.17, 136.77, 136.21, 131.44, 130.47, 129.80, 129.21, 128.92, 127.86 (2C), 126.45, 125.94, 121.78, 103.19, 63.10, 54.24, 52.77, 52.30, 38.27, 31.93, 25.50, 24.03, 23.39; HRMS-ESI (m/z): calcd. for C₂₇H₃₀BrN₃O₅S₂=619.0810, found=619.0881.

### Example 2n

### Synthesis of Compound 34 (methyl 4-((2-bromo-N-methyl-5-((4-((5-methyl-1H-benzo[d]imidazol-2-yl)methyl)phenyl)carbamoyl)thiophene)-3-sulfonamido)benzoate)

Compound (e) (140.00 mg, 322.38 μmol, 1.5 eq.) was taken in a flask along with DMF as a solvent (approx. 1.5 mL per 1 mmol). 4-(5-methyl-1H-benzoimidazol-2-ylmethyl)phenylamine (51 mg, 214.92 μmol, 1 eq.), HOBT (58.08 mg, 429.84 μmol, 2 eq.), and DIC (58.24 μL, 376.11 μmol, 1.75 eq.) were added respectively at room temperature and stirred under N₂. After 18 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and hexane:ethyl acetate 2:8 as eluent. The product, 34 was obtained as a white solid with 48% yield.

FT-IR (Neat): v (cm⁻¹)=2920, 2852, 1716, 1644, 1604, 1572, 1552, 1537, 1513, 1503, 1435, 1416, 1370, 1360, 1331, 1310, 1244, 1143; ¹H-NMR (400 MHz, CDCl₃): δ ppm 9.56 (br. s., NH), 7.91 (s, 1H), 7.31-7.39 (m, 2H), 7.21-7.28 (m, 4H), 7.11 (d, J=8.6 Hz, 2H), 7.03 (d, J=8.6 Hz, 1H), 6.72 (d, J=8.31 Hz, 2H), 4.02 (s, 2H), 3.85 (s, 3H), 3.24 (s, 3H), 2.34 (s, 3H); ¹³C-NMR (100 MHz, CDCl₃): δ 167.87, 166.32, 145.73, 144.77, 144.42, 139.09, 138.87, 136.55, 134.40, 132.81, 132.47, 132.09, 130.90, 130.55, 129.00, 128.85, 125.90, 123.87, 122.46, 121.77, 120.87, 115.52, 113.87, 108.03, 105.36, 60.49, 38.23, 29.77, 25.90; HRMS-ESI (m/z): calcd. for C₂₉H₂₅BrN₄O₅S₂=652.0450, found=652.0523.

### Example 2o

### Synthesis of Compound 35 (methyl 4-((5-((2-benzyl-1H-benzo[d]imidazol-5-yl)carbamoyl)-2-bromo-N-methylthiophene)-3-sulfonamido)benzoate

Compound (e) (150.00 mg, 345.40 μmol, 1.5 eq.) was taken in a flask along with DMF as a solvent (approx. 1.5 mL per 1 mmol). 2-benzyl-lh-benzimidazol-5-amine (51.41 mg, 230.27 μmol, 1 eq.), HOBT (62.23 mg, 460.54 μmol, 2 eq.), and DIC (62.50 μL, 402.97 μmol, 1.75 eq.) were added respectively at room temperature and stirred under N₂. After 18 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and hexane:ethyl acetate 3:7 as eluent. The product, 35, was obtained as a yellow solid with 76% yield.

FT-IR (Neat): v (cm⁻¹)=2920, 2850, 1715, 1698, 1693, 1655, 1603, 1561, 1555, 1535, 1492, 1453, 1433, 1412, 1327, 1297, 1157; ¹H-NMR (400 MHz, DMSO): δ ppm 12.27-12.32 (m, NH), 10.48 (d, J=14.86 Hz, NH), 8.21-8.24 (m, 1H), 7.94-7.98 (m, 2H), 7.93 (s, 1H), 7.51 (d, J=8.81 Hz, 1H), 7.43-7.46 (m, 2H), 7.39 (d, J=1.01 Hz, 1H), 7.32-7.33 (m, 4H), 7.21-7.27 (m, 1H), 4.16 (s, 2H), 3.84 (s, 3H), 3.34 (s, 3H);); ¹³C-NMR (100 MHz, DMSO): δ 168.12, 160.41, 146.88, 144.40, 143.11, 143.06, 138.55, 138.41, 136.73, 134.72, 134.27, 133.17, 131.80, 130.48 (2C), 130.19 (2C) 128.39, 127.68, 123.89, 123.56, 118.15, 112.91 (2C), 107.28, 53.13, 39.12, 36.50; HRMS-ESI (m/z): calcd. for C₂₈H₂₃BrN₄O₅S₂=638.0293, found=638.0355.

### Example 2p

### Synthesis of Compound 36 (methyl 4-((2-bromo-N-methyl-5-((1-methyl-1H-indol-5-yl)carbamoyl)thiophene)-3-sulfonamido)benzoate)

Compound (e) (225.00 mg, 518.10 μmol, 1.5 eq.) was taken in a flask along with DMF as a solvent (approx. 1.5 mL per 1 mmol). 5-aminol-n-methylindole (50.50 mg, 345.40 μmol, 1 eq.), HOBT (93.35 mg, 690.81 μmol, 2 eq.), and DIC (93.60 μL, 604.46 μmol, 1.75 eq.)

were added respectively at room temperature and stirred under N₂. After 18 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and hexane:ethyl acetate 7:3 as eluent. The product, 36, was a obtained as a white solid with 70% yield.

FT-IR (Neat): v (cm⁻¹)=2920, 2850, 2519, 1736, 1719, 1703, 1646, 1605, 1580, 1576, 1524, 1514, 1494, 1453, 1435, 1364, 1336, 1291, 1248, 1190; ¹H-NMR (400 MHz, CDCl₃): δ ppm 8.03 (d, J=8.81 Hz, 2H), 7.88 (s, 1H), 7.64 (br. s., 1H), 7.45 (br. s., 1H), 7.29-7.39 (m, 4H), 7.09 (d, J=3.02 Hz, 1H), 6.48 (d, J=3.02 Hz, 1H), 3.90-3.94 (m, 3H), 3.81 (s, 3H), 3.41 (s, 3H); ¹³C-NMR (100 MHz, CDCl₃): δ 168.48, 159.42, 145.18, 140.83, 138.10, 132.15 (2C), 131.86, 130.57, 130.40, 128.47, 128.03, 123.93, 123.41, 118.24, 117.45, 117.16, 110.34, 106.93, 102.57, 56.49, 36.50, 31.99; HRMS-ESI (m/z): calcd. for C₂₃H₂₀BrN₃O₅S₂=561.0028, found=561.0094.

### Example 2q

### Synthesis of Compound 37 (methyl 4-((2-bromo-N-methyl-5-((4-(pyrrolidin-1-ylmethyl)benzyl)carbamoyl)thiophene)-3-sulfonamido)benzoate

Compound (e) (170.00 mg, 391.46 μmol, 1.5 eq.) was taken in a flask along with DMF as a solvent (approx. 1.5 mL per 1 mmol). 4-pyrrolidin-1-ylmethyl-benzylamine (49.66 mg, 260.97 μmol, 1 eq.), HOBT (70.53 mg, 521.94 μmol, 2 eq.), and DIC (70.72 μL, 456.70 μmol, 1.75 eq.) were added respectively at room temperature and stirred under N₂. After 18 hours, the reaction was finished. The solution was quenched with water and then extracted with ethyl acetate. The organic layer was combined and dried with anhydrous MgSO₄ and concentrated in vacuo. The crude mixture was passed through a SCX-2 cartridge (5.0 gm) and the cartridge was washed with DCM (3×) and DMF (3×) twice and finally MeOH (2×). The pure product 37 was released from the cartridge using 5.0 ml 2M NH₃ in MeOH and concentrated in vacuo as a brown solid with 89% yield.

FT-IR (Neat): v (cm⁻¹)=2957, 2789, 1719, 1698, 1655, 1650, 1632, 1605, 1578, 1561, 1537, 1511, 1503, 1460, 1435, 1408, 1359, 1323, 1179; ¹H-NMR (400 MHz, METHANOL-d): δ ppm 7.98 (d, J=9.06 Hz, 2H), 7.73 (s, 1H), 7.35-7.44 (m, 3H), 7.27-7.35 (m, 3H), 4.47 (s, 2H), 3.89 (s, 3H), 3.71 (s, 2H), 3.37 (s, 3H), 2.61-2.67 (m, 4H), 1.82-1.87 (m, 4H); ¹³C-NMR (100 MHz, METHANOL-d): δ 168.21, 162.19, 146.98, 142.38, 139.59 (2C), 138.42, 138.03, 131.96, 131.40, 130.94, 130.58, 129.39 (2C), 127.81, 125.66, 123.70, 105.05, 61.33, 55.38, 53.82, 53.37, 44.75, 39.27, 24.60, 24.11; HRMS-ESI (m/z): calcd. for C₂₆H₂₈BrN₃O₅S₂=605.0654, found=605.0714.

### Example 2r

### Synthesis of Compound 38 (methyl 4-((2-bromo-N-methyl-5-((3-(2-methylthiazol-4-yl)phenyl)carbamoyl)thiophene)-3-sulfonamido)benzoate)

Compound (e) (170.00 mg, 391.46 μmol, 1.5 eq.) was taken in a flask along with DMF as a solvent (approx. 1.5 mL per 1 mmol). 3-2-methyl-1,3-thiazol-4-yl-aniline (49.66 mg, 260.97 μmol, 1 eq.), HOBT (70.53 mg, 521.94 μmol, 2 eq.), and DIC (70.72 μL, 456.70 μmol, 1.75 eq.) were added respectively at room temperature and stirred under N₂. After 18 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and diethyl ether as eluent. The product, 38, was obtained as aa yellow solid with 58% yield.

FT-IR (Neat): v (cm⁻¹)=2956, 2920, 1719, 1698, 1693, 1650, 1644, 1632, 1608, 1537, 1511, 1434, 1409, 1361, 1326, 1278, 1142; ¹H-NMR (400 MHz, CDCl₃): δ ppm 9.16 (s, NH), 8.13 (s, 1H), 8.00 (d, J=8.56 Hz, 2H), 7.86 (s, 1H), 7.67 (d, J=8.06 Hz, 2H), 7.28-7.41 (m, 4H), 3.90 (s, 3H), 3.38 (s, 3H), 2.75 (s, 3H); ¹³C-NMR (100 MHz, CDCl₃): δ 166.14, 166.05, 157.73, 154.15, 144.55, 140.97, 137.33, 136.55, 135.43, 133.42, 130.58, 129.61, 129.14, 126.04, 125.50, 122.98, 119.81, 118.12, 113.11 (2C), 109.17, 52.33, 38.39, 30.28; HRMS-ESI (m/z): calcd. for C₂₄H₂oBrN₃O₅S₃ =604.9748, found=604.9825.

### Example 2s

### Synthesis of Compound 39 (methyl 4-((5-((4-(4-(benzofuran-2-carbonyl)piperazin-1-yl)phenyl)carbamoyl)-2-bromo-N-methylthiophene)-3-sulfonamido)benzoate)

Compound (e) (100.00 mg, 237.27 μmol, 1.5 eq.) was taken in a flask along with DMF as a solvent (approx. 1.5 mL per 1 mmol). (4-(4-aminophenyl)piperazin-1-yl)(benzofuran-2-yl)methanone (49.34 mg, 153.51 μmol, 1 eq.), HOBT (41.49 mg, 307.03 μmol, 2 eq.), and DIC (41.60 μL, 268.65 μmol, 1.75 eq.) were added respectively at room temperature and under N₂. After 18 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and diethyl ether as eluent. The product, 19, was obtained as a yellow solid with 41% yield.

FT-IR (Neat): v (cm⁻¹)=2968, 2930, 2874, 2451, 1650, 1639, 1599, 1572, 1544, 1446, 1409, 1352, 1318, 1306, 1280, 1231, 1215, 1155; ¹H-NMR (400 MHz, CDCl₃): δ ppm 8.98 (s, NH), 7.99-8.03 (m, 2H), 7.83 (s, 1H), 7.66-7.69 (m, 1H), 7.53-7.59 (m, 3H), 7.40-7.45 (m, 1H), 7.32-7.37 (m, 2H), 7.31 (s, 1H), 6.98-7.01 (m, 1H), 6.94 (d, J=9.06 Hz, 2H), 3.91 (s, 3H), 3.79-3.81 (m, 4H), 3.39 (s, 3H), 3.24-3.29 (m, 4H); ¹³C-NMR (100 MHz, DMSO): δ 165.57, 161.10, 158.84, 157.27, 156.76, 153.91, 153.14, 148.15, 144.60, 141.64, 130.01, 128.78, 128.09, 126.66, 126.56, 126.10, 123.72, 123.53, 122.45 (2C), 121.45, 120.36, 116.06 (2C), 111.78 (2C), 111.00, 78.41, 76.66, 64.97, 54.31, 52.24, 23.26; HRMS-ESI (m/z): calcd. for C₃₃H₂₉BrN₄O₇S₂=736.0661, found=736.0753.

### Example 2t

### Synthesis of Compound 40 (methyl 4-((5-((1-benzyl-1H-indo1-5-yl)carbamoyl)-2-bromo-N-methylthiophene)-3-sulfonamido)benzoate)

Compound (e) (65 mg, 149.67 μmol, 1 eq.) was taken in a flask along with DMF as a solvent (approx. 1.5 mL per 1 mmol). 1-Benzyl-1H-indol-5-ylamine (39.92 mg, 179.61 μmol, 1.2 eq.), EDC hydrochloride (57.38mg, 299.34pmo1, 2 eq.), and DMAP (45.71 mg, 374.18 μmol, 2.5 eq.) were added respectively at room temperature and stirred under N₂. After 1.5 hours, the reaction was finished. The solution was quenched with water and then extracted with ethyl acetate. The organic layer was dried with anhydrous MgSO₄ and the product, 40, was purified by column chromatography (eluent Hexane: DCM 1:1) as a white solid with 70% yield.

FT-IR (Neat): v (cm⁻¹)=2919, 2361, 1717, 1542, 1484, 1281, 1185, 795, 703, 696; ¹H-NMR (400 MHz, CDCl₃): δ ppm 8.03 (d, J=8.59 Hz, 2H), 7.90 (br. s., 1H), 7.63 (br. s., 1H), 7.44 (br. s., 1H), 7.35 (br. s., 1H), 7.33 (br. s., 1H), 7.31 (s, 1H), 7.29 (d, J=2.53 Hz, 2H), 7.24 (s, 1H), 7.17 (d, J=3.03 Hz, 1H), 7.08-7.12 (m, 2H), 6.55 (d, J=3.03 Hz, 1H), 5.33 (s, 2H), 3.91 (s, 3H), 3.40 (s, 3H); ¹³C-NMR (100 MHz, CDCl₃): δ 163.57, 160.56, 142.20, 140.87, 139.93, 139.85, 137.02, 132.76, 131.24, 129.55, 129.49, 128.48, 128.36, 127.38, 127.26, 125.96, 124.93, 123.70, 123.20, 122.79, 122.16, 116.83, 115.11, 110.50, 107.73, 102.23, 60.98, 60.36, 30.43; HRMS-ESI (m/z): calcd. for C₂₉H₂₄BrN₃O₅S₂=637.0341, found=637.0281.

### Example 3

### Synthesis of Compounds 1-8 in Table 1

Compounds 1-8 were produced using the following scheme.

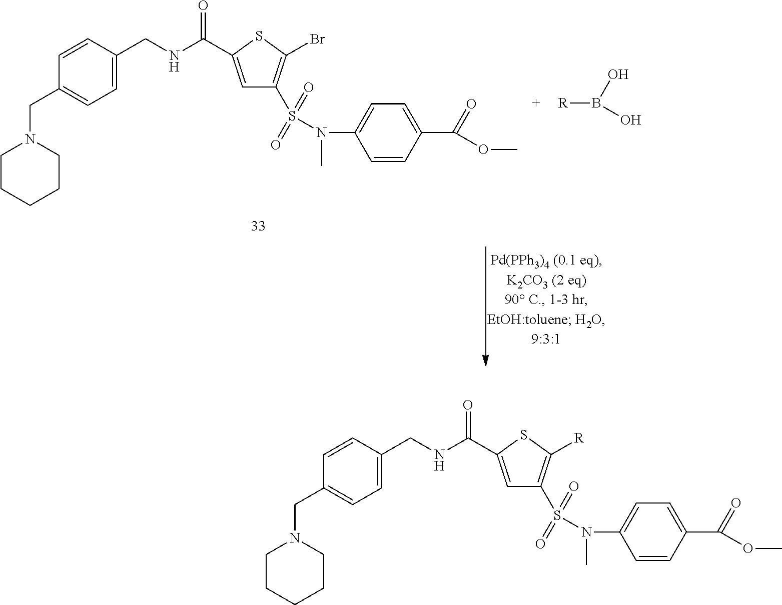

### Example 3a

### Synthesis of Compound 1 (methyl 4-((N-methyl-2-(4-morpholinophenyl)-5-((4-(piperidin-1-ylmethyl)benzyl)carbamoyl)thiophene)-3-sulfonamido)benzoate)

Compound 33 (40 mg, 64.46 μmol, 1 eq.) was taken in a round bottom flask along with a solvent mixture (7 mL) of ethanol, toluene and water (9:3:1). (4-morpholinophenyl) boronic acid (16.01 mg, 77.35 μmol, 1.2 eq.) and K₂CO₃ (26.72 mg, 193.37 μmol, 3 eq.) were added to the solution. Tetrakis (triphenylphosphine) palladium (7.45 mg, 6.45 μmol, and 0.10 eq.) was then added under N₂ and the solution was stirred for 2 hours at 90° C. at which point TLC and LC-MS showed completion of the reaction. The solution was quenched with water and the water layer was extracted with DCM (×3). The combined organic layer was dried with MgSO₄ and the product, 1 was purified by column chromatography (using dichloromethane: methanol, up to 3%, as an eluent) as a yellow solid with 80% yield.

FT-IR (Neat): v (cm⁻¹)=2923, 2850, 1736, 1711, 1698, 1693, 1655, 1643, 1632, 1604, 1561, 1546, 1537, 1508, 1503, 1441, 1346, 1330, 1263, 1225, 1178; ¹H-NMR (400 MHz, CDCl₃): δ ppm 7.78-7.83 (m, 2H), 7.67 (s, 1H), 7.34-7.37 (m, 2H), 7.29-7.32 (m, 4H), 7.00 (d, J=8.81 Hz, 2H), 6.70-6.75 (m, 2H), 6.58 (br. s., NH), 4.58 (d, J=5.54 Hz, 2H), 3.91 (s, 3H), 3.84-3.88 (m, 4H), 3.65 (s, 2H), 3.16-3.21 (m, 4H), 3.01 (s, 3H), 2.55 (br. s., 4H), 1.64-1.71 (m, 4H), 1.47 (br. s., 2H); ¹³C-NMR (100 MHz, CDCl₃): δ 165.80, 160.50, 152.90, 152.07, 149.96, 147.97, 144.96, 142.00, 140.86, 136.50, 131.23, 130.40, 130.08, 129.35, 128.13, 127.94, 126.85, 124.36 (2C), 120.90, 117.18, 115.26, 114.88, 114.08, 68.70, 66.68 (2C), 61.14, 53.79 (2C), 48.05 (2C), 37.60, 29.73, 24.94, 23.70, 23.29; HRMS-ESI (m/z): calcd. for C₃₇H₄₂N₄O₆S₂=702.2546, found=702.2611.

### Example 3b

### Synthesis of Compound 2 (methyl 4-((N-methyl-2-(4-(piperidin-1-yl)phenyl)-5-((4-(piperidin-1-ylmethyl)benzyl)carbamoyl)thiophene)-3-sulfonamido)benzoate)

Compound 33 (40 mg, 64.46 μmol, 1 eq.) was taken in a round bottom flask along with a solvent mixture (3.5 mL) of ethanol, toluene and water (9:3:1). 1-(4-(4,4,5,5-tetramethyl-1,3,2-dioxaborolan-2-yl)phenyl)piperidine (22.21 mg, 77.35 μmol, 1.2 eq.) and K₂CO₃ (26.72 mg, 193.37 μmol, 3 eq.) were added to the solution. Tetrakis (triphenylphosphine) palladium (7.45 mg, 6.45 μmol, and 0.10 eq.) was then added under N₂ and the solution was stirred for 1 hour at 90° C. at which point TLC and LC-MS showed completion of the reaction. The solution was quenched with water and the water layer was extracted with DCM (×3). The combined organic layer was dried with MgSO₄ and the product, 2, was purified by column chromatography (using dichloromethane: diethyl ether 1:1) as a yellow solid with 70% yield.

FT-IR (Neat): v (cm⁻¹)=2925, 2854, 1736, 1720, 1698, 1693, 1655, 1650, 1638, 1631, 1604, 1572, 1561, 1537, 1519, 1509, 1461, 1440, 1346, 1247, 1231, 1178; ¹H-NMR (400 MHz, CDCl₃): δ ppm 7.78-7.84 (m, 2H), 7.61 (s, 1H), 7.32-7.34 (m, 2H), 7.28-7.31 (m, 4H), 6.98-7.01 (m, 2H), 6.72-6.78 (m, 2H), 6.26 (br. s., NH), 4.58 (d, J=5.54 Hz, 2H), 3.91 (s, 3H), 3.50 (s, 2H), 3.20-3.26 (m, 4H), 2.99 (s, 3H), 2.37-2.43 (m, 4H), 1.61-1.73 (m, 8H), 1.55-1.61 (m, 4H); ¹³C-NMR (100 MHz, CDCl₃): δ 166.25, 160.00, 152.64, 146.38, 144.01, 138.36, 137.51, 135.99 (2C), 131.84, 131.15, 130.09 (2C), 129.79, 129.42, 127.91, 127.45, 125.51, 125.23, 124.22, 114.41 (2C), 114.18 (2C), 63.31, 54.38, 54.13, 52.12, 51.52, 49.13, 43.96, 37.52, 32.03, 30.31, 29.69, 25.80, 25.48, 24.27; HRMS-ESI (m/z): calcd. for C₃₆H₄₄N₄₀₅S₂=700.2753, found=700.2815.

### Example 3c

### Synthesis of Compound 3 in Table 1—methyl 4-((N-methyl-2-(6-(4-methylpiperazin-1-yl)pyridin-3-yl)-5-((4-(piperidin-1-ylmethyl)benzyl)carbamoyl)thiophene)-3-sulfonamido)benzoate

Compound 33 (40 mg, 64.46 μmol, 1 eq.) was taken in a round bottom flask along with a solvent mixture (3.5 mL) of ethanol, toluene and water (9:3:1). 1-methyl-4-(5-(4,4,5,5-tetramethyl-1,3,2-dioxaborolan-2-yl)pyridin-2-yl)piperazine (23.45 mg, 77.35 μmol, 1.2 eq.) and K₂CO₃ (26.72 mg, 193.37 μmol, 3 eq.) were added to the solution.

Tetrakis (triphenylphosphine) palladium (7.45 mg, 6.45 μmol, and 0.10 eq.) was then added under N₂ and the solution was stirred for 1.5 hours at 90° C. at which point TLC and LC-MS showed completion of the reaction. The solution was quenched with water and the water layer was extracted with DCM (×3). The combined organic layer was dried with MgSO₄ and the product, 3, was purified by column chromatography (using dichloromethane: methanol, up to 3%, as an eluent) as a yellow solid with 65% yield.

FT-IR (Neat): v (cm⁻¹)=2936, 2845, 2798, 1736, 1719, 1712, 1698, 1693,1678, 1655, 1650, 1632, 1621, 1597, 1572, 1561, 1546, 1537, 1519, 1511, 1438, 1413, 1357, 1317, 1238, 1174 ; ¹H-NMR (400 MHz, METHANOL-d): δ ppm 8.01-8.03 (m, 1H), 7.96-8.00 (m, 1H), 7.78-7.84 (m, 2H), 7.43-7.48 (m, 1H), 7.35-7.39 (m, 4H), 7.15 (d, J=8.56 Hz, 2H), 6.56-6.62 (m, 1H), 4.56 (s, 2H), 3.94 (s, 3H), 3.64 (s, 2H), 3.58-3.63 (m, 4H), 3.15 (s, 3H), 2.57 (d, J=4.53 Hz, 8H), 2.39 (s, 3H), 1.61-1.70 (m, 4H), 1.52 (d, J=4.78 Hz, 2H); ¹³C-NMR (100 MHz, METHANOL-d): δ 167.67, 162.61, 160.26, 151.17, 149.59, 146.51, 140.38, 139.56, 138.57, 133.81, 133.76, 131.42, 131.31, 131.04, 131.01, 129.56, 129.26, 128.76, 126.19 (2C), 117.12 (2C), 106.88, 63.93, 55.65 (2C), 55.10 (2C), 52.77, 46.19, 45.49, 44.28, 37.96, 29.57, 26.12 (2C), 24.80; HRMS-ESI (m/z): calcd. for C₃₇H₄₄N₆O₆S₂=716.2815, found=716.2883.

### Example 3d

### Synthesis of Compound 4 (methyl 4-((N-methyl-2-(4-(morpholine-4-carbonyl)phenyl)-5-((4-(piperidin-1-ylmethyl)benzyl)carbamoyl)thiophene)-3-sulfonamido)benzoate

Compound 33 (40 mg, 64.46 μmol, 1 eq.) was taken in a round bottom flask along with a solvent mixture (3.5 mL) of ethanol, toluene and water (9:3:1). (4-(morpholine-4-carbonyl)phenyl)boronic acid (18.18 mg, 77.35 μmol, 1.2 eq.) and K₂CO₃ (26.72 mg, 193.37 μmol, 3 eq.) were added to the solution. Tetrakis (triphenylphosphine) palladium (7.45 mg, 6.45 μmol, and 0.10 eq.) was then added under N₂ and the solution was stirred for 3 hours at 90° C. at which point TLC and LC-MS showed completion of the reaction. The solution was quenched with water and the water layer was extracted with DCM (×3). The combined organic layer was dried with MgSO₄ and the product, 4, was purified by column chromatography (using dichloromethane: methanol (up to 4%) as an eluent) as a yellow solid with 72% yield.

FT-IR (Neat): v (cm⁻¹)=2930, 2854, 1712, 1698, 1693, 1655, 1639, 1606, 1572, 1561, 1537, 1513, 1503, 1461, 1432, 1359, 1228, 1261, 1177; ¹H-NMR (400 MHz, METHANOL-d): δ ppm 7.73-7.78 (m, 2H), 7.58 (d, J=1.51 Hz, 1H), 7.25-7.29 (m, 2H), 7.23-7.25 (m, 4H), 7.20-7.23 (m, 2H), 6.93-6.98 (m, 2H), 6.58 (br. s., NH), 4.50 (d, J=5.54 Hz, 2H), 3.84 (s, 3H), 3.67 (d, J=12.34 Hz, 4H), 3.56 (d, J=9.06 Hz, 4H), 3.46 (s, 2H), 2.97 (s, 3H), 2.36 (br. s., 4H), 1.50-1.57 (m, 4H), 1.38 (d, J=5.04 Hz, 2H); ¹³C-NMR (100 MHz, METHANOL-d): 0 171.49, 167.26, 162.43, 152.17, 146.97, 146.35, 140.16, 139.03, 137.42, 137.03, 134.32, 133.57, 131.78, 131.31, 131.20, 131.15, 130.49, 129.94, 129.62, 128.70, 127.68 (2C), 126.58, 110.64, 109.12, 67.78, 64.22, 62.38, 55.24 (2C), 52.81, 44.34 (2C), 38.23, 30.69, 26.36, 25.04, 22.21; HRMS-ESI (m/z): calcd. for C₃₈H₄₂N₄O₇S₂=730.2495, found=730.2558.

### Example 3e

### Synthesis of Compound 5 (methyl 4-((N-methyl-2-(2-(4-methylpiperazin-1-yl)pyridin-4-yl)-5-((4-(piperidin-1-ylmethyl)benzyl)carbamoyl)thiophene)-3-sulfonamido)benzoate)

Compound 33 (40 mg, 64.46 μmol, 1 eq.) was taken in a round bottom flask along with a solvent mixture (3.5 mL) of ethanol, toluene and water (9:3:1). (2-(4-methylpiperazin-1-yl)pyridin-4-yl)boronic acid (17.10 mg, 77.35 μmol, 1.2 eq.) and K₂CO₃ (26.72 mg, 193.37 μmol, 3 eq.) were added to the solution. Tetrakis (triphenylphosphine) palladium (7.45 mg, 6.45 μmol, and 0.10 eq.) was then added under N₂ and the solution was stirred for 3 hours at 90° C. at which point TLC and LC-MS showed completion of the reaction. The solution was quenched with water and the water layer was extracted with DCM (×3). The combined organic layer was dried with MgSO₄ and the product, 5 was purified by column chromatography (using ethyl acetate: methanol (up to 3%) as an eluent) as a brown solid with 71% yield.

FT-IR (Neat): v (cm⁻¹)=2923, 2851, 1741, 1736, 1720, 1698, 1693, 1678, 1656, 1650, 1632, 1620, 1598, 1972, 1562, 1537, 1519, 1503, 1492, 1462, 1445, 1390, 1357, 1324, 1274, 1177; ¹H-NMR (400 MHz, METHANOL-d): δ ppm 8.05 (s, 1H), 7.97 (dd, J=5.16, 0.63 Hz, 1H), 7.79-7.83 (m, 2H), 7.33 (s, 4H), 7.11-7.15 (m, 2H), 6.77 (s, 1H), 6.56 (dd, J=5.16, 1.38 Hz, 1H), 4.54 (s, 2H), 3.92 (s, 3H), 3.51 (s, 2H), 3.44-3.49 (m, 4H), 3.14 (s, 3H), 2.51 (t, J=5.04 Hz, 4H), 2.40-2.47 (m, 4H), 2.35 (s, 3H), 1.57-1.63 (m, 4H), 1.44-1.50 (m, 2 H); ¹³C-NMR (100 MHz, Methanol-d): δ 167.10, 160.00, 148.51, 146.45, 140.31, 138.98, 135.22, 131.29 (2C), 131.24, 131.10 (2C), 129.69 (2C), 128.70 (2C), 126.29 (2C), 115.27 (2C), 109.86, 64.27, 62.30, 56.07, 55.61, 55.24 (2C), 46.20, 45.68, 44.33, 38.09, 29.56, 26.39 (2C), 25.09; LCMS-ESI (m/z): calcd. for C₃₇H₄₄N₆O₅S₂=716.28, found=717.3 (M+H⁺)

### Example 3f

### Synthesis of Compound 6 (methyl 4-((N-methyl-2-(2-(piperazin-1-yl)pyridin-4-yl)-5-((4-(piperidin-1-ylmethyl)benzyl)carbamoyl)thiophene)-3-sulfonamido)benzoate)

Compound 33 (40 mg, 64.46 μmol, 1 eq.) was taken in a round bottom flask along with a solvent mixture (3.5 mL) of ethanol, toluene and water (9:3:1). 1-(4-(4,4,5,5-tetramethyl-1,3,2-dioxaborolan-2-yl)pyridin-2-yl)piperazine (22.37 mg, 77.35 μmol, 1.2 eq.) and K₂CO₃ (26.72 mg, 193.37 μmol, 3 eq.) were added to the solution. Tetrakis (triphenylphosphine) palladium (7.45 mg, 6.45 μmol, and 0.10 eq.) was then added under N₂ and the solution was stirred for 2 hours at 90° C. at which point TLC and LC-MS showed completion of the reaction. The solution was quenched with water and the water layer was extracted with DCM (×3). The combined organic layer was dried with MgSO₄ and the product, 6, was purified by column chromatography (using dichloromethane:methanol (up to 5.5%) as an eluent) as a yellow solid with 73% yield.

FT-IR (Neat): v (cm⁻¹)=2930, 2850, 1710, 1636, 1607, 1540, 1448, 1356, 1326, 1275, 1179, 1143, 1103; ¹H-NMR (400 MHz, METHANOL-d): δ ppm 8.06 (s, 1H), 7.98-8.00 (m, 1H), 7.81-7.855 (m, 3H), 7.36 (s, 4H), 7.14-7.18 (m, 2H), 6.76-6.79 (m, 1H), 6.57 (dd, J=5.29, 1.26 Hz, 1H), 4.56 (s, 2H), 3.94 (s, 3H), 3.61 (s, 2H), 3.46-3.51 (m, 4 H), 3.17 (s, 3H), 2.95-3.00 (m, 4H), 2.54 (br. s., 4H), 1.60-1.68 (m, 4H), 1.47-1.54 (m, 2H); ¹³C-NMR (100 MHz, CDCl₃):δ 167.14, 159.97, 150.53, 146.37, 139.52, 135.11, 131.57 (3C), 131.23, 131.09 (3C), 129.69, 128.86 (2C), 126.33 (2C), 109.93, 103.95, 101.70, 75.02, 63.77, 62.34, 55.00 (2C), 45.95, 45.78, 44.30, 38.15, 37.21, 25.99 (2C), 24.69; HRMS-ESI (m/z): calcd. for C₃₆H₄₂N₆O₅S₂=702.2658, found=702.2726.

### Example 3g

### Synthesis of Compound 7 (methyl 4-((N-methyl-2-(4-(4-methylpiperazine-1-carbonyl)phenyl)-5-((4-(piperidin-1-ylmethyl)benzyl)carbamoyl)thiophene)-3-sulfonamido)benzoate)

Compound 33 (40 mg, 64.46 μmol, 1 eq.) was taken in a round bottom flask along with a solvent mixture (3.5 mL) of ethanol, toluene and water (9:3:1). (4-methylpiperazin-1-yl)(4-(4,4,5-trimethyl-1,3,2-dioxaborolan-2-yl)phenyl)methanone (25.54 mg, 77.35 μmol, 1.2 eq.) and K₂CO₃ (26.72 mg, 193.37 μmol, 3 eq.) were added to the solution. Tetrakis (triphenylphosphine) palladium (7.45 mg, 6.45 μmol, and 0.10 eq.) was then added under N₂ and the solution was stirred for 3 hours at 90° C. at which point TLC and LC-MS showed completion of the reaction. The solution was quenched with water and the water layer was extracted with DCM (×3). The combined organic layer was dried with MgSO₄ and the product, 7 was purified by column chromatography (using ethyl acetate: methanol, up to 3%, as an eluent) as a yellow solid with 55% yield.

FT-IR (Neat): v (cm⁻¹)=2922, 2851, 1710, 1635, 1607, 1554, 1489, 1449, 1402, 1383, 1365, 1271, 1177; ¹H-NMR (400 MHz, METHANOL-d): δ ppm 8.02 (d, J=0.50 Hz, 1H), 7.87 (d, J=8.31 Hz, 2H), 7.40 (s, 4H), 7.33 (s, 4H), 7.19 (d, J=8.81 Hz, 2H), 4.58 (s, 2H), 3.94 (s, 3H), 3.74 (s, 2H), 3.38-3.59 (m, 4H), 3.18 (s, 3H), 2.67 (br. s., 4H), 2.54 (br. s., 4H), 2.39 (s, 3H), 1.65-1.73 (m, 4H), 1.52-1.59 (m, 2H); ¹³C-NMR (100 MHz, METHANOL-d): δ 171.53, 167.36, 164.74, 152.34, 149.42, 146.46, 141.33, 140.20, 134.47, 133.65, 131.88, 131.81, 131.32, 131.28, 130.87, 130.66, 130.09, 129.04 (2C), 129.04, 127.71, 126.69, 122.12, 120.96, 118.91, 62.47, 55.00 (2C), 46.77, 46.15, 44.41, 41.93, 38.34, 35.91, 31.21, 29.29, 25.85 (2C), 24.53; HRMS-ESI (m/z): calcd. for C₃₉H₄₅N₅O₆S_{=743.2811}, found=743.2885.

### Example 3h

### Synthesis of Compound 8 (methyl 4-((2-(4-cyanophenyl)-N-methyl-5-((4-(piperidin-1-ylmethyl)benzyl)carbamoyl)thiophene)-3-sulfonamido)benzoate)

Compound 33 (40 mg, 64.46 μmol, 1 eq.) was taken in a round bottom flask along with a solvent mixture (3.5 mL) of ethanol, toluene and water (9:3:1). (4-cyanophenyl)boronic acid (11.37 mg, 77.35 μmol, 1.2 eq.) and K₂CO₃ (26.72 mg, 193.37 μmol, 3 eq.) were added to the solution. Tetrakis (triphenylphosphine) palladium (7.45 mg, 6.45 μmol, and 0.10 eq.)was then added under N₂ and the solution was stirred for 2 hours 30 minutes at 90° C. at which point TLC and LC-MS showed completion of the reaction. The solution was quenched with water and the water layer was extracted with DCM (×3). The combined organic layer was dried with MgSO₄ and the product, 8, was purified by column chromatography (using ethyl acetate: methanol, up to 3%, as an eluent) as a white solid with 67% yield.

FT-IR (Neat): v (cm⁻¹)=3421, 2931, 1755, 1655, 1605, 1543, 1518, 1501, 1451, 1417, 1356, 1326, 1273, 1179, 1144; ¹H-NMR (400 MHz, METHANOL-d): δ ppm 8.05 (s, 1H), 7.81-7.84 (m, 2H), 7.55-7.59 (m, 2H), 7.41-7.44 (m, 2H), 7.39 (s, 4 H), 7.16-7.20 (m, 2H), 4.58 (s, 2H), 3.98 (s, 3H), 3.68 (s, 2H), 3.19 (s, 3H), 2.60 (br. s., 4H), 1.43-1.48 (m, 6H); ¹³C-NMR (24 kHz, MeOD): δ 169.35, 167.21, 150.62, 134.84, 133.62, 132.57, 132.42 (3C), 131.64 (2C), 130.82, 129.88 (2C), 128.90 (2C), 126.55, 119.06, 112.71, 110.70, 69.13, 54.93, 40.20 (2C), 31.65, 30.16, 25.82, 24.97, 24.05; HRMS-ESI (m/z): calcd. for C₃₄H₃₄N₄O₅S₂=642.1971, found=642.2036

### Example 4

Synthesis of Compounds 9-16 in Table 1

In order to avoid the extensive purification and separation stages, compounds 9 -16 in Table 1 were prepared using the following reaction scheme.

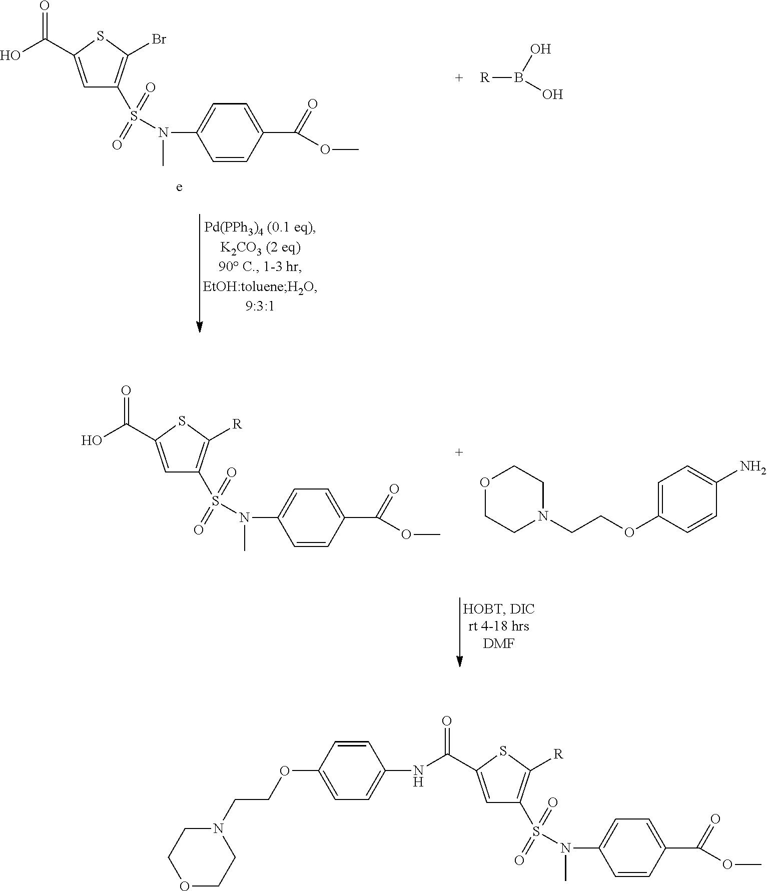

### Example 4a

### Synthesis of Compound 9 (methyl 4-((N-methyl-5-((4-(2-morpholinoethoxy)phenyl)carbamoyl)-2-(4-morpholinophenyl)thiophene)-3-sulfonamido)benzoate)

Step 1—Synthesis of 4-(N-(4-(methoxycarbonyl)phenyl)-N-methylsulfamoyl)-5-(4-morpholinophenyl)thiophene-2-carboxylic acid

Compound (e) prepared as described in Example 1 step 3 (100 mg, 230.27 μmol, 1 eq.) was taken in a round bottom flask along with a solvent mixture (7 mL) of ethanol, toluene and water (9:3:1). 4-Morpholinophenylboronic acid (57.21 mg, 276.32 μmol, 1.2 eq.) and K₂CO₃ (95.47 mg, 690.81 μmol, 3 eq.) were added to the solution. Tetrakis (triphenylphosphine) palladium (26.61 mg, 23.03 μmol, 0.10 eq.) was then added under N₂ and the solution was stirred for 60 minutes at 90° C. at which point TLC and LC-MS showed completion of the reaction. The solution was quenched with water. The pH of the water layer was 11 and 0.025 N HCl was added to the solution to lower the pH to 3, and the water layer was extracted with DCM (×3). The combined organic layer was dried with anhydrous MgSO₄ and the product was purified by column chromatography (using dichloromethane: diethyl ether 90:10 as an eluent) as a yellow solid with 62% yield.

FT-IR (Neat): v (cm⁻¹)=3387, 2850, 1710, 1602, 1575, 1527, 1436, 1421, 1379, 1356, 1306, 1267, 1245, 1205, 1172, 1137, 1111; ¹H-NMR (400 MHz, METHANOL-d): δ ppm 7.73 (s, 1H), 7.70-7.74 (m, 2H), 7.16 (d, J=8.8 hz, 2H), 7.04 (d, J=8.8 hz, 2H), 6.71 (d, J=8.8 hz, 2H), 3.86 (s, 3H), 3.76-3.81 (m, 4H), 3.09-3.14 (m, 4H), 3.03 (s, 3H); ¹³C-NMR (100 MHz, METHANOL-d): δ 167.84, 167.07, 153.64, 153.41, 146.92, 133.42, 132.76, 132.44, 132.28, 131.74, 130.95, 128.50, 125.47, 122.95, 115.79, 115.25, 111.79 (2C), 67.92 (2C), 56.06, 52.70, 49.49, 32.13; HRMS-ESI (m/z): calcd. for C₂₄H₂₄N₂O₇S₂=516.1025, found=516.1091.

Step 2

The product of step 1 (80.00 mg, 154.86 μmol, 1.5 eq.) was taken in a flask along with DMF as a solvent. 4-(2-morpholinoethoxy)aniline (22.96 mg, 103.24 μmol, 1 eq.), HOBT (27.90 mg, 206.49 μmol, 2 eq.), and DIC (27.98 μL, 180.67 μmol, 1.75 eq.) were added respectively at room temperature and under N₂. After 18 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and dichloromethane:methanol (up to 2%) as eluent. The product, 9, was a yellow solid with 78% yield.

FT-IR (Neat): v (cm⁻¹)=3517, 2850, 1720, 1647, 1603, 1545, 1477, 1438, 1411, 1378, 1338, 1302, 1274, 1225, 1182, 1170, 1130; ¹H-NMR (400 MHz, CDCl₃): δ ppm 7.96 (s, 1H), 7.76-7.83 (m, 3H), 7.52 (d, J=9.06 Hz, 2H), 7.33 (d, J=8.81 Hz, 2H), 7.00 (d, J=8.56 Hz, 2H), 6.90 (d, J=8.81 Hz, 2H), 6.74 (d, J=8.81 Hz, 2H), 4.12 (t, J=5.67 Hz, 2H), 3.90 (s, 3H), 3.85-3.89 (m, 4H), 3.73-3.77 (m, 4H), 3.17-3.22 (m, 4H), 3.01 (s, 3H), 2.82 (t, J=5.67 Hz, 2H), 2.57-2.62 (m, 4H); ¹³C-NMR (100 MHz, CDCl₃): δ 166.19, 158.55, 153.72, 153.33, 152.09, 144.95, 137.35, 134.22, 131.96, 131.23, 130.42, 130.11, 129.29, 127.63, 124.35, 123.82, 122.08, 120.78, 114.99, 114.03, 113.11, 113.05, 110.39, 111.20, 66.83 (2C), 66.63 (2C), 65.94, 57.67, 54.05 (2C), 52.22, 47.97 (2C), 37.52; HRMS-ESI (m/z): calcd. for C₃₆H₄₀N₄O₈S₂=720.2288, found=720.2349.

### Example 4b

### Synthesis of Compound 10 (methyl 4-((N-methyl-5-((4-(2-morpholinoethoxy)phenyl)carbamoyl)-2-(4-(piperidin-1-yl)phenyl)thiophene)-3-sulfonamido)benzoate)

Step 1—Synthesis of 4-(N-(4-(methoxycarbonyl)phenyl)-N-methylsulfamoyl)-5-(4-(piperidin-1-yl)phenyl)thiophene-2-carboxylic acid

Compound (e) (50 mg, 115.13 μmol, 1 eq.) was taken in a microwave vial along with a solvent mixture (3 mL) of ethanol, toluene and water (9:3:1). 1-[4-(4,4,5,5-Tetramethyl-1,3,2-dioxaborolan-2-yl)phenyl]piperidine (39.68 mg, 138.16μmol, 1.2 eq.) and K₂CO₃ (47.74 mg, 345.40 μmol, 3 eq.) were added to the solution. Tetrakis (triphenylphosphine) palladium (13.30 mg, 11.51 μmol, .10 eq.) was then added under N₂ and incubated in microwave irradiation at 100° C. for 20 minutes. The solution was quenched with water. The pH of the water layer was 11 and 0.025 N HCl was added to the solution to make the pH 2/3; then the water layer was extracted with DCM (×3). The combined organic layer was dried with anhydrous MgSO₄ and the target product was purified by column chromatography (Et₂O with 1% methanol) as a yellow solid with 60% yield.

FT-IR (Neat): v (cm⁻¹)=2935, 2360, 1714, 1603, 1439, 1279, 1114, 885, 773, 698; ¹H-NMR (400 MHz, METHANOL-d): δ ppm 7.86 (s, 1H), 7.77 (d, J=8.8 Hz, 2H), 7.23 (d, J=8.8 Hz, 2H), 7.07 (d, J=8.8 Hz, 2H), 6.76 (d, J=8.8 hz, 2H), 3.88 (s, 3H), 3.19-3.24 (m, 4H), 3.05 (s, 3H), 1.64-1.72 (m, 6H); ¹³C-NMR (100 MHz, METHANOL-d): δ 163.89, 162.23, 150.66, 143.52, 142.11, 135.50, 134.19, 131.23, 130.67, 128.59, 128.34, 126.00, 119.84, 119.01, 113.60 (2C), 110.96, 110.61, 61.10 (2C), 52.94, 28.99, 25.81, 25.08, 22.41; HRMS-ESI (m/z): calcd. for C₂₅H₂₆N₂O₆S₂=514.1232, found=514.1296.

Step 2

The product from step 1 (80.00 mg, 155.46 μmol, 1.5 eq.) was taken in a flask along with DMF as a solvent. 4-(2-morpholinoethoxy)aniline (23.04 mg, 103.64 μmol, 1 eq.), HOBT (28.01 mg, 207.28 μmol, 2 eq.), and DIC (28.08 μL, 181.37 μmol, 1.75 eq.) were added respectively at room temperature and under N₂. After 15 hours, the reaction was completed. The solution was quenched with water and then extracted with ethyl acetate. The organic layer was combined and dried with anhydrous MgSO₄ and concentrated in vacuo. The crude solid was purified through column chromatography using silica gel and dichloromethane:methanol (up to 2%) as eluent. The product, 10, was obtained as an orange solid with 72% yield.

FT-IR (Neat): v (cm⁻¹)=2915, 2849, 1736, 1719, 1693, 1686, 1682, 1655, 1651, 1632, 1600, 1573, 1537, 1511, 1505, 1466, 1432, 1411, 1329, 1274, 1195; ¹H-NMR (400 MHz, CDCl₃): δ ppm 8.00 (br. s., NH), 7.78-7.84 (m, 3H), 7.53 (d, J=8.81 Hz, 2H), 7.32 (dd, J=8.69, 3.15 Hz, 2H), 7.00 (d, J=8.81 Hz, 2H), 6.87-6.93 (m, 2H), 6.76 (dd, J=8.81, 5.04 Hz, 2H), 4.12 (t, J=5.67 Hz, 2H), 3.89 (s, 3H), 3.72-3.77 (m, 4H), 3.23 (d, J=4.28 Hz, 4H), 3.00 (d, J=1.76 Hz, 3H), 2.81 (t, J=5.67 Hz, 2H), 2.56-2.61 (m, 4H), 1.60 -1.73 (m, 6H); ¹³C-NMR (100 MHz, CDCl₃): δ 166.22, 162.85, 155.94, 152.67, 144.95, 143.07, 142.59, 136.70, 131.77, 131.17 (2C), 130.12 (2C), 127.95, 127.56, 124.57, 124.28 (2C), 120.13, 119.46, 115.03 (2C), 114.40 (2C), 66.91 (2C), 66.07, 61.05, 57.64, 54.09 (2C), 52.13, 49.11, 31.99, 25.47 (2C), 24.27 HRMS-ESI (m/z): calcd. for C₃₇H₄₂N₄O₇S₂=718.2495, found=718.2555.

### Example 4c

### Synthesis of Compound 11 (methyl 4-((N-methyl-2-(6-(4-methylpiperazin-1-yl)pyridin-3-yl)-5-((4-(2-morpholinoethoxy)phenyl)carbamoyl)thiophene)-3-sulfonamido)benzoate)

Step 1—Synthesis 4-(N-(4-(methoxycarbonyl)phenyl)-N-methylsulfamoyl)-5-(6-(4-methylpiperazin-1-yl)pyridin-3-yl)thiophene-2-carboxylic acid

Compound (e) (100 mg, 230.27 μmol, 1 eq.) was taken in a round bottom flask along with a solvent mixture (7 mL) of ethanol, toluene and water (9:3:1). 2-(4-Methylpiperazin-1-yl)pyridine-5-boronic acid Pinnacol ester (83.78 mg, 276.32 μmol, 1.2 eq.) and K₂CO₃ (95.47 mg, 690.81 μmol, 3 eq.) were added to the solution. Tetrakis (triphenylphosphine) palladium (26.61 mg, 23.03 μmol, and 0.10 eq.) was then added under N₂ and the solution was stirred for 4 hours 30 minutes at 90° C. at which point TLC and LC-MS showed completion of the reaction. The pH of the water layer was 11 and 0.025 N HCl was added to the solution to lower the pH to 3, and the water layer was extracted with DCM (×3). The combined organic layer was dried with anhydrous MgSO₄ and the target product, was purified by column chromatography (using dichloromethane: methanol (upto 5%) as an eluent) as a brown solid with 79% yield.

FT-IR (Neat): v (cm⁻¹)=3348, 2929, 2850, 1710, 1597, 1546, 1441, 1352, 1278, 1203, 1171, 1138; ¹H-NMR (400 MHz, METHANOL-d): δ ppm 7.91 (dd, J=6.42, 2.39 Hz, 1H), 7.72-7.78 (m, 3H), 7.42 (dt, J=8.81, 2.77 Hz, 1H), 7.10 (dd, J=8.81, 1.01 Hz, 2H), 6.61 (dd, J=9.06, 4.28 Hz, 1H), 3.89 (s, 3H), 3.63-3.69 (m, 4H), 3.10 (s, 3H), 2.79-2.85 (m, 4H), 2.56 (d, J=2.52 Hz, 3H); ¹³C-NMR (100 MHz, METHANOL-d): δ 167.11, 163.95, 155.44, 149.46, 145.73, 140.60, 137.94, 135.62, 133.71, 131.01, 130.97, 129.39, 126.01, 125.56, 118.29, 115.44, 107.09, 62.32, 55.20, 52.81, 45.38, 44.84, 37.88, 35.88; HRMS-ESI (m/z): calcd. for C₂₄H₂₆N₄O₆S₂=530.1294 found=530.1361

Step 2

The product from step 1 (53.71 mg, 101.22 μmol, 1.5 eq.) was taken in a flask along with DMF as a solvent. 4-(2-morpholinoethoxy)aniline (15.00 mg, 67.48 μmol, 1 eq.), HOBT (18.24 mg, 134.96 μmol, 2 eq.), and DIC (18.29 μL, 118.09 μmol, 1.75 eq.) were added respectively at room temperature and stirred under N₂. After 16 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and dichloromethane:methanol (up to 2%) as an eluent. The product, 11, was obtained as a brown solid with 70% yield.

FT-IR (Neat): v (cm⁻¹)=2939, 2845, 1706, 1597, 1545, 1510, 1435, 1389, 1281, 1254, 1231, 1185, 1134; ¹H-NMR (400 MHz, CDCl₃): δ ppm 8.02-8.06 (m, 1H), 7.95 (s, 1H), 7.81-7.87 (m, 2H), 7.48-7.53 (m, 1H), 7.05-7.09 (m, 2H), 6.73-6.78 (m, 2H), 6.63 (d, J=8.56 Hz, 2H), 6.41-6.45 (m, 1H), 4.04 (t, J=5.79 Hz, 2H), 3.90 (s, 3H), 3.72-3.76 (m, 4H), 3.58-3.63 (m, 4H), 3.11 (s, 3H), 2.77 (t, J=5.67 Hz, 2H), 2.57 (t, J=4.91 Hz, 4H), 2.52 (t, J=4.91 Hz, 4H), 2.37 (s, 3H); ¹³C-NMR(100MHz, CDCl₃): δ 165.82, 160.96, 158.71, 158.66, 148.14, 144.48, 144.41, 138.65 (2C), 134.89, 132.49, 132.38, 130.51, 129.80, 127.95, 127.59, 124.25 (2C), 115.99, 115.45, 115.00, 114.99, 104.79, 66.56 (2C), 66.03, 60.77, 57.46, 54.32, 53.71, 52.31, 51.89, 45.78, 44.14 (2C), 37.20 HRMS-ESI (m/z): calcd. for C₃₆H₄₂N₆O7S₂=734.2556, found=734.2633.

### Example 4d

### Synthesis of Compound 12 (methyl 4-((N-methyl-2-(4-(morpholine-4-carbonyl)phenyl)-5-((4-(2-morpholinoethoxy)phenyl)carbamoyl)thiophene)-3-sulfonamido)benzoate)

Step 1—Synthesis of 4-(N-(4-(methoxycarbonyl)phenyl)-N-methylsulfamoyl)-5-(4-(morpholine-4-carbonyl)phenyl)thiophene-2-carboxylic acid

Compound (e) (100 mg, 230.27 μmol, 1 eq.) was taken in a round bottom flask along with a solvent mixture (7 mL) of ethanol, toluene and water (9:3:1). 4-(morpholine-4-carbonyl)phenyl boronic acid (64.95 mg, 276.32 μmol, 1.2 eq.) and K₂CO₃ (95.47 mg, 690.81 μmol, 3 eq.) were added to the solution. Tetrakis (triphenylphosphine) palladium (26.61 mg, 23.03 μmol, and 0.10 eq.) was then added under N₂ and the solution was stirred for 30 minutes at 90° C., at which point TLC and LC-MS showed completion of the reaction. The solution was quenched with water. The pH of the water layer was 11 and 0.025 N HCl was added to the solution to lower the pH to 3, and the water layer was extracted with DCM (×3). The combined organic layer was dried with anhydrous MgSO₄ and the target product was purified by column chromatography (using dichloromethane: diethyl ether 90:10 as an eluent) as a white solid with 59% yield.

FT-IR (Neat): v (cm⁻¹)=2920, 2857, 1723, 1605, 1578, 1553, 1516, 1467, 1441, 1427, 1362, 1302, 1273, 1252, 1216, 1174, 1139; ¹H-NMR (400 MHz, DMSO-d): δ ppm 7.87 (s, 1H), 7.83 (d, J=9.1 hz, 2H), 7.27-7.33 (m, 4H), 7.16 (d, J=8.8 hz, 2H), 3.85 (s, 3H), 3.55-3.70 (m, 8H), 3.11 (s, 3H); ¹³C-NMR (100 MHz, DMSO-d): δ 168.15, 165.51, 161.63, 145.57, 144.46, 136.46, 135.98, 134.80, 132.34, 130.79, 131.03 (2C), 129.75, 127.57, 126.44, 125.25, 122.23, 117.52, 115.23, 65.99, 64.72, 52.22, 48.56, 40.06, 32.10; HRMS-ESI (m/z): calcd. For C₂₅H₂₄N₂O₈S₂=544.0974 found=544.1038

Step 2

The product from step 1 (55.12 mg, 101.22 μmol, 1.5 eq.) was taken in a flask along with DMF as a solvent. 4-(2-morpholinoethoxy)aniline (15.00 mg, 67.48 μmol, 1 eq.), HOBT (18.24 mg, 134.96 μmol, 2 eq.), and DIC (18.29 μL, 118.09 μmol, 1.75 eq.) were added respectively at room temperature and under N₂. After 18 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and dichloromethane:methanol (upto 3%) as eluent. The product, 12, was obtained as a yellow solid with 87% yield.

FT-IR (Neat): v (cm⁻¹)=2950, 2859, 1736, 1719, 1698, 1693, 1677, 1655, 1650, 1638, 1625, 1618, 1604, 1573, 1561, 1537, 1510, 1504, 1474, 1461, 1433, 1413, 1358, 1331, 1275, 1173; ¹H-NMR (400 MHz, CDCl₃): δ ppm 8.07 (br. s., NH), 7.81-7.86 (m, 2H), 7.77 (s, 1H), 7.51 (d, J=8.81 Hz, 2H), 7.32-7.39 (m, 4H), 7.02-7.07 (m, 2H), 6.88-6.93 (m, 2H), 4.12 (t, J=5.67 Hz, 2H), 3.91 (s, 3H), 3.58-3.85 (m, 12H), 3.04 (s, 3H), 2.82 (t, J=5.67 Hz, 2H), 2.57-2.63 (m, 4H) ¹³C-NMR (100 MHz, CDCl₃): δ 169.23, 166.05, 158.20, 151.07, 144.46, 139.33, 137.51, 136.50, 135.05, 132.89, 131.79, 130.42 (2C), 130.25 (2C), 128.38, 126.8 (2C), 124.86 (2C), 122.12, 114.98 (2C), 112.77, 112.65, 66.82 (4C), 65.92, 57.58, 54.03 (2C), 52.33, 48.54, 47.54, 37.71; HRMS-ESI (m/z): calcd. for C₃₇H₄₀N₄O₉S_{=748.2237}, found=748.2295.

### Example 4e

### Synthesis of Compound 13 (methyl 4-((N-methyl-2-(2-(4-methylpiperazin-1-yl)pyridin-4-yl)-5-((4-(2-morpholinoethoxy)phenyl)carbamoyl)thiophene)-3-sulfonamido)benzoate)

Step 1—Synthesis of 4-(N-(4-(methoxycarbonyl)phenyl)-N-methylsulfamoyl)-5-(2-(4-methylpiperazin-1-yl)pyridin-4-yl)thiophene-2-carboxylic acid

Compound (e) (100 mg, 230.27 μmol, 1 eq.) was taken in a round bottom flask along with a solvent mixture (7 mL) of ethanol, toluene and water (9:3:1). 2-(4-Methylpiperazin-1-yl)pyridine-4-boronic acid pinacol ester (88.76 mg, 276.32 μmol, 1.2 eq.) and K₂CO₃ (95.47 mg, 690.81 μmol, 3 eq.) were added to the solution. Tetrakis (triphenylphosphine) palladium (26.61 mg, 23.03 μmol, and 0.10 eq.) was then added under N₂ and the solution was stirred for 3 hours at 90° C. at which point TLC and LC-MS showed completion of the reaction. The pH of the water layer was 11 and 0.025 N HCl was added to the solution to lower the pH to 3, and the water layer was extracted with DCM (×3). The combined organic layer was dried with anhydrous MgSO₄ and the target product was purified by column chromatography (using dichloromethane: methanol, up to 2%, as an eluent) as a white solid with 80% yield.

FT-IR (Neat): v (cm⁻¹)=3355.61, 1703.83, 1597.74, 1445.83, 1353.14, 1280.03, 1169.25; ¹H-NMR (400 MHz, METHANOL-d): δ ppm 7.92 (d, J=5.29 Hz, 1H), 7.72-7.77 (m, 3H), 7.07-7.12 (m, 2H), 6.73-6.76 (m, 1H), 6.52 (dd, J=5.16, 1.38 Hz, 1H), 3.86 (s, 3H), 3.62 (br. s., 4H), 3.11 (s, 3H), 3.01 (t, J=4.66 Hz, 4H), 2.70 (s, 3H); ¹³C-NMR (100 MHz, METHANOL-d): δ 167.35, 167.25, 159.18, 148.36, 146.64, 146.58, 144.88, 142.48, 134.18, 132.25, 131.03, 129.56, 129.24 (2C), 116.57, 116.30, 110.30, 62.32, 54.65, 52.79, 44.52 (2C), 38.04, 29.79; HRMS-ESI (m/z): calcd. for C₂₄H₂₆N₄O₆S₂=530.1294 found=530.1360.

Step 2

The product from step 1 (53.71 mg, 101.22 μmol, 1.5 eq.) was taken in a flask along with DMF as a solvent. 4-(2-morpholinoethoxy)aniline (15.00 mg, 67.48 μmol, 1 eq.), HOBT (18.24 mg, 134.96 μmol, 2 eq.), and DIC (18.29 μL, 118.09 μmol, 1.75 eq.) were added respectively at room temperature and under N₂. After 6 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and dichloromethane:methanol (up to 3%) as eluent. The product, 13 was obtained as a brown solid with 70% yield.

FT-IR (Neat): v (cm⁻¹)=2923, 2850, 1741, 1736, 1719, 1712, 1698, 1693, 1655, 1650, 1644, 1632, 1600, 1573, 1561, 1543, 1537, 1509, 1461, 1413, 1357, 1330, 1275, 1246, 1172; ¹H-NMR (400 MHz, CDCl₃): δ ppm 8.35 (br. s., NH), 8.10 (dd, J=5.16, 2.39 Hz, 1H), 7.97 (s, 1H), 7.82 (dd, J=8.69, 4.66 Hz, 2H), 7.56 (d, J=8.81 Hz, 2H), 7.01 (d, J=8.56 Hz, 2H), 6.90 (d, J=9.06 Hz, 2H), 6.76 (d, J=8.31 Hz, 1H), 6.54 (d, J=5.29 Hz, 1H), 4.14 (t, J=5.54 Hz, 2H), 3.90 (s, 3H), 3.74-3.79 (m, 4H), 3.67 (br. s., 4H), 3.07 (s, 3H), 2.85 (t, J=5.54 Hz, 2H), 2.78 (br. s., 4H), 2.61-2.66 (m, 4H), 2.56 (s, 3H); ¹³C-NMR (100 MHz, CDCl₃): δ 167.70, 159.35, 157.32, 150.61, 148.62, 141.67, 141.24, 135.29, 132.47, 131.14, 131.10 (2C), 129.80, 126.43 (2C), 123.91 (2C), 116.07, 115.85 (2C), 110.09, 73.52, 67.27 (2C), 66.24, 62.36, 62.26, 58.61, 54.98, 54.92, 44.91, 44.66, 38.13, 30.77; HRMS-ESI (m/z): calcd. for C₃₆H₄₂N₆O7S₂=734.2556, found=734.2633.

### Example 4f

### Synthesis of Compound 14 (methyl 4-((N-methyl-5-((4-(2-morpholinoethoxy)phenyl)carbamoyl)-2-(2-(piperazin-1-yl)pyridin-4-yl)thiophene)-3-sulfonamido)benzoate)

Step 1—Synthesis of 4-(N-(4-(methoxycarbonyl)phenyl)-N-methylsulfamoyl)-5-(2-(piperazin-1-yl)pyridin-4-yl)thiophene-2-carboxylic acid

Compound (e) (100 mg, 230.27 μmol, 1 eq.) was taken in a round bottom flask along with a solvent mixture (7 mL) of ethanol, toluene and water (9:3:1). 1-(4-(4,4,5-trimethyl-1,3,2-dioxaborolan-2-yl)pyridin-2-yl)piperazine (79.91 mg, 276.32 μmol, 1.2 eq.) and K₂CO₃ (95.47 mg, 690.81 μmol, 3 eq.) were added to the solution. Tetrakis (triphenylphosphine) palladium (26.61 mg, 23.03 μmol, and 0.10 eq.) was then added under N₂ and the solution was stirred for 2 hours 30 minutes at 90° C. at which point TLC and LC-MS showed completion of the reaction. The pH of the water layer was 11 and 0.025 N HCl was added to the solution to lower the pH to 3, and the water layer was extracted with DCM (×3). The combined organic layer was dried with anhydrous MgSO₄ and the target product was purified by column chromatography (using dichloromethane: methanol (up to 2%) as an eluent) as a yellow solid with 65% yield.

FT-IR (Neat): v (cm⁻¹)=2955, 2920, 2849, 1712, 1698, 1693, 1658, 1639, 1600, 1555, 1541, 1538, 1519, 1503, 1492, 1453, 1420, 1409, 1334, 1307, 1216, 1173; ¹H-NMR (400 MHz, DMSO-d): δ ppm 7.97 (d, J=5.04 Hz, 1H), 7.81 (d, J=8.56 Hz, 2H), 7.25 (s, 1H), 7.17-7.22 (m, 2H), 6.61 (s, 1H), 6.47 (dd, J=5.04, 1.26 Hz, 1H), 3.85 (s, 3H), 3.34-3.38 (m, 4H), 3.10 (s, 2H), 2.77-2.81(m, 3H); ¹³C-NMR (100 MHz, DMSO): δ 165.49, 164.98, 162.06, 158.43, 148.49, 146.99, 145.90, 144.97 (2C), 140.52, 131.35, 129.63, 127.25, 126.97, 124.53, 113.68, 107.48, 55.80, 54.88, 48.56, 44.95, 44.79, 30.66; HRMS-ESI (m/z): calcd. for C₂₃H₂₄N₄O₆S₂=516.1137, found=516.1202.

Step 2

The product from step 1 (100.00 mg, 193.58 μmol, 1.5 eq.) was taken in a flask along with DMF as a solvent. 4-(2-morpholinoethoxy)aniline (28.69 mg, 129.05 μmol, 1 eq.), HOBT (34.88 mg, 258.10 μmol, 2 eq.), and DIC (34.97 μL, 225.84 μmol, 1.75 eq.) were added respectively at room temperature and stirred under N₂. After 17 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and dichloromethane:methanol (up to 5%) as eluent. The product, 14, was obtained as a yellow solid with 55% yield.

FT-IR (Neat): v (cm−1)=2922, 2850, 1714, 1595, 1541, 1445, 1417, 1356, 1312, 1274, 1175, 1140, 1104; 1H-NMR (400 MHz, METHANOL-d): δ ppm 8.02 (d, J=5.04 Hz, 1H), 7.85 (d, J=8.81 Hz, 2H), 7.63 (d, J=7.05 Hz, 1H), 7.54 (s, 1H), 7.21 (d, J=8.81 Hz, 2H), 6.81-6.85 (m, 1H), 6.64 (d, J=5.29 Hz, 1H), 6.18 (dd, J=6.92, 1.38 Hz, 1H), 5.98 (s, 1H), 3.91 (s, 3H), 3.82-3.87 (m, 4H), 3.50-3.55 (m, 4H), 3.44-3.49 (m, 4H), 3.35 (d, J=0.76 Hz, 2H), 3.15 (s, 3H), 2.98-3.02 (m, 4H), 2.16 (m, 4H); ¹³C-NMR (100 MHz, CDCl₃): δ 167.20, 163.20, 159.71, 149.60, 148.86, 146.44 (2C), 141.36 (2C), 138.94, 134.31, 132.05, 131.31 (2C), 129.94, 129.23, 126.46, 126.32, 116.10 (2C), 110.25, 74.12, 68.71 (2C), 66.47, 61.33 (2C), 57.99, 54.98 (2C), 45.22, 44.81, 31.34; HRMS-ESI (m/z): calcd. for C₃₅H₄₀N₆O₇S₂=720.2400, found=720.2284.

### Example 4 g

### Synthesis of Compound 15 (methyl 4-((N-methyl-2-(4-(4-methylpiperazine-1-carbonyl)phenyl)-5-((4-(2-morpholinoethoxy)phenyl)carbamoyl)thiophene)-3-sulfonamido)benzoate)

Step 1—Synthesis of 4-(N-(4-(methoxycarbonyl)phenyl)-N-methylsulfamoyl)-5-(4-(4-methylpiperazine-1-carbonyl)phenyl)thiophene-2-carboxylic acid

Compound (e) (100 mg, 230.27 μmol, 1 eq.) was taken in a round bottom flask along with a solvent mixture (7 mL) of ethanol, toluene and water (9:3:1). (4-methylpiperazin-1-yl)(4-(4,4,5-trimethyl-1,3,2-dioxaborolan-2-yl)phenyl)methanone (87.38 mg, 276.32 μmol, 1.2 eq.) and K₂CO₃ (95.47 mg, 690.81 μmol, 3 eq.) were added to the solution. Tetrakis (triphenylphosphine) palladium (26.61 mg, 23.03 μmol, and 0.10 eq.) was then added under N₂ and the solution was stirred for 4 hours 15 minutes at 90° C. at which point TLC and LC-MS showed completion of the reaction. The solution was quenched with water. The pH of the water layer was 11 and 0.025 N HCl was added to the solution to lower the pH to 3, and the water layer was extracted with DCM (×3). The combined organic layer was dried with anhydrous MgSO₄ and the target product was purified by column chromatography (using dichloromethane: methanol, up to 5%, as an eluent) as a white solid with 87% yield.

FT-IR (Neat): v (cm⁻¹)=3135.87, 1709.35, 1603.57, 1506.06, 1399.08, 1353.70, 1278.85, 1173.48, 1105.60; ¹H-NMR (400 MHz, METHANOL-d): δ ppm 7.82-7.85 (m, 2H), 7.76 (s, 1 H), 7.33-7.35 (m, 2H), 7.29 (d, J=8.3 hz, 2H), 7.17 (d, J=8.8 hz, 2H), 3.87 (s, 3H), 3.57- 3.84 (br. s., 4H), 3.11 (s, 3H), 3.01-3.09 (m, 4H), 2.71 (s, 3H); ¹³C-NMR (100 MHz, METHANOL-d): δ 171.58, 167.35, 151.24, 146.50, 138.34, 136.15, 135.73, 134.67, 133.56, 132.25, 131.82, 131.14 (2C), 129.76, 127.70, 126.49, 126.45, 118.23, 115.55, 62.37, 54.39, 52.84, 49.87, 48.15, 44.09, 38.15; HRMS-ESI (m/z): calcd. for C₂₆H₂₇N₃O₇S₂=557.1290 found=557.1356.

Step 2

The product from step 1 (56.44 mg, 101.22 μmol, 1.5 eq.) was taken in a flask along with DMF as a solvent. 4-(2-morpholinoethoxy)aniline (15.00 mg, 67.48 μmol, 1 eq.), HOBT (18.24 mg, 134.96 μmol, 2 eq.), and DIC (18.29 μL, 118.09 μmol, 1.75 eq.) were added respectively at room temperature and stirred under N₂. After 15 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and dichloromethane:methanol (up to 6%) as an eluent. The product, 15, was obtained as a white solid with 73% yield.

FT-IR (Neat): v (cm⁻¹)=2936, 2858, 2797, 1719, 1711, 1698, 1655, 1649, 1632, 1600, 1562, 1544, 1537, 1523, 1514, 1503, 1485, 1448, 1358, 1341, 1315, 1299, 1290, 1236, 1227, 1178; ¹H-NMR (400 MHz, METHANOL-d): δ ppm 8.24 (s, 1H), 7.87-7.91 (m, 2H), 7.63 (d, J=8.31 Hz, 2H), 7.35 (d, J=1.01 Hz, 4H), 7.20-7.24 (m, 2H), 7.01 (dd, J=9.06, 1.01 Hz, 2H), 4.21 (t, J=4.0 Hz, 2H), 3.96 (s, 3H), 3.75-3.80 (m, 4H), 3.41-3.67 (m, 4H), 3.24 (s, 3H), 2.84-2.89 (t, J=4.0 Hz, 2H), 2.64-2.69 (m, 4H), 2.56 (br. s., 4H), 2.41 (s, 3H); ¹³C-NMR (100 MHz, METHANOL-d): δ 171.53, 167.38, 160.72, 157.60, 152.56, 146.47, 141.03, 137.75, 134.52, 133.68, 132.50, 131.88, 131.34, 131.30, 130.79, 130.09, 129.76, 127.71, 126.71 (2C), 124.06, 115.97, 114.20, 113.80, 113.01, 67.77 (2C), 66.88, 62.48, 58.91 (2C), 55.30 (3C), 52.92, 52.85, 46.16, 38.39 HRMS-ESI (m/z): calcd. for C₃₈H₄₃N₅O₈S₂=761.2553, found=761.2617.

### Example 4h

### Synthesis of Compound 16 (methyl 4-((2-(4-cyanophenyl)-N-methyl-5-((4-(2-morpholinoethoxy)phenyl)carbamoyl)thiophene)-3-sulfonamido)benzoate)

Step 1—Synthesis of 5-(4-cyanophenyl)-4-(N-(4-(methoxycarbonyl)phenyl)-N-methylsulfamoyl)thiophene-2-carboxylic acid

Compound (e) (100 mg, 230.27 μmol, 1 eq.) was taken in a round bottom flask along with a solvent mixture (7 mL) of ethanol, toluene and water (9:3:1). (4-Cyanophenyl)boronic acid (33.84 mg, 276.32 μmol, 1.2 eq.) and K₂CO₃ (95.47 mg, 690.81 μmol, 3 eq.) were added to the solution. Tetrakis (triphenylphosphine) palladium (26.61 mg, 23.03 μmol, and 0.10 eq.) was then added under N₂ and the solution was stirred for 1 hour at 90° C. at which point TLC and LC-MS showed completion of the reaction. The solution was quenched with water. The pH of the water layer was 11 and 0.025 N HCl was added to the solution to lower the pH to 3, and the water layer was extracted with DCM (×3). The combined organic layer was dried with anhydrous MgSO₄ and the target product was purified by column chromatography (using dichloromethane: methanol (up to 0.6%) as an eluent) as a brown solid with 90% yield.

FT-IR (Neat): v (cm⁻¹)=3097, 2920, 1715, 1605, 1566, 1528, 1507, 1439, 1400, 1359, 1278, 1174, 1141; ¹H-NMR (400 MHz, METHANOL-d): δ ppm 7.73-7.77 (m, 3H), 7.45-7.50 (m, 2H), 7.29-7.34 (m, 2H), 7.09-7.13 (m, 2H), 3.90 (s, 3H), 3.14 (s, 3H); ¹³C-NMR (100 MHz, METHANOL-d): δ 177.22, 174.34, 167.76, 160.10, 151.76, 144.30, 140.07, 133.62, 132.16, 131.45, 131.00, 130.51, 127.82, 125.99, 122.00, 116.03, 114.75, 114.28, 70.28, 53.14, 31.08; HRMS-ESI (m/z): calcd. for C₂₁H₁₆N₂O₆S₂=456.0450 found=456.0518.

Step 2

The product from step 1 (93.00 mg, 203.73 μmol, 1.5 eq.) was taken in a flask along with DMF as a solvent. 4-(2-Morpholinoethoxy)aniline (30.19 mg, 135.82 μmol, 1 eq.), HOBT (36.71 mg, 271.64 μmol, 2 eq.), and DIC (36.81 μL, 237.68 μmol, 1.75 eq.) were added respectively at room temperature and stirred under N₂. After 16 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and dichloromethane:methanol (upto 5%) as eluent. The product, 16, was obtained as a white solid with 82% yield.

FT-IR (Neat): v (cm⁻¹)=3582, 3095, 2809, 2230, 1706, 1654, 1604, 1512, 1453, 1394, 1364, 1279, 1246; ¹H-NMR (400 MHz, DMSO): δ ppm 10.46 (s, 1H), 8.36 (s, 1H), 7.74-7.79 (m, 2H), 7.66-7.71 (m, 2H), 7.61 (d, J=9.06 Hz, 2H), 7.38-7.42 (m, 2H), 7.16-7.21 (m, 2H), 6.95-6.99 (m, 2H), 4.09 (t, J=5.79 Hz, 2H), 3.87 (s, 3H), 3.57-3.60 (m, 4H), 3.14 (d, J=1.51 Hz, 3H), 2.70 (t, J=5.67 Hz, 2H), 2.48 (br. s., 4H) ¹³C-NMR (24kHz, DMSO): δ 165.48, 157.85, 155.18, 151.61, 148.86(2C), 144.17, 140.58, 134.80, 132.74, 131.55, 131.51, 131.12, 130.90, 130.06, 129.71, 129.63, 128.03, 125.31 (2C), 122.04 (2C), 114.61 (2C), 111.97, 66.12 (2C), 65.48, 60.83, 56.99, 53.59 (2C), 34.21; HRMS-ESI (m/z): calcd. for C₃₃H₃2N₄O₇S₂=660.1712, found=661.1789.

### Example 5

### Synthesis of Compounds 17-19 in Table 1

Compounds 17-19 were prepared using the following reaction scheme.

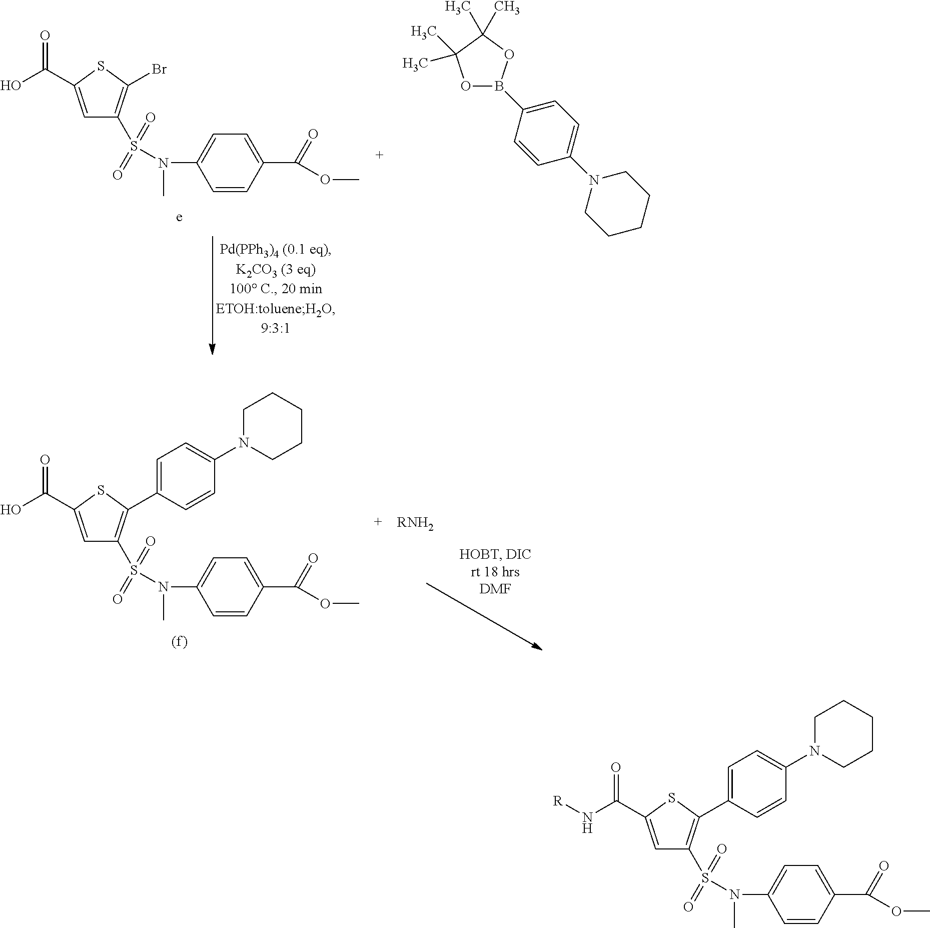

### Example 5a

### Synthesis of Compound 17—(methyl 4-((N-methyl-5-((4-(4-methylpiperazin-1-yl)benzyl)carbamoyl)-2-(4-(piperidin-1-yl)phenyl)thiophene)-3-sulfonamido)benzoate)

Step 1—Synthesis of (f) 4-(N-(4-(methoxycarbonyl)phenyl)-N-methylsulfamoyl)-5-(4-(piperidin-1-yl)phenyl)thiophene-2-carboxylic acid)

Compound (e) (50 mg, 115.13 μmol, 1 eq.) was taken in a microwave vial along with a solvent mixture (3 mL) of ethanol, toluene and water (9:3:1). 1-[4-(4,4,5,5-Tetramethyl-1,3,2-dioxaborolan-2-yl)phenyl]piperidine (39.68 mg, 138.16μmol, 1.2 eq.) and K₂CO₃ (47.74 mg, 345.40 μmol, 3 eq.) were added to the solution. Tetrakis (triphenylphosphine) palladium (13.30 mg, 11.51 μmol, .10 eq.) was then added under N₂ and incubated in microwave irradiation at 100° C. for 20 minutes. The solution was quenched with water. The pH of the water layer was 11, 0.025 N HCl was added to the solution to make the pH 2/3; then the water layer was extracted with DCM (×3). The combined organic layer was dried with anhydrous MgSO₄ and the target product (f) was purified by column chromatography (Et2O with 1% methanol) as a yellow solid with 60% yield.

FT-IR (Neat): v (cm⁻¹)=2935, 2360, 1714, 1603, 1439, 1279, 1114, 885, 773, 698; 41-NMR (400 MHz, METHANOL-d): δ ppm 7.86 (s, 1H), 7.77 (d, J=8.8 Hz, 2H), 7.23 (d, J=8.8 Hz, 2H), 7.07 (d, J=8.8 Hz, 2H), 6.76 (d, J=8.8 hz, 2H), 3.88 (s, 3H), 3.19-3.24 (m, 4H), 3.05 (s, 3H), 1.64-1.72 (m, 6H); ¹³C-NMR (100 MHz, METHANOL-d): δ 163.89, 162.23, 150.66, 143.52, 142.11, 135.50, 134.19, 131.23, 130.67, 128.59, 128.34, 126.00, 119.84, 119.01, 113.60 (2C), 110.96, 110.61, 61.10 (2C), 52.94, 28.99, 25.81, 25.08, 22.41; HRMS-ESI (m/z): calcd. for C₂₅H₂₆N₂O₆S₂=514.1232, found=514.1296.

Step 2

The product (f) from step 1 (190.00 mg, 369.21 μmol, 1.5 eq.) was taken in a flask along with DMF as a solvent. (4-(4-methylpiperazin-1-yl)phenyl)methanamine (50.53 mg, 246.14 μmol, 1 eq.), HOBT (66.52 mg, 492.28 μmol, 2 eq.), and DIC (66.70 μL, 430.75 μmol, 1.75 eq.) were added respectively at room temperature and stirred under N₂. After 5 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and dichloromethane:methanol (upto 3%) as an eluent. The product, 17, was a yellow solid with 80% yield.

FT-IR (Neat): v (cm⁻¹)=2930, 2849, 1736, 1698, 1693, 1655, 1650, 1632, 1603, 1561, 1537, 1503, 1440, 1380, 1349, 1327, 1239, 1177; ¹H-NMR (400 MHz, CDCl₃): δ ppm 7.76-7.83 (m, 2H), 7.58-7.61 (m, 1H), 7.21-7.31 (m, 4H), 6.95-7.01 (m, 2H), 6.91 (d, J=8.56 Hz, 2H), 6.71-6.77 (m, 2H), 6.32 (br. s., NH), 4.50 (d, J=5.54 Hz, 2H), 3.90 (s, 3H), 3.17-3.26 (m, 8H), 2.98 (s, 3H), 2.56-2.62 (m, 4H), 2.34-2.38 (m, 3H), 1.58-1.73 (m, 6H); ¹³C-NMR (100 MHz, CDCl₃): δ 166.25, 160.35, 153.25, 152.58 (2C), 150.88, 145.07, 136.15, 131.63, 131.11, 130.06 (2C), 129.31, 129.15, 128.26, 127.36, 125.91, 124.15, 119.52 (2C), 116.16 (2C), 114.38 (2C), 54.94 (2C), 52.12 (2C), 49.13, 48.86, 46.02, 43.67, 37.47, 31.72, 25.46 (2C), 24.25; HRMS-ESI (m/z): calcd. for C₃₇H₄₃N₅O₅S₂=701.2706, found=701.2772.

### Example 5b

### Synthesis of Compound 18 (methyl 4-((N-methyl-2-(4-(piperidin-1-yl)phenyl)-5-((4-(piperidin-1-yl)phenyl)carbamoyl)thiophene)-3-sulfonamido)benzoate)

The product (f) from Example 5a step 1 (190.00 mg, 369.21 μmol, 1.5 eq.) was taken in a flask along with DMF as a solvent. 4-(piperidin-1-yl)aniline (43.39 mg, 246.14 μmol, 1 eq.), HOBT (66.52 mg, 492.28 μmol, 2 eq.), and DIC (66.70 μL, 430.75 μmol, 1.75 eq.) were added respectively at room temperature and stirred under N₂. After 14 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and diethyl ether as eluent. The product, 18, was obtained as a yellow solid with 60% yield.

FT-IR (Neat): v (cm⁻¹)=2934, 2849, 1716, 1603, 1576, 1527, 1513, 1440, 1399, 1344, 1275, 1236, 1198, 1126, 1103; ¹H-NMR (400 MHz, DMSO-d): δ ppm 10.28 (s, 1H), 8.29 (s, 1H), 7.75-7.77 (d, J=8.8 Hz, 2H), 7.53 (d, J=9.32 Hz, 2H), 7.16 (d, J=9.1 Hz, 4H), 6.92 (d, J=9.06 Hz, 2H), 6.75 (d, J=8.81 Hz, 2H), 3.83 (s, 3H), 3.19 (br. s., 4H), 3.07-3.12 (m, 4H), 3.06 (s, 3H), 1.55-1.65 (m, 12H); ¹³C-NMR (100 MHz, DMSO): δ 165.52, 157.95, 154.47, 152.65, 151.80, 148.45, 144.85, 137.71, 137.68, 131.04, 130.69 (2C), 129.60 (2C), 126.76, 124.36 (2C), 121.40 (2C), 118.73, 115.92 (2C), 113.81 (2C), 54.89 (4C), 49.79, 48.25, 30.67 (4C), 25.24, 24.91; HRMS-ESI (m/z): calcd. for C₃₆H₄₀N₄O₅S₂=672.2440, found=672.2512.

### Example 5c

### Synthesis of Compound 19 (methyl 4-((N-methyl-2-(4-(piperidin-1-yl)phenyl)-5-((3-(piperidin-1-yl)phenyl)carbamoyl)thiophene)-3-sulfonamido)benzoate)

The product (f) from Example 5a step 1 (190.00 mg, 369.21 μmol, 1.5 eq.) was taken in a flask along with DMF as a solvent. 3-(Piperidin-1-yl) aniline (43.39 mg, 246.14 μmol, 1 eq.), HOBT (66.52 mg, 492.28 μmol, 2 eq.), and DIC (66.70 μL, 430.75 μmol, 1.75 eq.) were added respectively at room temperature and stirred under N₂. After 21 hours, the reaction was finished. The solution was quenched with water and then extracted with ethyl acetate. The organic layer was combined and dried with anhydrous MgSO₄ and concentrated in vacuo. The crude solid was purified through column chromatography using silica gel and diethyl ether as eluent. The product, 19, was obtained as a yellow solid with 70% yield.

FT-IR (Neat): v (cm⁻¹)=2922, 2850, 1719, 1657, 1603, 1537, 1494, 1355, 1276, 1197, 1175, 1140; ¹H-NMR (400 MHz, DMSO-d₆); δ ppm 10.25 (s, 1H), 8.33 (s, 1H), 7.72-7.76 (m, 2H), 7.27-7.29 (m, 1H), 7.09-7.17 (m, 6H), 6.74 (d, J=9.06 Hz, 2H), 6.69 (dt, J=7.05, 2.39 Hz, 1H), 3.81 (s, 3H), 3.18 (br. s., 4H), 3.10-3.15 (m, 4H), 3.04 (s, 3H), 1.52-1.62 (m, 12H); ¹³C-NMR (100 MHz, CDCl₃): δ 165.51, 158.42, 153.05, 151.92, 151.83, 144.83, 139.03, 137.49, 137.38, 131.08, 130.71 (2C), 129.60, 129.07, 126.79, 124.39 (2C), 118.66 (2C), 113.80 (2C), 107.64, 101.41, 52.11, 49.50 (2C), 48.23 (2C), 37.11, 34.37, 25.14 (2C), 24.90 (2C), 23.90 (2C).

### Example 6

Fluorescence Polarisation Assay for the Inhibition of STAT3 Dimerisation

The compounds were subjected to an in vitro fluorescent polarisation (FP)-based primary PPI binding screen to assess their ability to inhibit STAT3 dimerisation by interacting with the SH₂ domain. In this FP assay, the unphosphorylated STAT3 monomer (uSTAT3) and a surrogate peptide FAM-pYLPQTV were used to form a ‘pseudo-dimer’. The assay is based on the concept that the compounds were expected to displace the surrogate peptide from the uSTAT3 resulting in a fluorescent read out.

Specifically, for the FP assay, a black (CORNING™) 96 well plate was thoroughly washed with distilled water and allowed to dry. A 100 nM FAM-pYLPQTV working solution was prepared from 10 μM FAM-pYLPQTV stock solution (in DMSO) using PBS pH 7.4 Buffer. 10 μl FAM-pYLPQTV was added to 350 nM uSTAT3 protein, the final concentration of protein in each well, in 90 μl PBS buffer pH 7.4. The MP value of the surrogate dimer complex was measured using a fluorescent plate reader (Envision™, Perkin Elmer™, USA), which provided the base MP value. The inhibitor solution was then added to the well and the assay plate was placed on a shaker for 5 minutes. The MP value for each well was then measured again. A shift in fluorescent values was observed due to displacement of the fluorescently-labelled surrogate peptide by the inhibitors, and the average inhibition for each inhibitor could be calculated as described below:

Base MP value (X) for each well=MP value of Protein and Probe (350 nm STAT3+10 nM FAM-pYLPQTV)−MP value of free FAM-pYLPQTV

Inhibitor MP value (Y) for each well=MP value of Protein+Probe+Inhibitor (350 nm uSTAT3+10 nM FAM-pYLPQTV+2 μL Inhibitor)−MP value of free FAM-pYLPQTV with 2 μL DMSO

% inhibition by any ligand/peptide=(X−Y)/X*100

For comparison purpose, the % inhibition produced by 100 μM pYLKTKF was considered to be 100%, and the inhibition produced by different ligands was reported as relative to 100 μM pYLKTKF.

% Inhibition produced by 100 μM pYLKTKF=A

% Inhibition produced by 100 μM Ligand=B

Relative inhibition (%)=B/A*100

The experiment was carried out in triplicate, and average values were taken as the measure of PPI inhibition.

The results of the FP assay are reported in Table 6.

TABLE 6

| Compound | % Inhibition | Standard deviation | Standard error of mean |
|---|---|---|---|
| RH-06 | 24.24 | 3.14 | 1.81 |
| STA-21 | 32.32 | 2.49 | 1.44 |
| pYLKTKF | 41.63 | 3.23 | 1.86 |
| 20 | 35.64 | 3.99 | 2.30 |
| 1 | 59.36 | 3.88 | 2.24 |
| 2 | 51.25 | 2.45 | 1.41 |
| 3 | 55.19 | 3.61 | 2.08 |
| 4 | 40.61 | 2.66 | 1.53 |
| 5 | 46.86 | 3.07 | 1.77 |
| 6 | 48.94 | 3.20 | 1.85 |
| 7 | 40.61 | 2.66 | 1.53 |
| 8 | 36.44 | 2.38 | 1.37 |
| 9 | 54.15 | 3.54 | 2.04 |
| 10 | 48.94 | 3.20 | 1.85 |
| 11 | 58.31 | 3.82 | 2.20 |
| 12 | 59.36 | 3.88 | 2.24 |
| 13 | 56.23 | 3.68 | 2.12 |
| 14 | 33.32 | 2.18 | 1.26 |
| 15 | 40.61 | 2.66 | 1.53 |
| 16 | 47.90 | 3.13 | 1.81 |
| 17 | 43.73964 | 2.86 | 1.65 |
| 18 | 45.82248 | 3.00 | 1.73 |
| 19 | 43.73964 | 2.86 | 1.65 |
| 40 | 47.07 | 3.49 | 2.02 |
| 21 | 22.69 | 6.94 | 4.01 |
| 22 | 11.73 | 3.79 | 2.18 |
| 23 | 31.30 | 4.3 | 2.48 |
| 24 | 9.42 | 2.6 | 1.50 |
| 25 | 49.62 | 5.84 | 3.37 |
| 26 | 8.24 | 4.2 | 2.42 |
| 27 | 20.27 | 1.95 | 1.12 |
| 28 | 51.26 | 3.89 | 2.24 |
| 29 | 42.90 | 0.94 | 0.54 |
| 30 | 19.46 | 2.60 | 1.50 |
| 31 | 27.27 | 3.79 | 2.19 |
| 32 | 6.18 | 3.90 | 2.25 |
| 33 | 42.34 | 1.37 | 0.79 |
| 34 | 35.40 | 2.32 | 1.33 |
| 35 | 33.32 | 2.18 | 1.26 |
| 36 | 29.15 | 1.91 | 1.10 |
| 37 | 30.20 | 1.97 | 1.14 |
| 38 | 29.22 | 1.37 | 0.79 |
| 39 | 36.44 | 2.38 | 1.37 |

As can be seen from this table, many of the compounds gave greater inhibition than the in-house control compound RH-06, and also the natural hexapeptide pYLKTKF.

### Example 7

MTT Cell viability assay with both MDA-MB-231 breast (STAT3-dependent) and A4 (STAT3-null) cancer cell lines.

After observing significant STAT3 dimerisation inhibitory activity in the preliminary FP assay, the target ligand and related fragments were progressed to an MTT cytotoxicity assay to evaluate in a STAT3-dependent tumour cell line (MDA MB 231, breast cancer) and a STAT3 null A4 cell line. It was anticipated that the compounds would show selective toxicity towards the STAT3-dependent MDA MB 231 cell line and would have lost activity in the STAT3 null A4 cell line if the mechanism of action was STAT3-mediated.

For each cell line, a total of 10,000 cells were seeded for 24 hours before addition of the inhibitors. The cells were grown in normal cell culture conditions at 37° C. under a 5% CO₂ humidified atmosphere using an appropriate medium. The cell count was adjusted to 10⁵ cells/ml, and 5,000-20,000 cells were added per well depending on the cell line. The cells were then incubated for 24 hours, and 1 μl aliquots of the inhibitors were added to the wells in triplicate. After 24 h of continuous exposure to each compound, the cytotoxicity was determined. Absorbance was quantified by spectrophotometry at A=570 nm (Envision™ Plate Reader, PerkinElmer™, USA). IC₅₀ values were calculated by a dose-response analysis using the Prism GraphPad Prism® software package. A SH2 domain interacting small-molecule STA-21 was used as a positive control in place of the natural hexapeptide pYLKTKF, as the peptide did not show any cytotoxicity in the MTT assay at the highest concentration (100 μM) evaluated. The results of the cytotoxicity screen are provided in Table 7

TABLE 7
IC₅₀ values (μM) determined after 48 hours exposure
to compounds in the STAT3-dependent human cancer cell
line MDA-MB-231 and the STAT3-null cell lines.

| | IC₅₀ (MDA-MB-231) | IC₅₀ (A4) | |
|---|---|---|---|
| Compound | μM | μM | Selectivity ratio |
| STA-21 | 18.7 | 65.45 | 3.5 |
| 20 | 10.45 | 58.8 | 5.62 |
| 1 | 2.68 | 75.4 | 28.13 |
| 2 | 2.12 | 84.3 | 39.76 |
| 3 | 1.25 | 65.1 | 52.08 |
| 4 | 1.45 | 77.4 | 53.37 |
| 5 | 2.43 | 57.4 | 23.62 |
| 6 | 0.9 | 73.2 | 81.33 |
| 7 | 6.5 | 65.1 | 10.01 |
| 8 | 12.4 | 34.3 | 2.76 |
| 9 | 2.88 | 89.90 | 31.21 |
| 10 | 2.59 | 67.50 | 26.06 |
| 11 | 7.51 | 66.40 | 8.84 |
| 12 | 3.91 | 75.40 | 19.28 |
| 13 | 1.86 | 74.70 | 40.01 |
| 14 | 14.5 | 78.40 | 5.40 |
| 15 | 10.05 | 84.50 | 8.45 |
| 16 | 1.10 | 35.60 | 32.36 |
| 17 | 4.7 | 64.3 | 13.68 |
| 18 | 3.5 | 45.3 | 12.94 |
| 19 | 3.2 | 34.6 | 10.81 |
| 21 | 23.5 | 64.7 | 2.75 |
| 22 | 34.4 | 73.5 | 2.13 |
| 23 | 12.5 | 54.3 | 4.34 |
| 24 | 34.5 | 33.2 | 0.96 |
| 25 | 3.60 | 43.6 | 12.11 |
| 26 | 14.50 | 25.8 | 1.77 |
| 27 | 8.70 | 58.3 | 6.70 |
| 28 | 6.50 | 42.7 | 6.56 |
| 29 | 5.80 | 53.6 | 9.24 |
| 30 | 24.60 | 82.3 | 3.34 |
| 31 | 32.50 | 24.7 | 0.76 |
| 32 | 7.65 | 74.5 | 9.73 |
| 33 | 4.45 | 94.2 | 21.16 |
| 34 | 7.32 | 65.4 | 8.93 |
| 35 | 4.45 | 38.7 | 8.69 |
| 36 | 7.80 | 45.3 | 5.80 |
| 37 | 5.64 | 55.6 | 9.85 |
| 38 | 11.57 | 54.1 | 4.67 |
| 39 | 4.80 | 43.2 | 9 |

All the compounds evaluated showed a greater cytotoxicity toward the STAT3-dependent MDA MB 231 cell line compared to the STAT3-null A4 cell line, with selectivity ratios between 2.7 to 81.3. Most of the compounds were notably more active than STA-21 which had an IC₅₀ value of 18.7 μM. Compounds 3, 4 and 6 had IC₅₀ values of less than 2 μM with selectivity ratios from 52.08 to 81.33 which were significant.

Furthermore, cytotoxicity values of most of the compounds correlated well with the % inhibition observed in the FP assay.

### Example 8

Effect of Compounds of the Invention on Expression of STAT3 and STAT3-Dependent Genes.

To probe the STAT3-specific cytotoxicity of the mature ligands, the mRNA expression profile of STAT3 and some STAT3 target genes (i.e., bcl-2, cyclin D1, and fascin) were compared to the reference gene GAPDH using a RT-PCR assay. Initially, the MDA-MB-231 cells were stimulated with 500 μg/mL LPS for 24 hours at 37° C. Following this, the compounds were added at 25 μM to the cells and incubated for another 8 hours at 37° C. Finally, PCR was used to determine changes in the gene expression profiles between stimulated-untreated and stimulated-treated cells.

Compounds 3, 4, 6 and 10 were selected for RT-PCR analysis, as these compounds had significant activities in the cell-free FP assay (i.e., greater than 50% inhibition), reasonable cytotoxicity in the STAT3-dependent MDA MB 231 cell line (i.e., IC₅₀ between 0.9 and 2.59 μM), and very good selectivity ratios between the STAT3-dependent MDA MB 231 and STAT3-null A4 cell lines.

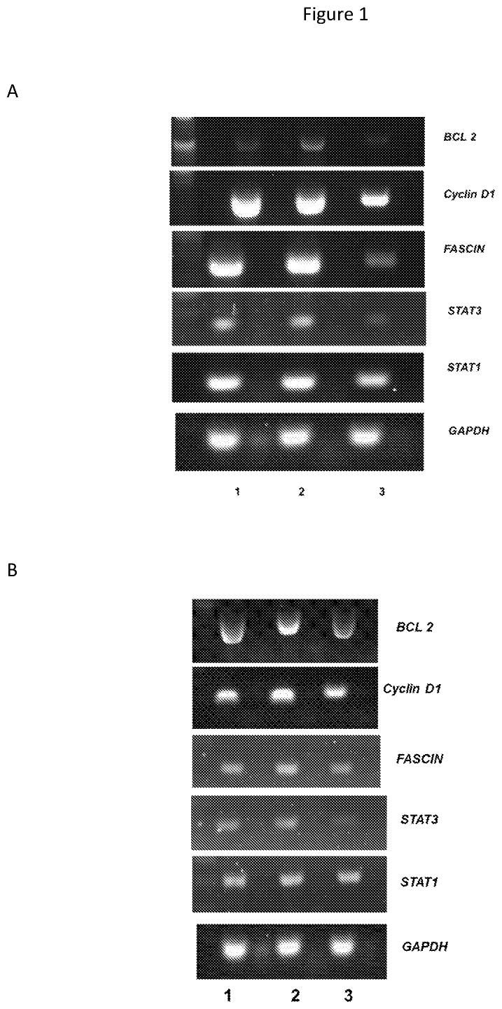

The results are shown in FIG. 1. Compound 3 produced notable downregulation of STAT3 and STAT3-dependent genes as shown in FIG. 1A. Observations showed that the compound down-regulated BCL-2, Cyclin D1 and the expression of Fascin without showing any effect on the house-keeping gene GAPDH. The down-regulation of all three STAT3-dependent genes in treated cells was significant compared to the untreated cells. This suggests a relationship between the inhibition of STAT3-dimerisation inhibition and downregulation of STAT3-dependent genes due to a reduced availability of dimeric STAT3 to act as a transcription factor in the nucleus.

Similar to compound 3, compound 4 also produced notable downregulation of STAT3 and STAT3 dependent genes (FIG. 1B) without showing any effect on the housekeeping gene GAPDH. The effect was more pronounced for BcI-2 and STAT3. Interestingly, the downregulation observed for cyclin D1 and fascin was relatively low compared to 3. The down-regulation appeared to be STAT3 specific as 4 did not downregulate expression of STAT1.

Compound 6 produced the most marked down-regulation of STAT3 and STAT3-dependent genes without showing any effect on both STAT1 and the house-keeping gene GAPDH (FIG. 1C). Almost no expression of STAT3, fascin and cyclin D1, and a very weak expression of BcI-2 were observed after treating the MDA MB 231 cells with 6. The downregulation of STAT3-dependent genes observed for 6 was consistent with its excellent STAT3-diemrisation inhibition and sub-micromolar IC₅₀ in the STAT3-dependent cell line with a high selectivity ratio. Molecular dynamics simulations also revealed strong interactions with the key residues of the STAT3-SH₂ domain. This correlation between biophysical and biological results was very encouraging, and it was decided to progress this molecule to the in vivo xenograft study.

The STAT3-dependent gene down-regulation pattern by compound 10 (FIG. 1D) was comparable to that observed for compound 4. The compound notably down-regulated all three STAT3-dependent genes with a very strong down-regulation observed for BcI-2. The downregulation observed for cyclin al and fascin was marked compared to the untreated cell. It significantly downregulated STAT3 in the treated cell but did not show any effect on the down-regulation of STAT1 and the house-keeping gene GAPDH at the concentration tested.

Overall, the results of the RT-PCR experiments indicate that the compounds of the invention may show selective downregulation of STAT3 and STAT3-dependent genes without any effect on the house-keeping gene GAPDH.

### Example 9

In Vivo Evaluation of Compound 6 in a Human Tumour Xenograft Study

Compound 6 showed remarkable consistency in in silico, cell-free STAT3-dimetrisation inhibition, and selective cytotoxicity against the STAT3-dependent cell line and finally in the RT-PCR assay demonstrating strong down-regulation of STAT3-dependent genes. Therefore, it was decided to carry out a preliminary in vivo efficacy study in mice to evaluate the ability of the molecule to reduce the tumour volume in MDA MB 231 tumour bearing mice. The in vivo tumour xenograft study was carried out in SCID Hairless Outbred (SHO) mice, using an intravenous (IV) dosing regimen.

MDA-MB231 cells were mixed with matrigel (Geltrex, Gibco) before implantation into mice. For tumour generation, 5 million cells in a 50% matrigel mixture were inoculated subcutaneously on both flanks of 8 weeks old SCID Hairless Outbred (SHO) mice purchased from Charles River (Germany). After 2 weeks, the formed tumours had an average diameter of 5-6 mm, and the mice were injected with a 200 μL solution of Compound 6 via the tail vein at a dose level of 0.5 mg/Kg at day one. The injections were repeated on day 3 and day 5. The sizes of the tumours were regularly measured using a caliper every 2 days. The tumour size of the treated mice was compared with that of the control group (7 untreated mice).

The compound produced a significant reduction in tumour volume compared to the control group after three doses. The treated mice did not show any signs of toxicity, and no organ changes were observed after sacrificing the mice. The treated mice remained alive up to 21 days, and did not show any signs further at which point they were culled to comply with the Home Office license.

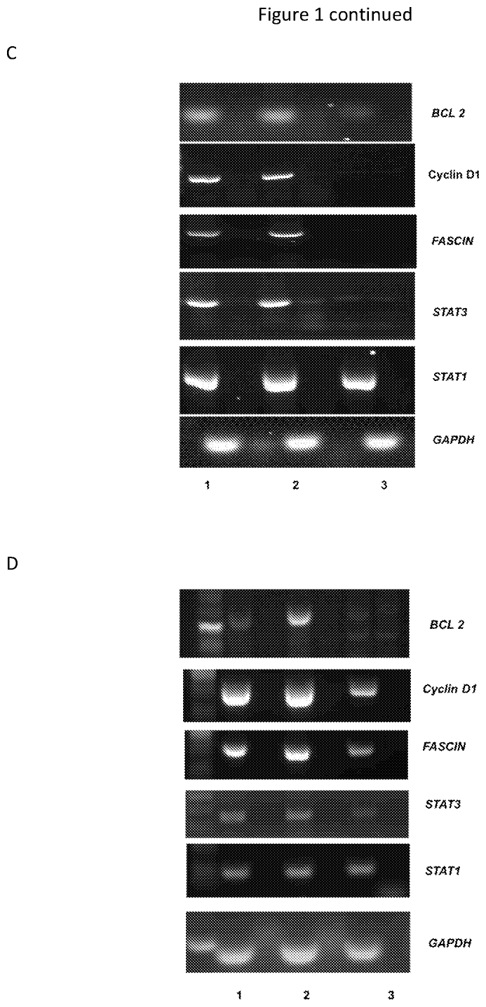

The weight of the treated mice was monitored regularly and the result is shown in FIG. 2. The days of dosing are indicated by vertical dashed lines. The mice initially lost some weight post-treatment, but regained the weight after 14 days.

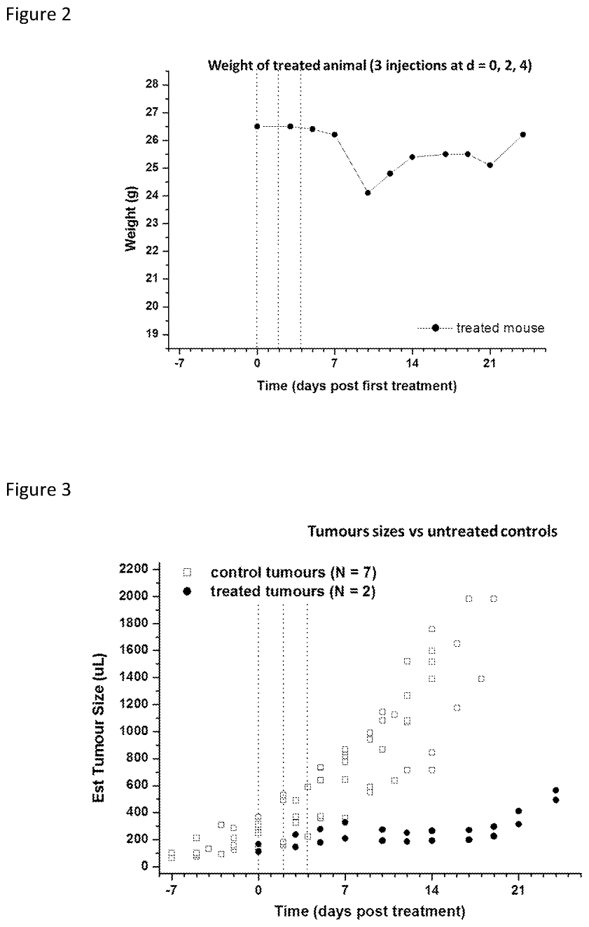

The effect of Compound 6 on tumour size can be viewed in FIG. 3. The volume of the treated tumours remained low and near the baseline throughout the treatment period, while the volume of the untreated tumours increased sharply to 2000 pl after 23 days. Interestingly, there was no significant increase in tumour volume after the dosing was stopped on day 5.

1. A compound of formula (I) 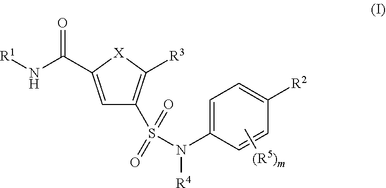
where X is oxygen, sulfur, NR¹¹ or CH₂, where R¹¹ is H or alkyl;
R¹ is an aryl, aralkyl group, heteroaryl group or heteroarylalkyl group; all of which are substituted by one or more groups selected from alkoxycarbonyl, aryl, aralkyl, arylalkoxy, heterocyclyl, heterocyloalkyl or heterocycloalkoxy group, any of which substituent groups may be optionally substituted;
R² is a group of formula COR⁶ where R⁶ is hydrogen or a group OR⁷ where R⁷ is hydrogen, alkyl, alkenyl, alkynyl, cycloalkyl, aryl, heterocyclyl, amino, alkylamino or dialkylamino;
R³ is hydrogen, halo, nitro, cyano, carboxy, optionally substituted alkyl, optionally substituted alkenyl, optionally substituted alkynyl, optionally substituted aryl, optionally substituted cycloalkyl, or optionally substituted heterocyclic group;
R⁴ is hydrogen, C₁₋₄ alkyl or CF₃ group;
R⁵ is a substituent independently selected from hydroxy, C₁₋₄alkyl, C₁₋₄alkoxy, halo, amino, C₁₋₄alkylamino, C₁₋₄dialkylamino, nitro, cyano, thiol, trifluoromethyl
m is 0, 1, 2 or 3;
or a tautomer or a pharmaceutically acceptable salt thereof.

2. A compound according to claim 1 wherein X is sulfur.

3. A compound according to claim 1 or claim 2 wherein R¹ is an aryl, aralkyl group, heteroaryl group or heteroarylalkyl group, which is substituted by an aryl, aralkyl, arylalkoxy, heterocyclyl, heterocyloalkyl or heterocycloalkoxy group, any of which may be optionally substituted by one or more one or more alkyl groups.

4. A compound according to claim 3 wherein R¹ is a group of sub-formula (i) 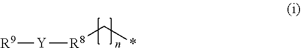
where * is the point of attachment,
n is 0 or an integer of from 1 to 6,
R⁸ is an aryl or heteroaryl group,
Y is a bond, a carbonyl group or an alkylene spacer group of from 1 to 6 atoms, optionally interposed with a heteroatom such as oxygen, nitrogen or sulfur or a carbonyl group; and
R⁹ is an aryl or heterocyclic group, either of which may be optionally substituted by an alkyl group.

5. A compound according to claim 4 wherein n is 0 or 1.

6. A compound according to claim 4 or claim 5 wherein R⁸ is a phenyl group.

7. A compound according to any one of claims 4 to 6 wherein Y is a bond a C₁₋₄alkylene group or a C₁₋₄alkyloxy group.

8. A compound according to any one of claims 4 to 7 wherein R⁹ is a non-aromatic heterocyclic group.

9. A compound according to any one of the preceding claims wherein R² is a group COOR⁷ where R⁷ is a C₁₋₃ alkyl group.

10. A compound according to any one of the preceding claims wherein m is 0 or 1.

11. A compound according to any one of the preceding claims wherein R³ is a group of sub-formula (ii) 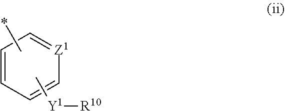
where * is the point of attachment, Z¹ is —CH═ or —N═, Y¹ is a bond, a carbonyl group or an alkylene chain of from 1 to 4 carbon atoms, optionally interposed with a heteroatom such as oxygen, nitrogen or sulfur or a carbonyl group, and R¹⁰ is an optionally susbstituted heterocyclic group.

12. A compound according to claim 11 wherein R¹⁰ is morpholino, piperidinyl, piperazinyl or N-alkylpiperazinyl.

13. A compound according to any one of the preceding claims wherein R⁴ is hydrogen or methyl.

14. A compound according to claim 1 which is selected from Compounds 1-40 in Table 1.

15. A method for preparing a compound according to any one of the preceding claims which method comprises either (a) reacting a compound of formula (II) 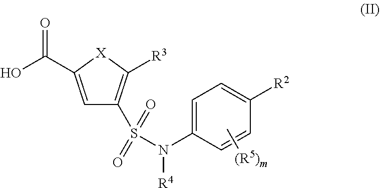
where X, R², R³, R⁴, R⁵ and m are as defined in claim 1, with a compound of formula (III)
R¹—NH₂ (III)
where R¹ is as defined in claim 1, and optionally converting a group R³ to a different such group; or
(b) reacting a compound of formula (XI) 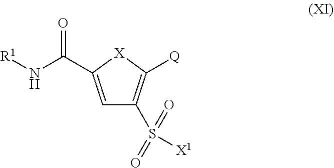
where R¹, R³ and X are as defined in claim 1 and X¹ is a leaving group with a compound of formula (VII) 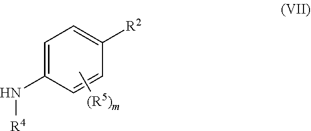
where R², R⁴, R⁵ and m are as defined in claim 1, and optionally converting a group R³ to a different such group; or (c) to prepare compounds of formula (I) where R³ is other than hydrogen, halo or nitro, reacting a compound of formula (X) 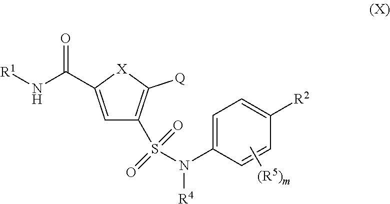
where X, R¹, R², R⁴, R⁵ and m are as defined in claim 1 and Q is a leaving group, in particular a Suzuki leaving group such as halo (in particular bromo) or triflate; with a with a compound of formula (V)
R³—B(OH)₂ (V)
where R³ is as defined above but is other than hydrogen, halo or nitro:
and thereafter, recovering a compound of formula (I) or a pharmaceutically acceptable salt thereof.

16. A method according to claim 15 wherein the compound of formula (II) is prepared by reacting a compound of formula (IV) 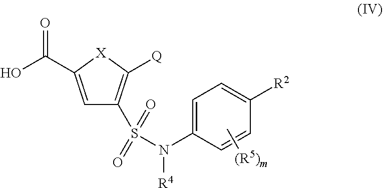
where R², R⁴, R⁵ and m are as defined above, and Q is a leaving group, in particular a Suzuki leaving group such as halo (in particular bromo) or triflate; with a boronic acid of formula (V)
R³—B(OH)₂ (V)
where R³ is as defined in claim 1 but is other than hydrogen, halo or nitro.

17. A compound of formula (II) or (X) as defined in claim 15, or a compound of formula (IV) as defined in claim 16.

18. A compound of formula (I) as defined in claim 1, (IV) as defined in claim 16 or (X) as defined in claim 15 for use in therapy.

19. A pharmaceutical composition comprising a compound of formula (I) as defined in claim 1, (IV) as defined in claim 16 or (X) as defined in claim 15 in combination with a pharmaceutically acceptable carrier.

20. A pharmaceutical composition according to claim 18 which comprises a compound of formula (I).

21. A method of treating a disease or condition by inhibiting SAT3, said method comprising administering to a patient in need thereof, an effective amount of a compound of formula (I) as defined in any one of claims 1 to 13 or a compound according to claim 18, or a pharmaceutical composition according to claim 19 or claim 20.

22. A method according to claim 21 wherein the disease is proliferative disease.

23. A method according to claim 22 wherein the proliferative disease is cancer.
